SEQUENCE LISTING (71 sequences)

SEQ ID NO: 1 - DNA, 1051 bp, Arabidopsis thaliana
aaatcgaaaggcacagcccaacttttcgcaagtcgctgtaaagtttgatttgcttctttttatatacacacatacttctcctccatacactttcctcttcaatcctcagttttttttctaagccctaataccatctcaaagaagagatcaagatttgaaatcaagaagacaccattactcagatcaacatgcttgccgttcaccgtccttcttccgccgtatcagacggagattccgttcagattccgatgatgatcgcgtcgtttcaaaaacgttttccttctctctcacgcgactccacggccgctcgttttcacacacacgaggttggtcctaatcagtgttgctccgccgttattcaagagatctccgctccaatctccaccgtttggtccgtcgtacgccgctttgataacccacaagcttacaaacactttctcaaaagctgtagcgtcatcggcggagacggcgataacgttggtagcctccgtcaagtccacgtcgtctctggtctccccgccgctagctccaccgagagactcgatatcctcgacgacgaacgccacgtcatcagcttcagcgttgttggtggtgaccaccggctctctaactaccgatccgtaacgacccttcacccttctccgatctccgggaccgtcgttgtcgagtcttacgtcgttgatgttcctccaggcaacacaaaggaagagacttgtgacttcgttgacgttatcgtacgatgcaatcttcaatctcttgcgaaaatagccgagaatactgcggctgagagcaagaagaagatgtctctgtgatgagtctttgtcgttgtcgggtagtttcgttagatccgacgtcgttttctagatttttagccgtcgtgtgatctatgttttttcggcttatgtgtgaaaaaaaagttacattagtgaattaatctctcatgcatatcataatccttcttttaatttttgtattttacatatcccataaagaaccgatttggatagccctattccggctttcaccacccaaagataataatattcaaact

SEQ ID NO: 2 - DNA, 624 bp, Arabidopsis thaliana
atgcttgccgttcaccgtccttcttccgccgtatcagacggagattccgttcagattccgatgatgatcgcgtcgtttcaaaaacgttttccttctctctcacgcgactccacggccgctcgttttcacacacacgaggttggtcctaatcagtgttgctccgccgttattcaagagatctccgctccaatctccaccgtttggtccgtcgtacgccgctttgataacccacaagcttacaaacactttctcaaaagctgtagcgtcatcggcggagacggcgataacgttggtagcctccgtcaagtccacgtcgtctctggtctccccgccgctagctccaccgagagactcgatatcctcgacgacgaacgccacgtcatcagcttcagcgttgttggtggtgaccaccggctctctaactaccgatccgtaacgacccttcacccttctccgatctccgggaccgtcgttgtcgagtcttacgtcgttgatgttcctccaggcaacacaaaggaagagacttgtgacttcgttgacgttatcgtacgatgcaatcttcaatctcttgcgaaaatagccgagaatactgcggctgagagcaagaagaagatgtctctgtga

SEQ ID NO: 3 - protein, 207 aa, Arabidopsis thaliana
MLAVHRPSSAVSDGDSVQIPMMIASFQKRFPSLSRDSTAARFHTHEVGPNQCCSAVIQEISAPISTVWSVVRRFDNPQAYKHFLKSCSVIGGDGDNVGSLRQVHVVSGLPAASSTERLDILDDERHVISFSVVGGDHRLSNYRSVTTLHPSPISGTVVVESYVVDVPPGNTKEETCDFVDVIVRCNLQSLAKIAENTAAESKKKMSL

SEQ ID NO: 4 - DNA, 1106 bp, Arabidopsis thaliana
aaatcgaaaggcacagcccaacttttcgcaagtcgctgtaaagtttgatttgcttctttttatatacacacatacttctcctccatacactttcctcttcaatcctcagttttttttctaagccctaataccatctcaaagaagagatcaagatttgaaatcaagaagacaccattactcagatcaacatgcttgccgttcaccgtccttcttccgccgtatcagacggagattccgttcagattccgatgatgatcgcgtcgtttcaaaaacgttttccttctctctcacgcgactccacggccgctcgttttcacacacacgaggttggtcctaatcagtgttgctccgccgttattcaagagatctccgctccaatctccaccgtttggtccgtcgtacgccgctttgataacccacaagcttacaaacactttctcaaaagctgtagcgtcatcggcggagacggcgataacgttggtagcctccgtcaagtccacgtcgtctctggtctccccgccgctagctccaccgagagactcgatatcctcgacgacgaacgccacgtcatcagcttcagcgttgttggtggtgaccaccggctctctaactaccgatccgtaacgacccttcacccttctccgatctccgggaccgtcgttgtcgagtcttacgtcgttgatgttcctccaggcaacacaaaggaagagacttgtgacttcgttgacgttatcgtacgatgcaatcttcaatctcttgcgaaaatagccgagaatactgcggctgagagcaagaagaagatgtctctgtgatgagtctttgtcgttgtcgggtagtttcgttagatccgacgtcgttttctagatttttagccgtcgtgtgatctatgttttttcggcttatgtgtgaaaaaaaagttacattagtgaattaatctctcatgcatatcataatccttcttttaatttttgtattttacatatcccataaagaaccgatttggatagccctattccggctttcaccacccaaagataataatattcaaactgaaagaatgtggttgtgttgtccgctaattaaaagtgtgattttcaagtttaatt

SEQ ID NO: 5 - DNA, 768 bp, Vitis vinifera
ccacttagccctattcctcaaaccccacaagcccctccacctcctcttcctcccatcaatcttaataccaaaagtgaaataaaaatgccctcaaaccctccaaaatcctcgcttgtggttcatagaatcaacagtcctaatagcattaccactgccaccaccgcttctgccgccgcgaataaccataatacttcaaccatgcctcctcacaagcaagttcagaagcggtctcctctaacgagcgccacccaggtccccgacgctgtgtcacgccaccacacccacgttgtcggccccaatcaatgctgctccgccgtcgtccagcaaatcgccgcccctgtctccaccgtctggtccgtcgtccgccgcttcgacaatccacaggcctacaagcacttcgtcaagagttgccacgtcgttgttggcgatggagatgtcggcactctccgcgaggtccacgtcatctccggcctccccgcggccaacagcaccgagcgcctcgaaatcctcgacgacgagcgccatgtcctcagcttcagcgtgatcggtggtgaccaccgcctctccaattaccgatcggtcaccactctccacccctccccctccagcaccggcaccgtggtcctggaatcctatgtcgttgacatacccccaggaaacaccaaagaagacacgtgtgtttttgtcgacaccatcgttcggtgcaacctgcaatcccttgctcagatcgcggagaacgccgccggctgcaagaggtcatcgtcatga

SEQ ID NO: 6 - DNA, 684 bp, Vitis vinifera
atgccctcaaaccctccaaaatcctcgcttgtggttcatagaatcaacagtcctaatagcattaccactgccaccaccgcttctgccgccgcgaataaccataatacttcaaccatgcctcctcacaagcaagttcagaagcggtctcctctaacgagcgccacccaggtccccgacgctgtgtcacgccaccacacccacgttgtcggccccaatcaatgctgctccgccgtcgtccagcaaatcgccgcccctgtctccaccgtctggtccgtcgtccgccgcttcgacaatccacaggcctacaagcacttcgtcaagagttgccacgtcgttgttggcgatggagatgtcggcactctccgcgaggtccacgtcatctccggcctccccgcggccaacagcaccgagcgcctcgaaatcctcgacgacgagcgccatgtcctcagcttcagcgtgatcggtggtgaccaccgcctctccaattaccgatcggtcaccactctccacccctccccctccagcaccggcaccgtggtcctggaatcctatgtcgttgacatacccccaggaaacaccaaagaagacacgtgtgtttttgtcgacaccatcgttcggtgcaacctgcaatcccttgctcagatcgcggagaacgccgccggctgcaagaggtcatcgtcatga

SEQ ID NO: 7 - protein, 227 aa, Vitis vinifera
MPSNPPKSSLVVHRINSPNSITTATTASAAANNHNTSTMPPHKQVQKRSPLTSATQVPDAVSRHHTHVVGPNQCCSAVVQQIAAPVSTVWSVVRRFDNPQAYKHFVKSCHVVVGDGDVGTLREVHVISGLPAANSTERLEILDDERHVLSFSVIGGDHRLSNYRSVTTLHPSPSSTGTVVLESYVVDIPPGNTKEDTCVFVDTIVRCNLQSLAQIAENAAGCKRSSS

SEQ ID NO: 8 - DNA, 666 bp, Populus trichocarpa
atgcctgctaatcctccgaggtcatcccttttaatccatagaatcaacaacaccacaagtaacacaaccctaaacaccaccaacacaaccaccgcaacttcatgccaaaaacggtggtcccctctaccatgcgacgccaccccagttccggagaccgtctcacgctaccacacccacgctgtaggccccaaccagtgctgctctgcggtggtacaacagatcgctgccccaatctccaccgtatggtctgtcgttcgccggttcgacaacccacaagcttataaacatttcgtcaagagctgccacgttatcctcggagacggtgacgtgggcactctccgtgaaatccacgttatctcgggcctcccagctgctcacagcaccgaacgcctcgagatcctcgacgatgaacggcatgtcatcagttttagtgttgttggtggggaccataggcttgcgaattacaagtcggttacaactctccattcgtctccctcagggaacggcaccgtcgtcatggagtcttacgcggtcgatattcctcctggaaatactaaagaggacacgtgtgtgttcgtggataccatagttcggtgcaacctgcagtcactggcacagatcgccgagaactcgaatagacgcaataataaatcatcatcagcgtga

SEQ ID NO: 9 - protein, 221 aa, Populus trichocarpa
MPANPPRSSLLIHRINNTTSNTTLNTTNTTTATSCQKRWSPLPCDATPVPETVSRYHTHAVGPNQCCSAVVQQIAAPISTVWSVVRRFDNPQAYKHFVKSCHVILGDGDVGTLREIHVISGLPAAHSTERLEILDDERHVISFSVVGGDHRLANYKSVTTLHSSPSGNGTVVMESYAVDIPPGNTKEDTCVFVDTIVRCNLQSLAQIAENSNRRNNKSSSA

SEQ ID NO: 10 - DNA, 1308 bp, Solanum lycopersicum
ggctcccactagccccactattttgtcaacctcacgtgaagggtgaacccccatctctttttttttttttaaatacttttctcaacttcaaccaactatttcttaattagctctaatttctacactgtatatatatatacaacttaatcgcaccaataagaacacaagagagaagaaatcatcactactttttcattttctctcaattttcaaatcagcaacaaaaatgcctccaagttcttcagattcatctgttttactccaacgaattagctctaacaatactcatgattttgcttacaagcaatctcatcatcagttacagagacgcatgccgattccctgttcgacggaagtacctgattctgtttcccggtaccatactcacactgtaagtcctgaccagtgctgctccgccgtgatccagcggatctcagctcctgtctccaccgtatggtcggtggtccgccgatttgacaatccgcaggcgtataagcattttgttaaaagctgtcatgttgttgttggcgatggtgacgtcggtactttacgggaagtccgtgtgatttccggtctccctgctgctagtagcacggagaggctagagatcctcgacgacgaacgccacgtcatcagttttagcgttgtcggtggggaccacaagttagccaattaccgatctgtgactacgcttcacacggaaccgagctccggcaatgaagcggcggcggagacgatcgttgtggaatcgtacgtcgttgatgtaccgcctggtaatactagagaagagacgtgcgtttttgtggacacaatcgtgaaatgcaaccttcaatcgttatctcagatcgcgcagaattcagccagatgaaaatcttcgtcaattttgaatatgtattttggatcgatcaacttgctctaacgcttcacttttcgattatattcgtgttgaaaatttcaaaaatatacatttttgaagtatttaatacgtatatattcatattttgaagagttcgaacaggtcgcggatctgaattaatgaagacaactaatcgatgatgcttgtggtgatgatctgatgatatcatatgcgatgcatgattgtttcgttgcacctttattaggtgtgtgttaatctccttgtcatcacttgttgaagataagttcttgatcaaattaatctttgtactttgcttaactaatttcatactctataattttgtctatattgttttctttatcgatttcgttatcatggtatgagacagattcatatatcttaattttaatatgataatttgatttgttttg

SEQ ID NO: 11 - DNA, 642 bp, Solanum lycopersicum
atgcctccaagttcttcagattcatctgttttactccaacgaattagctctaacaatactcatgattttgcttacaagcaatctcatcatcagttacagagacgcatgccgattccctgttcgacggaagtacctgattctgtttcccggtaccatactcacactgtaagtcctgaccagtgctgctccgccgtgatccagcggatctcagctcctgtctccaccgtatggtcggtggtccgccgatttgacaatccgcaggcgtataagcattttgttaaaagctgtcatgttgttgttggcgatggtgacgtcggtactttacgggaagtccgtgtgatttccggtctccctgctgctagtagcacggagaggctagagatcctcgacgacgaacgccacgtcatcagttttagcgttgtcggtggggaccacaagttagccaattaccgatctgtgactacgcttcacacggaaccgagctccggcaatgaagcggcggcggagacgatcgttgtggaatcgtacgtcgttgatgtaccgcctggtaatactagagaagagacgtgcgtttttgtggacacaatcgtgaaatgcaaccttcaatcgttatctcagatcgcgcagaattcagccagatga

SEQ ID NO: 12 - protein, 213 aa, Solanum lycopersicum
MPPSSSDSSVLLQRISSNNTHDFAYKQSHHQLQRRMPIPCSTEVPDSVSRYHTHTVSPDQCCSAVIQRISAPVSTVWSVVRRFDNPQAYKHFVKSCHVVVGDGDVGTLREVRVISGLPAASSTERLEILDDERHVISFSVVGGDHKLANYRSVTTLHTEPSSGNEAAAETIVVESYVVDVPPGNTREETCVFVDTIVKCNLQSLSQIAQNSAR

SEQ ID NO: 13 - DNA, 642 bp, Nicotiana tabacum
atgcctcctagttctccagattcatctgttttactccaaagaataagctccaacactactcctgattttgcctgtaaacaatctcagcaattacaaaggcgtactatgccgataccttgtacgacacaagtcccagattccgttgtccgattccatactcacccagtgggtcccaaccagtgctgctccgccgtgatccagcggatttccgcccccgtctccaccgtatggtcagttgtccgccgcttcgacaacccgcaagcatacaagcatttcgtcaagagctgccacgtcatcgtaggggatggtgacgtcggcactctccgcgaggttcgcgtgatctcaggccttccagctgcgtccagcacggaaagactcgagatcctcgacgacgagcgccatgtcatcagcttcagcgtagtcggcggggaccaccgactcgcgaattaccgttccgtcaccaccctccacccggaaccgtctggtgacgggacgaccatcgtcgttgaatcctacgttgttgatgtaccacctggtaatactagagatgaaacgtgtgttttcgtcgataccatcgtcaaatgcaacctcacatcgttatcgcagatcgcagtaaacgtgaacagaagaaaagattcttga

SEQ ID NO: 14 - protein, 213 aa, Nicotiana tabacum
MPPSSPDSSVLLQRISSNTTPDFACKQSQQLQRRTMPIPCTTQVPDSVVRFHTHPVGPNQCCSAVIQRISAPVSTVWSVVRRFDNPQAYKHFVKSCHVIVGDGDVGTLREVRVISGLPAASSTERLEILDDERHVISFSVVGGDHRLANYRSVTTLHPEPSGDGTTIVVESYVVDVPPGNTRDETCVFVDTIVKCNLTSLSQIAVNVNRRKDS

SEQ ID NO: 15 - DNA, 710 bp, Cucumis sativus
aagtgaaaagctgccatcgccgcggctccgatttcagctatataaatcaggcagcaaaggagaagaaaagagaaaccccacttacacggaaaatgcctcctaatccccctaaatcatccgccgcctcccatcgcataacccactcctccaccgtgccggagtttttcaagcgccagattcaaacacgcgctaccgctgttcctgacgcggtggcgcgttaccacaaccacgctgtttccatgaaccagtgctgctccgccgtcgttcaagagatcgatgccccggtctccaccgtgtggtccgtcgtccgccgcttcgacaacccgcaagcgtacaagcacttcgtcaagagctgtgacgtcatcgtcggtgacggcaacgttggaagcctccgtgaagttcgcgtcatttccggcctcccggcagccaacagcaccgagcggctggagattctcgacgacgaacgtcatattattagcttcagtgtcgtcggcggcgaacaccgactcgctaactaccggtccgttaccactctccacccaaccggcgacggcaccatcgtagtggaatcgtacgtcgtcgacattcctccgggaaacaccgaggaggacacgtgtgtgttcgtcgacaccatcgtccgatgcaaccttcagtcactgactcagatcgctgagaacctgaatcgccggagccgagcagcgccgccgtga

SEQ ID NO: 16 - protein, 205 aa, Cucumis sativus
MPPNPPKSSAASHRITHSSTVPEFFKRQIQTRATAVPDAVARYHNHAVSMNQCCSAVVQEIDAPVSTVWSVVRRFDNPQAYKHFVKSCDVIVGDGNVGSLREVRVISGLPAANSTERLEILDDERHIISFSVVGGEHRLANYRSVTTLHPTGDGTIVVESYVVDIPPGNTEEDTCVFVDTIVRCNLQSLTQIAENLNRRSRAAPP

SEQ ID NO: 17 - DNA, 1161 bp, Glycine max
ctttaacataaaatcacaaaacctatatctttattattattattattattattattattattattgctattattatcacatgaactagaaatgacttctcttcaattccaccgattcaacccagcaaccgatacatccaccgccatcgcaaacggcgtcaactgtccgaagccaccgtcaacgctccgtttattggcgaaagtaagccttagcgtgccggagacggttgctcggcaccacgcgcacccggtggggcccaaccagtgctgctccgtcgtgatccaggcgatcgatgcaccggtctccgccgtctggccggtggtgcggcgcttcgacaacccgcaggcctacaagcacttcgtgaagagctgccacgtggtcgccgcagcaggcggcggcgaggacggcattcgcgtcggcgcgctccgggaggtacgcgtggtctccgggctccccgccgtgtcgagcaccgagcggctcgagatcctcgacgacgagcgccacgtcatgagcttcagcgtcgtcggcggcgaccaccgcctaaggaactaccgctccgtcacgacgctccacggcgacggcaacggagggacagttgtaatcgagtcgtacgtggttgacgtaccgcccggtaacactaaagaagagacttgcgttttcgttgacacaatcgtacggtgcaatttgcagtccctggctcagatcgctgaaacctgaaaatatggcaagccaacaacactcaacattttattttctccgttagtttttttggttattgccttgggtgtttttttgtttcgaatacgggttcgggttcgaaattgatgattacagacaccattaggatttgtttttggaggacttatcaaggtcagacgacaatggttgaaatgaagaatgtgacatataatatatgatatatgattcatgtatatatacatataatatatgtagttctttctatctacctctcttttattttttccattctaatagctgttgagtctgatcgaatctattgttcttgtatccgtggtcaatttatcattaattcatcttgtttgtgagtctgtaatgtagtagagaatgtttctatttgaattaataccatacatatgttgttattagctataggaccaaattagtttacaacttgtcctg

SEQ ID NO: 18 - DNA, 627 bp, Glycine max
atgacttctcttcaattccaccgattcaacccagcaaccgatacatccaccgccatcgcaaacggcgtcaactgtccgaagccaccgtcaacgctccgtttattggcgaaagtaagccttagcgtgccggagacggttgctcggcaccacgcgcacccggtggggcccaaccagtgctgctccgtcgtgatccaggcgatcgatgcaccggtctccgccgtctggccggtggtgcggcgcttcgacaacccgcaggcctacaagcacttcgtgaagagctgccacgtggtcgccgcagcaggcggcggcgaggacggcattcgcgtcggcgcgctccgggaggtacgcgtggtctccgggctccccgccgtgtcgagcaccgagcggctcgagatcctcgacgacgagcgccacgtcatgagcttcagcgtcgtcggcggcgaccaccgcctaaggaactaccgctccgtcacgacgctccacggcgacggcaacggagggacagttgtaatcgagtcgtacgtggttgacgtaccgcccggtaacactaaagaagagacttgcgttttcgttgacacaatcgtacggtgcaatttgcagtccctggctcagatcgctgaaacctga

SEQ ID NO: 19 - protein, 208 aa, Glycine max
MTSLQFHRFNPATDTSTAIANGVNCPKPPSTLRLLAKVSLSVPETVARHHAHPVGPNQCCSVVIQAIDAPVSAVWPVVRRFDNPQAYKHFVKSCHVVAAAGGGEDGIRVGALREVRVVSGLPAVSSTERLEILDDERHVMSFSVVGGDHRLRNYRSVTTLHGDGNGGTVVIESYVVDVPPGNTKEETCVFVDTIVRCNLQSLAQIAET

SEQ ID NO: 20 - DNA, 1145 bp, Fragaria vesca
gcttgttttttcctccttttttgctgatcaaggaccaacttgccttctattttatcatcccacatgaactctcccaagatttaatattcacatttcctcccctttgaaatatacaaccaccccatcttctctcttcttcaaacaatccccaaagctctgctagcttcaagaaactaagctcagagatcatcctctaatgcctcccaacccacccaagtcttctgtattgtgtcaccgcctcaacaccgcccccaacacctcgtctaacaaccagaagaacaacatgatgatcaagaagcagcagcagcagcgcgccccgatcccagaagcggtggcgcgttaccacacgcatggagtcggccctaacaagtgctgctccgccgtgacgcaggagatcgctgctcccgtctccacagtctggtccgtcgtccgccgcttcgataatccccaagcctacaagcacttcgtgaaaagctgccacgttatcgtcggcgacggcgacgttggtaccctccgcgaggttcaggtcatctcggggcttccagccaacaacagcactgagaggctggacgtgctggacgacgagagccatgtcatcagcttcagcatggtcggcggcgaccacaggctgtcaaactacaagtccgtgacgacgcttcacccgtcaccttcgggtaacggaggcacggtggttgtggagtcttacgtggtggacgtgccgccggggaacactaaggaagacacgtgtaacttcgtggacaccatcgtccggtgcaacctgcagtcgctggctcagatcgcagagaatctagcgagacgtaacaacaagtcgtcttcggcgtgccccaagtaataatatgtttatttatttttaattattatgatcggagtattattatggtgatggttattagtaagataacatcttaaattaggtagcaagagtaattagcttgtggtggtgttctgggttttatgtagtctattaggtttcgtcgtcgaacatcaatgtctgcgagagttgtctgactccgagtccgagtccggtctgtgtttgccgttggagggtacggcgtcgttttgtatccgatgaaacagtcacagctgcaagttttatgatgtaatatatatctagtagtgcctta

SEQ ID NO: 21 - protein, 218 aa, Fragaria vesca
MPPNPPKSSVLCHRLNTAPNTSSNNQKNNMMIKKQQQQRAPIPEAVARYHTHGVGPNKCCSAVTQEIAAPVSTVWSVVRRFDNPQAYKHFVKSCHVIVGDGDVGTLREVQVISGLPANNSTERLDVLDDESHVISFSMVGGDHRLSNYKSVTTLHPSPSGNGGTVVVESYVVDVPPGNTKEDTCNFVDTIVRCNLQSLAQIAENLARRNNKSSSACPK

SEQ ID NO: 22 - DNA, 1371 bp, Ricinus communis
gtatgatgcatgataagctcatcttcttcatcatctttcccccaaatatttctctacaattttctctaacaaaagcctcaaactaatccaacttgacacattaaccattttcaagaacaaacctctcttcgtttcaatctctattcatatatatatatatatatatatttttacaaatcctgccatttattacgcatgaatctttaaccctaacttaaatcatatcaagaaactttagccaactcaaaattatgcctgctgcttccctacagctccaaatacccaacactgccaccaccaccacaaccaccacctcggccgccttgtcttgctacaagcattcttggcagccgccagtgcctctttcttgggatgcagccgtgcctgactatgtttcttgctaccatacacgctccgttggtccagatcagtgttgctccgccgtgttcaagatcataaatgcacctgtttccactgtttggtcggtggttcgccgtttcgataacccacaagcttataaacactttgtcaagagctgtcatttaatcaacggtgacggggatgtaggcacgcttagggaggttcacgtggtgtcaggattacctgctgaatcaagtactgaaagactggagattctcgatgatgagcaacatgttataagctttagcatgatcggcggcgatcaccggctaaagaattatcggtcagtgacgactcttcatgcttctccaaatggaaatggtacggttgttattgaatcatatgtagttgatataccagcaggaaatactgaggaggaaacctgtgtttttgttgatacaattttaaggtgcaacttgcaatccttggctcagatagctgagaacatggccaagaattagcaaaatcatcatcatcatcatcatcttgatttgtttacaaacgggtttttcacaactaaaattttttggtttttgattcgggttcggattaattcgatcatctgaattgtcaatgcctgaattgggaatttgtagatccatcagctagcagttcatgaaggtgtttatagtattttgatgctatatatgtcaacttttgttttccttcaagacattgctccctcatttagcatacaattatagtgagattttatgttttacataatgtcttcttgtatttatttattttctttcttcttttttgttaattagttcaagttatttttctcctctttgtttttgttctaaaaggagagaggctcttaattttgtgccttcttgtatatattgtgtctagttccgcctctcttctttctttattatatgtattttcttttggagaaaagaattatattattttatttttgtaattatctcttataatattata

SEQ ID NO: 23 - protein, 209 aa, Ricinus communis
MPAASLQLQIPNTATTTTTTTSAALSCYKHSWQPPVPLSWDAAVPDYVSCYHTRSVGPDQCCSAVFKIINAPVSTVWSVVRRFDNPQAYKHFVKSCHLINGDGDVGTLREVHVVSGLPAESSTERLEILDDEQHVISFSMIGGDHRLKNYRSVTTLHASPNGNGTVVIESYVVDIPAGNTEEETCVFVDTILRCNLQSLAQIAENMAKN

SEQ ID NO: 24 - DNA, 847 bp, Medicago truncatula
gtccttcctcctttgtcctttcaaaaaacctccacttatttaacttctctcctttcttttccaaagacaccaaaaccatcctttcattcattcacattctcaaacctcaaaataaaaaaaaacatcatatcaagtatcaacaacatcatcaaatcaagcactcgagatcacagactaatcttgatcgtcagagtgtttgcaggcacatcatctaaaacacttgatcgtcggtcgcagtgtttgcaggtacaccatctgagcacaagttgttcttgaataacaaaatatgttaccaaacccaacaaccaccgtccccgacgccatcgcccgctaccacacccacgcagtctcccccaaccagtgttgctccgccgtcatccaacatatcgccgcacccgtctccaccgtctggtccgtcgtccgtcgcttcgacaatccacaagcctacaaacacttcgtcaaaagctgccatgtcatcctcggagacggcaacgtcggcactctccgtgaagtccgtgtcatctccggcctccccgccgccgtcagcaccgaacgtctcgaagtcctagacgatgaacgtcatgtcatcagcttcagcatgatcggtggcgatcaccgtcttgctaactaccgttctgtcaccactctccacccttctccgatctccgatgaagatggcaaccaccgctccggcacggtggttgttgagtcctacgttgttgatgttccaccgggaaacaccactgaagatacatgtgtctttgttgatactattcttcggtgcaaccttcaatctctcgcgaaatttgctgagaatttggcttcaacgagatcaaatcaacgataa

SEQ ID NO: 25 - DNA, 561 bp, Medicago truncatula
atgttaccaaacccaacaaccaccgtccccgacgccatcgcccgctaccacacccacgcagtctcccccaaccagtgttgctccgccgtcatccaacatatcgccgcacccgtctccaccgtctggtccgtcgtccgtcgcttcgacaatccacaagcctacaaacacttcgtcaaaagctgccatgtcatcctcggagacggcaacgtcggcactctccgtgaagtccgtgtcatctccggcctccccgccgccgtcagcaccgaacgtctcgaagtcctagacgatgaacgtcatgtcatcagcttcagcatgatcggtggcgatcaccgtcttgctaactaccgttctgtcaccactctccacccttctccgatctccgatgaagatggcaaccaccgctccggcacggtggttgttgagtcctacgttgttgatgttccaccgggaaacaccactgaagatacatgtgtctttgttgatactattcttcggtgcaaccttcaatctctcgcgaaatttgctgagaatttggcttcaacgagatcaaatcaacgataa

SEQ ID NO: 26 - protein, 186 aa, Medicago truncatula
MLPNPTTTVPDAIARYHTHAVSPNQCCSAVIQHIAAPVSTVWSVVRRFDNPQAYKHFVKSCHVILGDGNVGTLREVRVISGLPAAVSTERLEVLDDERHVISFSMIGGDHRLANYRSVTTLHPSPISDEDGNHRSGTVVVESYVVDVPPGNTTEDTCVFVDTILRCNLQSLAKFAENLASTRSNQR

SEQ ID NO: 27 - DNA, 648 bp, Prunus persica
atgccttcctcactgcaattccaaagacccatcaataactctcctaatttttatcccacaaacacattaatccaccacaagcaattccaatcccacgccgcggccgcagctgtagcggaggacttcacgcgcctcaacgctcacgtgatgtcgcccaaccccaaccagtgctgctccgcggttgtgcagtccatcgaggcccccgtcccgaccgtgtggtcggtggtgcggcgcttcgacaacccacaggcctacaagcacttcctcaagagctgccaggtcatcgacgggaccggcgacgtgggcacgctccggaaggtccacgtggtgtcgggcctccccgcggggtccagcacggagcgcctcgagatcctcgacgacgagcgacacgtcctgagcttcagtgttgtgggtggggaccaccggctcgagaactaccgatccgtcaccacgctccacgactcgccgagtggactcgggaccgtcgtggtggagtcgtacgtggtggacgttccgccgggaaacaccaaggaggagacgtgcgtgttcgtcgacaccatcgtgcgctgcaacttgcagtcgctggctcagatcgcggagagcatggccaaaccatccaccaagagtaacaacaacaagccctcatga

SEQ ID NO: 28 - protein, 215 aa, Prunus persica
MPSSLQFQRPINNSPNFYPTNTLIHHKQFQSHAAAAAVAEDFTRLNAHVMSPNPNQCCSAVVQSIEAPVPTVWSVVRRFDNPQAYKHFLKSCQVIDGTGDVGTLRKVHVVSGLPAGSSTERLEILDDERHVLSFSVVGGDHRLENYRSVTTLHDSPSGLGTVVVESYVVDVPPGNTKEETCVFVDTIVRCNLQSLAQIAESMAKPSTKSNNNKPS

SEQ ID NO: 29 - DNA, 940 bp, Hordeum vulgare
gagctaatcctaagttcccaacccacccactctcctaaaatttcttcttcacagcgataaagctcagaagctcgagccgcccgcggcttgtctacaatgccgtacgcagctgcacggccgtcgccccagcagcacagccggatcagcgccgcctgtaaggcgttggtggcgcagggtgcagcggtgccgggcgaggtggcgcggcaccacgagcacgcggccggcgcggggcagtgctgctcggccgtggtgcaggcgatcgcggcgcccgtggaggcggtgtggtcggtcgtgcggcgcttcgaccggccgcaggcgtacaagcgcttcatcaagagctgccgcctggtggacggcgacggtggcgcggtgggatcggtgcgggaggtgcgcgtcgtctccggtctgcctggcaccagcagccgcgagcggctcgagatcctggacgacgagcggcgcgtgctcagcttccggatcgtcggcggcgagcaccgcctcgccaactaccggtccgtcaccaccgtgaacgaggtggcgtcgacggtggccggggcgccgcgggtgaccctggtggtcgagtcttatgtggtggacgtgccgccggggaacacgggggacgagacgcgcatgttcgtggacaccatcgtgcggtgcaacctccagtcgctcgcgcgcacggcggagcaactcgcgctggcagcgccgcgcgtgaactgatgcccgtcatgcactccggcagaccagctgaaaggtaccaccgtagacccgtaccacgggcgcaaggtcgaggttctggaatctggagtgacatgatttacacccatgcattagccctagtgtcatgtgaagaaagattggctttgtgcacctggctgttttgtgtaaatcatctcatcgtgttaacttagtagttgaattaatgtgcagtactgattggatt

SEQ ID NO: 30 - DNA, 618 bp, Hordeum vulgare
atgccgtacgcagctgcacggccgtcgccccagcagcacagccggatcagcgccgcctgtaaggcgttggtggcgcagggtgcagcggtgccgggcgaggtggcgcggcaccacgagcacgcggccggcgcggggcagtgctgctcggccgtggtgcaggcgatcgcggcgcccgtggaggcggtgtggtcggtcgtgcggcgcttcgaccggccgcaggcgtacaagcgcttcatcaagagctgccgcctggtggacggcgacggtggcgcggtgggatcggtgcgggaggtgcgcgtcgtctccggtctgcctggcaccagcagccgcgagcggctcgagatcctggacgacgagcggcgcgtgctcagcttccggatcgtcggcggcgagcaccgcctcgccaactaccggtccgtcaccaccgtgaacgaggtggcgtcgacggtggccggggcgccgcgggtgaccctggtggtcgagtcttatgtggtggacgtgccgccggggaacacgggggacgagacgcgcatgttcgtggacaccatcgtgcggtgcaacctccagtcgctcgcgcgcacggcggagcaactcgcgctggcagcgccgcgcgtgaac

SEQ ID NO: 31 - protein, 206 aa, Hordeum vulgare
MPYAAARPSPQQHSRISAACKALVAQGAAVPGEVARHHEHAAGAGQCCSAVVQAIAAPVEAVWSVVRRFDRPQAYKRFIKSCRLVDGDGGAVGSVREVRVVSGLPGTSSRERLEILDDERRVLSFRIVGGEHRLANYRSVTTVNEVASTVAGAPRVTLVVESYVVDVPPGNTGDETRMFVDTIVRCNLQSLARTAEQLALAAPRVN

SEQ ID NO: 32 - DNA, 1228 bp, Zea mays
ctttccacacataatgagcatacctacaagccatattcccatccacattcatcccccaacacccactcgtccacccaccgttttccttgtcttcactttcctcacgcaattcacccaccccctcaccatttcccccatcgaccattgctgccacataccaaacccacttcctgtggtcttcggtgtcttccccgccgccgccgctgccgtcgccgtcgtgcatcacactgtccctcttgctgtgatatctatgagagagagaaacagctcgatcgatcaagaacaccaacgaggctctagctccagatcgacaatgccgttcgcagcctcaaggacgtcacagcagcagcacagccgtgtggccaccaacgggagggccgtggcggtgtgcgcgggtcacgcgggcgtgcccgacgaggtggcgcggcaccacgagcacgctgtggcagcggggcaatgctgcgccgccatggtgcagtccatcgcagcgccggtggacgcggtgtggtcgctggtgcgtcgcttcgaccagccgcagcggtacaagcgcttcatcaggagctgccacctcgtggacggcgacggcgccgaggtggggtccgtgcgggagctcctgctcgtgtccgggctgcccgccgagagcagccgcgagcggcttgagatccgggacgacgagcggcgggtgatcagcttccgggtcctgggcggcgaccaccgcctggccaactaccgctccgtgaccaccgtgcacgaggcggcgccgtcgcaggacgggcgcccgctcaccatggtcgtcgagtcctacgtggtggacgtgccgccggggaacaccgtcgaggagacgcgcatcttcgtggacaccatcgttcggtgcaacctccagtccctcgagggcacggtcatcaggcagctggagatcgcggcaatgccgcacgacgacaaccagaactgattgccgtcgggccggccggctgcacaccgtcatcgcagttgggctgccggtttatttggacgaggcttcatcgaccatttatgatatattttctttcgtcgttggtactgtatactctcgcgaaaataagaagttggtactgtgaactgcttaaagtttgaggttgtttgtcttccctttcttaaactctgccaataatgaagtattgtaagttacagcatcaataatattaagaccagtaaagtagtatagttaaaaaaaaaaaaaaaaaaaaaa

SEQ ID NO: 33 - DNA, 1207 bp, Zea mays
gttgagtggaaaaccatataatctgacaccttcgccacctctgtctcatcaccttttgttgttgcctctaccattagagaccatacccctaccttagtagctatcagatctgtttcccccgacccactcaaaatattgaaccactacttccaatccctaaacctaatgacacctgagcgctacccatcataggatcgccatcatccattgaataaaatagaagagagaaacaactcgatcgatcgatcaagaagaccggcgaggctctagctccagaccgacaatgccgtacgcagccacaaggacgtcgccgcagcagcacagccgcgtggccagcaacgggagggccgtggcggcgtgcgcaggccacgcgggcgtgccggacgaggtggcgcggcaccacgagcacgcggtggccgcggggcagtgctgctctgtgatggtgcagtccatcgcggcgccggcggacgcggtgtggtcgctggtgcgccgcttcgaccagccgcaggggtacaagcgcttcatcaggagctgccacctggtggacggcgacggcgtcgaggtggggtccgtgcgggagctcctggtcgtgtccgggctgcccgccgagaacagccgcgagcggcttgagatccgggacgacgagcggcgggtgatcagcttccggatcctgggcggcgaccaccgcctcgccaactaccgctccgtgaccaccgtgcacgaggcggcctcagagggcgggccgctcaccatggtcgtcgagtcctatgtggtggacgtgccgccggggaacaccgtcgaggagacgcgcatcttcgtggacaccatcgttcggtgcaacctccaatccctcgaggacacggttatcaggcagcaggcgatggcggcaccggcagcgccgcacaacgaccacaaccacagctgatcgccgccgggcccggccggctggacaccctcgcagtgggggctgccggtttcttcggaggcttcatctgccatttatgatgttttctttcgccgttggtattctatagtttcaccaataagaagaataggtactgtgaactttttaaaggttgaggttgttcgtcttccctttcttaaactctaaaaacgacaataaagtattgtatgatacaccaccaataatattaagaccagtaaagtagtatagttctaatatatatgtgtaagtttgtgtatagat

SEQ ID NO: 34 - protein, 213 aa, Zea mays
MPYAATRTSPQQHSRVASNGRAVAACAGHAGVPDEVARHHEHAVAAGQCCSVMVQSIAAPADAVWSLVRRFDQPQGYKRFIRSCHLVDGDGVEVGSVRELLVVSGLPAENSRERLEIRDDERRVISFRILGGDHRLANYRSVTTVHEAASEGGPLTMVVESYVVDVPPGNTVEETRIFVDTIVRCNLQSLEDTVIRQQAMAAPAAPHNDHNHS

SEQ ID NO: 35 - DNA, 1173 bp, Oryza sativa
caacctcctcatcgccgcatagaaattccaccgaacacatagcgagcaagaagaggaggaggaggagaagcgaaggagacaaggaaacagcgcgacatgccgtgcatcccggcgtccagccctggcatcccgcaccagcaccagcaccagcaccaccgggcgctagcaggcgtcggcatggcggtcgggtgcgcggcggaggcggccgtggccgcggcgggtgtcgcggggacgaggtgcggggcgcacgacggggaggtgcctatggaggtggcgcggcaccacgagcacgcggagccagggtcggggcggtgctgctccgcggtggtccagcacgtagcggcgccggcggcggcggtgtggtcggtggtgcggcggttcgaccagccccaggcgtacaagcggttcgtccgcagctgcgcgctgctcgccggggacggcggcgtgggcacgctccgcgaggtgcgcgtcgtgtcgggcctccccgcggcgtcctcccgcgagcgcctcgagatcctcgacgacgagagccacgtcctcagcttccgcgtcgtcggcggcgagcaccgcctcaagaactacctctcggtcaccaccgtccacccgtccccgtccgcgccgacggccgccaccgtcgtggtggagtcctacgtcgtcgacgtgcccccgggcaacacgcccgaggacacccgcgtgttcgtcgacaccatcgtcaagtgcaacctccagtctctcgccaagaccgccgagaagctcgccgccggcgcgagggccgccggctcgtgagcgctctccaccgccgggccaccgatcgtttttttccgggcggatggatgtgtgtttcttcggcgcgtgagagctttctcctttttttccccctttttcctctttggtttcttcacatttgaccccctcttttttttcctctttatttttttccctcgttgttgcgagagacacacaaacaccatccacccttgtccaaaaagattcaagaaatgacatgtataattgacagcctgcaattccatttgtatgctcgagttggttggtgtattgagtacatacatacatatacataaacacgatagtagtattagatatttagatctgttctctggcacaaatttaaagtgtacgttcgtatttaaagttgtgctacatctatacagc

SEQ ID NO: 36 - DNA, 660 bp, Oryza sativa
atgccgtgcatcccggcgtccagccctggcatcccgcaccagcaccagcaccagcaccaccgggcgctagcaggcgtcggcatggcggtcgggtgcgcggcggaggcggccgtggccgcggcgggtgtcgcggggacgaggtgcggggcgcacgacggggaggtgcctatggaggtggcgcggcaccacgagcacgcggagccagggtcggggcggtgctgctccgcggtggtccagcacgtagcggcgccggcggcggcggtgtggtcggtggtgcggcggttcgaccagccccaggcgtacaagcggttcgtccgcagctgcgcgctgctcgccggggacggcggcgtgggcacgctccgcgaggtgcgcgtcgtgtcgggcctccccgcggcgtcctcccgcgagcgcctcgagatcctcgacgacgagagccacgtcctcagcttccgcgtcgtcggcggcgagcaccgcctcaagaactacctctcggtcaccaccgtccacccgtccccgtccgcgccgacggccgccaccgtcgtggtggagtcctacgtcgtcgacgtgcccccgggcaacacgcccgaggacacccgcgtgttcgtcgacaccatcgtcaagtgcaacctccagtctctcgccaagaccgccgagaagctc

SEQ ID NO: 37 - protein, 229 aa, Oryza sativa
MPCIPASSPGIPHQHQHQHHRALAGVGMAVGCAAEAAVAAAGVAGTRCGAHDGEVPMEVARHHEHAEPGSGRCCSAVVQHVAAPAAAVWSVVRRFDQPQAYKRFVRSCALLAGDGGVGTLREVRVVSGLPAASSRERLEILDDESHVLSFRVVGGEHRLKNYLSVTTVHPSPSAPTAATVVVESYVVDVPPGNTPEDTRVFVDTIVKCNLQSLAKTAEKLAAGARAAGS

SEQ ID NO: 38 - DNA, 228 bp, Triticum aestivum
cgcgagcggctcgagatcctggacgacgagagccacgtgctcagtttccgcgtcgtcggtggcgagcaccggctcaagaactacctctccgtcaccaccgtccacccgtccccggccgcgccgtccagcgccaccgtcgtcgtggagtcgtacgtcgtggacgtgcccgcgggcaacacgaccgaggacacccgcgtgttcatcgacaccatcgtcaagtgcaacctc

SEQ ID NO: 39 - protein, 76 aa, Triticum aestivum
RERLEILDDESHVLSFRVVGGEHRLKNYLSVTTVHPSPAAPSSATVVVESYVVDVPAGNTTEDTRVFIDTIVKCNL

SEQ ID NO: 40 - DNA, 701 bp, Citrus sinensis
cgtcgcttcgacaacccccaggcttacaagcacttcgtcaagagctgccacgtcattaacggcgacggcgacgtgggcaccctccgggaggtccacgtcatctccggactccccgccggccgcagcacggagaggctcgagatcctagacgacgagcgccacgtcataagcttcagcgtcatcgggggcgaccacaggctggcgaattacaggtcggtgacgactctccacccatcgcccgctggcaacgggacggtggtcgtggagtcttacgtcgtcgacgtgcctcccggaaacacgaacgaagacacctgcgtgttcgtcgacaccatcgttaaatgcaacctccagtcgctggcgcagaccgcggagaatctgaccaggcgaaacaacaacaaccacagcagtaacaacagcagcatcagcagcaacaacaacggcccgatcaggtcatgttctgttctcgagtgaatagttctagttactagtattacaaaatagtgttgatgtctttggtaatacgcagtcgtttttgtgagtatccgatgtggattgcctaaagaaactacagaatcatctcgtttctgttaactacttcttcatcatgttcatttggttactgcatttttcttttttcttttttttttaaattattaatgtgaaatatttttaattgataaataataatgtcgacctacagc

SEQ ID NO: 41 - DNA, 720 bp, Citrus sinensis
atgccagtaaatccaccgaaatcatctctattgctgcacagaatcaacaacgtaaacacagccaccaacacgatcgcaacagcaacagccaacatgctttgccagaaagagcagcttcagttccaaaagcgcttcccagcaacgtggtcaacccccgtccccgacgccgtggcacgccaccacaccctcgtcgttggccccaaccagtgctgctcctccgtcgtccagcagatcgccgctcccgtctcgaccgtctggtccgtcgtccgtcgcttcgacaacccccaggcttacaagcacttcgtcaagagctgccacgtcattaacggcgacggcgacgtgggcaccctccgggaggtccacgtcatctccggactccccgccggccgcagcacggagaggctcgagatcctagacgacgagcgccacgtcataagcttcagcgtcatcgggggcgaccacaggctggcgaattacaggtcggtgacgactctccacccatcgcccgctggcaacgggacggtggtcgtggagtcttacgtcgtcgacgtgcctcccggaaacacgaacgaagacacctgcgtgttcgtcgacaccatcgttaaatgcaacctccagtcgctggcgcagaccgcggagaatctgaccaggcgaaacaacaacaaccacagcagtaacaacagcagcatcagcagcaacaacaacggcccgatcaggtca

SEQ ID NO: 42 - protein, 245 aa, Citrus sinensis
MPVNPPKSSLLLHRINNVNTATNTIATATANMLCQKEQLQFQKRFPATWSTPVPDAVARHHTLVVGPNQCCSSVVQQIAAPVSTVWSVVRRFDNPQAYKHFVKSCHVINGDGDVGTLREVHVISGLPAGRSTERLEILDDERHVISFSVIGGDHRLANYRSVTTLHPSPAGNGTVVVESYVVDVPPGNTNEDTCVFVDTIVKCNLQSLAQTAENLTRRNNNNHSSNNSSISSNNNGPIRSCSVLE

SEQ ID NO: 43 - protein, 221 aa, Arabidopsis thaliana
MANSESSSSPVNEEENSQRISTLHHQTMPSDLTQDEFTQLSQSIAEFHTYQLGNGRCSSLLAQRIHAPPETVWSVVRRFDRPQIYKHFIKSCNVSEDFEMRVGCTRDVNVISGLPANTSRERLDLLDDDRRVTGFSITGGEHRLRNYKSVTTVHRFEKEEEEERIWTVVLESYVVDVPEGNSEEDTRLFADTVIRLNLQKLASITEAMNRNNNNNNSSQVR

SEQ ID NO: 44 - protein, 190 aa, Arabidopsis thaliana
MSSSPAVKGLTDEEQKTLEPVIKTYHQFEPDPTTCTSLITQRIHAPASVVWPLIRRFDNPERYKHFVKRCRLISGDGDVGSVREVTVISGLPASTSTERLEFVDDDHRVLSFRVVGGEHRLKNYKSVTSVNEFLNQDSGKVYTVVLESYTVDIPEGNTEEDTKMFVDTVVKLNLQKLGVAATSAPMHDDE

SEQ ID NO: 45 - protein, 209 aa, Arabidopsis thaliana
MNLAPIHDPSSSSTTTTSSSTPYGLTKDEFSTLDSIIRTHHTFPRSPNTCTSLIAHRVDAPAHAIWRFVRDFANPNKYKHFIKSCTIRVNGNGIKEIKVGTIREVSVVSGLPASTSVEILEVLDEEKRILSFRVLGGEHRLNNYRSVTSVNEFVVLEKDKKKRVYSVVLESYIVDIPQGNTEEDTRMFVDTVVKSNLQNLAVISTASPT

SEQ ID NO: 46 - protein, 203 aa, Arabidopsis thaliana
MRSPVQLQHGSDATNGFHTLQPHDQTDGPIKRVCLTRGMHVPEHVAMHHTHDVGPDQCCSSVVQMIHAPPESVWALVRRFDNPKVYKNFIRQCRIVQGDGLHVGDLREVMVVSGLPAVSSTERLEILDEERHVISFSVVGGDHRLKNYRSVTTLHASDDEGTVVVESYIVDVPPGNTEEETLSFVDTIVRCNLQSLARSTNRQ

SEQ ID NO: 47 - protein, 215 aa, Arabidopsis thaliana
MPTSIQFQRSSTAAEAANATVRNYPHHHQKQVQKVSLTRGMADVPEHVELSHTHVVGPSQCFSVVVQDVEAPVSTVWSILSRFEHPQAYKHFVKSCHVVIGDGREVGSVREVRVVSGLPAAFSLERLEIMDDDRHVISFSVVGGDHRLMNYKSVTTVHESEEDSDGKKRTRVVESYVVDVPAGNDKEETCSFADTIVRCNLQSLAKLAENTSKFS

SEQ ID NO: 48 - protein, 211 aa, Arabidopsis thaliana
MEMIGGDDTDTEMYGALVTAQSLRLRHLHHCRENQCTSVLVKYIQAPVHLVWSLVRRFDQPQKYKPFISRCTVNGDPEIGCLREVNVKSGLPATTSTERLEQLDDEEHILGINIIGGDHRLKNYSSILTVHPEMIDGRSGTMVMESFVVDVPQGNTKDDTCYFVESLIKCNLKSLACVSERLAAQDITNSIATFCNASNGYREKNHTETNL

SEQ ID NO: 49 - protein, 188 aa, Arabidopsis thaliana
MEANGIENLTNPNQEREFIRRHHKHELVDNQCSSTLVKHINAPVHIVWSLVRRFDQPQKYKPFISRCVVKGNMEIGTVREVDVKSGLPATRSTERLELLDDNEHILSIRIVGGDHRLKNYSSIISLHPETIEGRIGTLVIESFVVDVPEGNTKDETCYFVEALIKCNLKSLADISERLAVQDTTESRV

SEQ ID NO: 50 - protein, 187 aa, Arabidopsis thaliana
MMDGVEGGTAMYGGLETVQYVRTHHQHLCRENQCTSALVKHIKAPLHLVWSLVRRFDQPQKYKPFVSRCTVIGDPEIGSLREVNVKSGLPATTSTERLELLDDEEHILGIKIIGGDHRLKNYSSILTVHPEIIEGRAGTMVIESFVVDVPQGNTKDETCYFVEALIRCNLKSLADVSERLASQDITQ

SEQ ID NO: 51 - protein, 183 aa, Arabidopsis thaliana
MNGDETKKVESEYIKKHHRHELVESQCSSTLVKHIKAPLHLVWSIVRRFDEPQKYKPFISRCVVQGKKLEVGSVREVDLKSGLPATKSTEVLEILDDNEHILGIRIVGGDHRLKNYSSTISLHSETIDGKTGTLAIESFVVDVPEGNTKEETCFFVEALIQCNLNSLADVTERLQAESMEKKI

SEQ ID NO: 52 - protein, 161 aa, Arabidopsis thaliana
METSQKYHTCGSTLVQTIDAPLSLVWSILRRFDNPQAYKQFVKTCNLSSGDGGEGSVREVTVVSGLPAEFSRERLDELDDESHVMMISIIGGDHRLVNYRSKTMAFVAADTEEKTVVVESYVVDVPEGNSEEETTSFADTIVGFNLKSLAKLSERVAHLKL

SEQ ID NO: 53 - protein, 159 aa, Arabidopsis thaliana
MKTSQEQHVCGSTVVQTINAPLPLVWSILRRFDNPKTFKHFVKTCKLRSGDGGEGSVREVTVVSDLPASFSLERLDELDDESHVMVISIIGGDHRLVNYQSKTTVFVAAEEEKTVVVESYVVDVPEGNTEEETTLFADTIVGCNLRSLAKLSEKMMELT

SEQ ID NO: 54 - protein, 148 aa, Arabidopsis thaliana
MESSKQKRCRSSVVETIEAPLPLVWSILRSFDKPQAYQRFVKSCTMRSGGGGGKGGEGKGSVRDVTLVSGFPADFSTERLEELDDESHVMVVSIIGGNHRLVNYKSKTKVVASPEDMAKKTVVVESYVVDVPEGTSEEDTIFFVKMMK

SEQ ID NO: 55 - DNA, 624 bp, Arabidopsis thaliana
atggttgccgttcaccgtccttcttccgccgtatcagacggagattccgttcagattccgatgatgatcgcgtcgtttcaaaaacgttttccttctctctcacgcgactccacggccgctcgttttcacacacacgaggttggtcctaatcagtgttgctccgccgttattcaagagatctccgctccaatctccaccgtttggtccgtcgtacgccgctttgataacccacaagcttacaaacactttctcaaaagctgtagcgtcatcggcggagacggcgataacgttggtagcctccgtcaagtccacgtcgtctctggtctccccgccgctagctccaccgagagactcgatatcctcgacgacgaacgccacgtcatcagcttcagcgttgttggtggtgaccaccggctctctaactaccgatccgtaacgacccttcacccttctccgatctccgggaccgtcgttgtcgagtcttacgtcgttgatgttcctccaggcaacacaaaggaagagacttgtgacttcgttgacgttatcgtacgatgcaatcttcaatctcttgcgaaaataactgagaatactgcggctgagagcaagaagaagatgtctctgtga

SEQ ID NO: 56 - DNA, 624 bp, Arabidopsis thaliana
atggttgccgttcaccgtccttcttccgccgtatcagacggagattccgttcagattccgatgatgatcgcgtcgtttcaaaaacgttttccttctctctcacgcgactccacggccgctcgttttcacacacacgaggttggtcctaatcagtgttgctccgccgttattcaagagatctccgctccaatctccaccgtttggtccgtcgtacgccgctttgataacccacaagcttacaaacgctttctcaaaagctgtagcgtcatcggcggagacggcgataacgctggtagcctccgtcaagtccacgtcgtctctggtctccccgccgctagctccaccgagagactcgatatcctcgacgacgaacgccacgtcatcagcttcagcgttgttggtggtgaccaccggctctctaactaccgatccgtaacgacccttcacccttctccgatctccgggaccgtcgttgtcgagtcttacgtcgttgatgttcctccaggcaacacaaaggaagagacttgtgacttcgttgacgttatcgtacgatgcaatcttcaatctcttgcgaaaatagccgagaatactgcggctgagagcaagaagaagatgtctctgtga

SEQ ID NO: 57 - protein, 5 aa, Arabidopsis thaliana
SGLPA

SEQ ID NO: 58 - protein, 5 aa, Arabidopsis thaliana
GDHRL

SEQ ID NO: 59 - protein, 26 aa, Arabidopsis thaliana
EETCDFVDVIVRCNLQSLAKIAENTA

SEQ ID NO: 60 - protein, 207 aa, Arabidopsis thaliana
MLAVHRPSSAVSDGDSVQIPMMIASFQKRFPSLSRDSTAARFHTHEVGPNQCCSAVIQEISAPISTVWSVVRRFDNPQAYKHFLKSCSVIGGDGDNVGSLRQVHVVSGLPAASSTERLDILDDERHVISFSVVGGDHRLSNYRSVTTLHPSPISGTVVVESYVVDVPPGNTKEETCDFVDVIVRCNLQSLAKITENTAAESKKKMSL

SEQ ID NO: 61 - protein, 207 aa, Arabidopsis thaliana
MLAVHRPSSAVSDGDSVQIPMMIASFQKRFPSLSRDSTAARFHTHEVGPNQCCSAVIQEISAPISTVWSVVRRFDNPQAYKHFLKSCSVIGGDGDNAGSLRQVHVVSGLPAASSTERLDILDDERHVISFSVVGGDHRLSNYRSVTTLHPSPISGTVVVESYVVDVPPGNTKEETCDFVDVIVRCNLQSLAKIAENTAAESKKKMSL

SEQ ID NO: 62 - protein, 207 aa, Arabidopsis thaliana
MLAVHRPSSAVSDGDSVQIPMMIASFQKRFPSLSRDSTAARFHTHEVGPNQCCSAVIQEISAPISTVWSVVRRFDNPQAYKRFLKSCSVIGGDGDNAGSLRQVHVVSGLPAASSTERLDILDDERHVISFSVVGGDHRLSNYRSVTTLHPSPISGTVVVESYVVDVPPGNTKEETCDFVDVIVRCNLQSLAKIAENTAAESKKKMSL

SEQ ID NO: 63 - protein, 207 aa, Arabidopsis thaliana
MLAVHRPSSAVSDGDSVQIPMMIASFQKRFPSLSRDSTAARFHTHEVGPNQCCSAVIQEISAPISTVWSVVRRFDNPQAYKHFLKSCSVIGGDGDNVGSLRQVHVVSGLPAASSTERLDILDDERHVISYSVVGGDHRLSNYRSVTTLHPSPISGTVVVESYVVDVPPGNTKEETRDFVDVIVRCNLQSLAKIAENTAAESKKKMSL

SEQ ID NO: 64 - protein, 207 aa, Arabidopsis thaliana
MLAVHRPSSAVSDGDSVQIPMMIASFQKRFPSLSRDSTAARFHTHEVGPNQCCSAVIQEISAPISTVWSVVRRFDNPQAYKHFLKSCSVIGGDGDNVGSLRQVHVVSGLPAASSTERLDILDDERHVISYSVVGGDHRLSNYRSVTTLHPSPISGTVVVESYVVDVPPGNTKEETCDFVDVIVRCNLQSLAKIAENTAAESKKKMSL

SEQ ID NO: 65 - protein, 207 aa, Arabidopsis thaliana
MLAVHRPSSAVSDGDSVQIPMMIASFQKRFPSLSRDSTAARFHTHEVGPNQCCSAVIQEISAPISTVWSVVRRFDNPQAYKHFLKSCSVIGGDGDNVGSLRQVHVVSGLPAASSTERLDILDDERHVISFSVVGGDHRLSNYRSVTTLHPSPISGTVVVESYVVDVPPGNTKEETRDFVDVIVRCNLQSLAKIAENTAAESKKKMSL

SEQ ID NO: 66 - DNA, 33 bp, Artificial Sequence
  Primer
gcagcagccatggttgccgttcaccgtccttct

SEQ ID NO: 67 - DNA, 30 bp, Artificial Sequence
  Primer
cgcacgaattcacagagacatcttcttctt

SEQ ID NO: 68 - protein, 191 aa, Arabidopsis thaliana
MPSELTPEERSELKNSIAEFHTYQLDPGSCSSLHAQRIHAPPELVWSIVRRFDKPQTYKHFIKSCSVEQNFEMRVGCTRDVIVISGLPANTSTERLDILDDERRVTGFSITGGEHRLTNYKSVTTVHRFEKENRIWTVVLESYVVDMPEGNSRDDTRMFADTVVKLNLQKLATVAEAMARNSGDGSGSQVT

SEQ ID NO: 69 - protein, 221 aa, Arabidopsis thaliana; identical to SEQ ID NO: 43

SEQ ID NO: 70 - protein, 190 aa, Arabidopsis thaliana; identical to SEQ ID NO: 44

SEQ ID NO: 71 - protein, 207 aa, Arabidopsis thaliana; identical to SEQ ID NO: 3

(19) United States
(12) Patent Application Publication
(10) Pub. No.: US 2016/0068860 A1
(43) Pub. Date: Mar. 10, 2016

(54) TRANSGENIC PLANTS

(71) Applicants: CONSEJO SUPERIOR DE INVESTIGACIONES CIENTIFICAS (CSIC), Madrid (ES); UNIVERSIDAD POLITÉCNICA DE VALENCIA, Valencia (ES)

(72) Inventor: Pedro Luis Rodriguez Egea, Valencia (ES)

(21) Appl. No.: 14/891,260

(22) PCT Filed: May 13, 2014
(86) PCT No.: PCT/EP2014/059772

§ 371 Date: Nov. 13, 2015

(30) Foreign Application Priority Data
May 13, 2013 (EP) 13382177.7

(51) Int. Cl. C12N 15/82; C07K 14/415

(52) U.S. Cl. CPC C12N 15/8293; C12N 15/8271; C12N 15/8273; C12N 15/8267; C07K 14/415

(57) ABSTRACT

The invention relates to a method for manipulation of the ABA signalling pathway and transgenic plants with improved stress resistance.

### FIELD OF THE INVENTION

The invention relates to transgenic plants with improved phenotypic traits, including enhanced stress resistance. The improved traits are conferred by enhanced ABA receptor signalling. Also within the scope of the invention are related methods, uses, isolated nucleic acids and vector constructs.

### INTRODUCTION

The ever-increasing world population and the dwindling supply of arable land available for agriculture fuels research towards increasing the efficiency of agriculture. Conventional means for crop and horticultural improvements utilise selective breeding techniques to identify plants having desirable characteristics. However, such selective breeding techniques have several drawbacks, namely that these techniques are typically labour intensive and result in plants that often contain heterogeneous genetic components that may not always result in the desirable trait being passed on from parent plants. Advances in molecular biology have allowed mankind to modify the germplasm of animals and plants. Genetic engineering of plants entails the isolation and manipulation of genetic material (typically in the form of DNA or RNA) and the subsequent introduction of that genetic material into a plant. Alternatively methods can be used to modify or change, or “edit”, the existing genetic material in a targeted manner, altering just one or a few amino acids of the encoded protein, for example using mutagenesis or CRISPR technology. Such technology has the capacity to deliver crops or plants having various improved economic, agronomic or horticultural traits. Traits of particular economic interest are growth and stress resistance, as these are determinants of eventual crop yield.

Plants adapt to changing environmental conditions by modifying their growth. Plant growth and development is a complex process involves the integration of many environmental and endogenous signals that, together with the intrinsic genetic program, determine plant form. Factors that are involved in this process include several growth regulators collectively called the plant hormones or phytohormones. This group includes auxin, cytokinin, the gibberellins (GAs), abscisic acid (ABA), ethylene, the brassinosteroids (BRs), and jasmonic acid (JA), each of which acts at low concentrations to regulate many aspects of plant growth and development. Abiotic and biotic stress can negatively impact on plant growth leading to significant losses in agriculture. Even moderate stress can have significant negative impact on plant growth and thus reduce the yield of agriculturally important crop plants. In any given season or location, crops very commonly experience periods of moderate stress or one kind or another, which restricts the productivity of that crop. Therefore, finding a way to improve growth, in particular under stress conditions, is of great economic interest.

ABA plays a critical role both for plant biotic and abiotic stress response (Cutler et al., 2010). Since ABA is recognized as the critical hormonal regulator of plant response to water stress, both the ABA biosynthetic and signalling pathways can be considered as potential targets to improve plant performance under drought. Thus, it has been demonstrated that transgenic plants producing high levels of ABA display improved growth under drought stress compared to wild type (Iuchi et al. 2001; Qin & Zeevaart 2002). Priming of ABA biosynthesis can be obtained by direct over-expression of 9-cisepoxycarotenoid dioxygenase, a key enzyme in the biosynthetic pathway (Iuchi et al. 2001; Qin & Zeevaart 2002), or through the use of chemicals that accelerate ABA accumulation (Jakab et al., 2005). On the other hand, some examples are also known of Arabidopsis mutants (era1, abh1, pp2c combined mutants) affected in ABA signal transduction that show both an enhanced ABA response and drought resistant phenotypes (Pei et al., 1998; Hugouvieux et al., 2001; Saez et al., 2006). For instance, enhancement of abscisic acid sensitivity and reduction of water consumption has been achieved in Arabidopsis by combined inactivation of the protein phosphatases type 2C (PP2Cs) ABI1 and HAB1, leading to drought resistant plants (Saez et al., 2006).

Enhancing ABA signaling through the recently discovered PYR/PYL ABA receptors is another approach to improve plant drought resistance, for instance through over-expression of the receptors or generation of constitutively active versions (Santiago et al., 2009; Xaavedra et al., 2009; Mosquna et al., 2011, WO 2013/006263). However, pleiotropic effects due to sustained effects of high ABA levels or active ABA signalling might negatively affect plant growth, since abiotic stress responses divert resources required for normal growth. In Mosquna et al., 2011 and WO 2013/006263, constitutively active ABA mutant PYR1, PYL2 and PYL9 polypeptides are disclosed which inhibit PP2C in the absence of ABA. It was shown that only the combination of several specific amino acid substitutions was sufficient to convey the desired effects and produce variants with activation levels nearly indistinguishable from ABA saturation wild type PYR1.

Recent studies reveal at least two subclasses of PYR/PYL receptors, including monomeric and dimeric PYLs (Dupeux et al., 2011a; Hao et al., 2011). The dimeric receptors show a higher Kd for ABA (>50 μM, lower affinity) than monomeric ones (˜1 μM); however, in the presence of certain clade A protein phosphatases 2C (PP2Cs), both groups of receptors form ternary complexes with high affinity for ABA (Kd 30-60 nM) (Ma et al., 2009; Santiago et al., 2009a, b). A third subclass appears when we consider the trans-dimeric PYL3 receptor, which suffers a cis- to trans-dimer transition upon ligand binding to facilitate the posterior dissociation to monomer (Zhang et al., 2012). Dimeric receptors occlude their surface of interaction with the PP2C in the dimer, so they are strongly ABA-dependent for dissociation and adoption of a PP2C binding conformation (Dupeux et al., 2011). In vitro, monomeric ABA receptors are able to interact in the absence of ABA to some extent with the catalytic core of PP2Cs, although less stable complexes are formed compared to ternary complexes with ABA (Dupeux et al., 2011; Hao et al., 2011). In planta, tandem affinity purification (TAP) and mass spectrometrical analysis of PYL8-interacting partners was largely dependent on ABA to recover PYL8-PP2C complexes (Antoni et al., 2013).

Yeast two hybrid (Y2H) assays reveal both ABA-independent and ABA-dependent interactions among PYR/PYLs and PP2Cs. Y2H interactions of PYR/PYLs and PP2Cs that are dependent on exogenous ABA offer the possibility to set up screenings involving the generation of allele libraries and growth tests aimed to identify mutations that render ABA-independent interactions. Such mutations might lead in the plant cell to either receptors that interfere with PP2C function by enhancing association kinetics, steric hindrance or constitutively active receptors (not dependent on ABA-induced conformational changes) that inhibit PP2Cs in the absence of ABA. The interaction in Y2H assays of PYL4 and PP2CA, two representative members of the PYR/PYL and clade A PP2Cs families, respectively, was shown to be ABA-dependent (Lackmann et al., 2011). PYL4 shows high expression levels in different tissues and its inactivation is required to generate strongly ABA-insensitive combined pyr/pyl mutants (Gonzalez-Guzman et al., 2012). PP2CA plays a critical role to regulate both seed and vegetative responses to ABA, and regulates stomatal aperture through interaction with the anion channel SLAC1 and the kinase SnRK2.6/OST1 (Kuhn et al., 2006; Yoshida et al., 2006; Lee et al., 2009). In the absence of ABA, PP2C phosphatases interact with SnRK2 kinases to inhibit their autophosphorylation and activation. In the presence of ABA, inhibition of PP2C phosphatases by the ABA-receptor complex results in phosphorylation and activation of SnRK2 kinases, which in turn phosphorylate transcription factors that promote transcription of ABA-responsive genes.

Therefore, PP2CA is a physiologically relevant target to design PYR/PYL receptors that show a constitutive interaction with the phosphatase, affecting ABA signalling and plant stress response. Through the generation of PYL4 (At2g38310) allele libraries and Y2H assays, we identified several PYL4 mutations enabling ABA-independent interaction with PP2CA in yeast. Interestingly, upon over-expression of some PYL4 mutant receptors in Arabidopsis, we obtained enhanced sensitivity to ABA compared to wild-type PYL4 both in seed and vegetative tissues. Moreover, 35S:PYL4A194T and 5S:PYL4H82rV97A transgenic plants showed enhanced drought resistance compared to wt or 35S:PYL4 plants.

Thus, we describe a way of enhancing plant stress resistance, for example drought resistance, in a plant through the introduction of mutagenized versions of PYR/PYL, specifically PYL4, receptors. These carry single or multiple mutations and show effects in vegetative and non-vegetative tissue.

### SUMMARY OF THE INVENTION

The inventors have shown that specific modifications in a PYL/PYR polypeptide can change the properties of the protein leading to improvements of agronomically important plant traits. In particular, the inventors have shown that certain modifications enable ABA independent interaction of the mutant protein with PP2C and lead to enhanced inhibition of PP2C compared to wt receptors. The ABA-independent interaction did not lead to major inhibition of the PP2C in the absence of ABA; however it improved ABA-dependent inhibition of the PP2C, for instance at low ABA levels. In other words, the modifications result in constitutive interaction of the receptor protein with PP2C, generating additional contact points between receptor and PP2C, which leads to improved ABA-dependent inhibition of the PP2C. The inventors have also shown that when the mutant protein is expressed in transgenic plants, Arabidopsis and barley, the plants show improved stress resistance, in particular to drought stress even in the absence of a stress inducible promoter. The invention therefore relates to PYL and PYR mutant polypeptides comprising one or more amino acid mutations or modifications, for example substitutions, compared to the wild type sequence and which confer ABA-independent interaction of the PYL/PYR receptor with PP2C and enhance ABA-dependent inhibition of the PP2C as well as their use in methods for conferring stress resistance to a plant. Mutations are exemplified herein with reference to the AtPYL4 wild type polypeptide (SEQ ID NO:3). However, mutant homolog/orthologs of AtPYL4 are also within the scope of the various aspects of the invention and these have the mutations as defined herein at corresponding/equivalent positions with reference to SEQ ID NO:3.

Specifically, the invention relates to an isolated mutant nucleic acid or a nucleic acid construct comprising a mutant nucleic acid wherein said nucleic acid encodes a mutant PYL or PYR polypeptide comprising an amino acid substitution corresponding to
a) one or more of position A194, V97, C176 and/or F130 in PYL4 as set forth in SEQ ID NO:3 or
b) positions H82 and V97 in PYL4 as set forth in SEQ ID NO:3.

Thus, said mutant nucleic acid comprises SEQ ID NO:1, 2 or 4 but has one or more modifications of said sequence resulting in said nucleic acid encoding a mutant PYL or PYR polypeptide comprising an amino acid substitution. Examples of polypeptides according to the invention with specific mutations according to the invention are shown in SEQ ID NOs: 60-65.

In another aspect, the invention relates to a vector comprising an isolated mutant nucleic acid or a nucleic acid construct comprising a mutant nucleic acid wherein said nucleic acid encodes a mutant PYL or PYR polypeptide comprising an amino acid modification, preferably a substitution, at/corresponding to
a) one or more of position A194, V97, C176 and/or F130 in PYL4 as set forth in SEQ ID NO:3 or
b) positions H82 and V97 in PYL4 as set forth in SEQ ID NO:3.

In another aspect, the invention relates to a host cell comprising a vector comprising an isolated nucleic acid encoding a mutant PYL or PYR polypeptide comprising an amino acid substitution corresponding to
a) one or more of position A194, V97, C176 and/or F130 in PYL4 as set forth in SEQ ID NO:3 or
b) positions H82 and V97 in PYL4 as set forth in SEQ ID NO:3.

In a further aspect, the invention relates to a transgenic plant expressing an isolated nucleic acid encoding a mutant PYL or PYR polypeptide comprising an amino acid substitution corresponding to
a) one or more of position A194, V97, C176 and/or F130 in PYL4 as set forth in SEQ ID No. 2 or
b) positions H82 and V97 in PYL4 as set forth in SEQ ID NO:3.
   or comprising a vector comprising said isolated nucleic acid.

In yet a further aspect, the invention relates to a method for increasing stress resistance in a transgenic plant comprising, introducing and expressing a nucleic acid encoding a mutant PYL or PYR polypeptide comprising an amino acid substitutions corresponding to
a) one or more of position A194, V97, C176 and/or F130 in PYL4 as set forth in SEQ ID NO:3 or
b) positions H82 and V97 in PYL4 as set forth in SEQ ID NO:3.
or a vector comprising said nucleic acid into a plant.

In yet a further aspect, the invention relates to a method for prolonging seed dormancy/preventing early germination/inducing hyperdormancy in a transgenic plant comprising, introducing and expressing a nucleic acid encoding a mutant PYL or PYR polypeptide comprising an amino acid substitution corresponding to
a) one or more of position A194, V97, C176 and/or F130 in PYL4 as set forth in SEQ ID NO:3 or
b) positions H82 and V97 in PYL4 as set forth in SEQ ID NO:3.
or a vector comprising said nucleic acid into a plant.

In another aspect, the invention relates to a method for constitutive activation of the ABA signalling pathway comprising, introducing and expressing a nucleic acid encoding a mutant PYL or PYR polypeptide comprising an amino acid substitution corresponding to
a) one or more of position A194, V97, C176 and/or F130 in PYL4 as set forth in SEQ ID NO:3 or
b) positions H82 and V97 in PYL4 as set forth in SEQ ID NO:3.
or a vector comprising said nucleic acid into a plant.

The invention also relates to a method for inhibiting the activity of a PP2C in a transgenic plant comprising, introducing and expressing a nucleic acid encoding a mutant PYL or PYR polypeptide comprising an amino acid substitution corresponding to
a) one or more of position A194, V97, C176 and/or F130 in PYL4 as set forth in SEQ ID NO:3 or
b) positions H82 and V97 in PYL4 as set forth in SEQ ID NO:3.
or a vector comprising said nucleic acid into a plant.

The invention relates to a method for producing a transgenic plant with increased stress resistance comprising introducing and expressing a nucleic acid encoding a mutant PYL or PYR polypeptide comprising an amino acid substitution corresponding to
a) one or more of position A194, V97, C176 and/or F130 in PYL4 as set forth in SEQ ID NO:3 or
b) positions H82 and V97 in PYL4 as set forth in SEQ ID NO:3.
or a vector comprising said nucleic acid into a plant.

The invention relates to a plant obtained or obtainable by a method of the invention ro described herein. Such methods include methods for malign transgenic plants as well as methods using targeted gene editing or mutagenesis.

In a final aspect, the invention relates to the use of a nucleic acid encoding a mutant PYL or PYR polypeptide comprising an amino acid substitution corresponding to
a) one or more of position A194, V97, C176 and/or F130 in PYL4 as set forth in SEQ ID NO:3 or
b) positions H82 and V97 in PYL4 as set forth in SEQ ID NO:3.
or a vector comprising said nucleic acid for increasing stress resistance for prolonging seed dormancy or activating the ABA signalling pathway in a plant.

In one embodiment of the various aspects above, the mutant PYL or PYR polypeptide is AtPYL4. In another embodiment, the mutant PYL or PYR polypeptide is a homolog/ortholog or functional variant of AtPYL4.

The invention is further described in the following non-limiting figures.

### FIGURES

FIG. 1. Identification of PYL4 mutations that generate ABA-independent interaction with PP2CA in a Y2H assay. A, Interaction of PYL4 or PYL4 mutants (baits, fused to the Gal4 binding domain) and either PP2CA or HAB1 (preys, fused to the Gal4 activating domain). Interaction was determined by growth assay on medium lacking His and Ade. When indicated, the medium was supplemented with 50 μM ABA. Dilutions (10⁻¹, 10⁻², 10⁻³) of saturated cultures were spotted onto the plates and photographs were taken after 7 d. B, Alignment of PYR1, PYL1, PYL2 and PYL4 amino acid sequences and secondary structure. Location of the PYL4 mutations is marked by asterisks in the bottom line. Black boxes indicate the position of the gate and latch loops. Grey boxes indicate the position of the C-terminal α-helix. Asterisks in the top line mark K59, E94, Y120, S122 and E141 residues involved in ABA binding.

FIG. 2. Activity assays of PYL4 and PYL4 mutants. A, B, Phosphatase activity of either PP2CA (A) or HAB1 (B) was measured in vitro using p-nitrophenyl phosphate as a substrate in the absence or presence of PYL4 or different PYL4 mutant versions at the indicated ABA concentrations. Assays were performed in a 100 μl reaction volume that contained 2 μM phosphatase and 4 μM receptor. Data are averages ±SE from three independent experiments. * indicates p<0.05 (Student's t test) when comparing data of mutant and wt PYL4 in the same assay conditions. C, Effect of PYL4A194T or PYL4 on PP2CA-mediated dephosphorylation of OST1, ΔC-ABF2, ΔC-ABI5 and SLAC11-186 phosphorylated proteins. A 1:10 phosphatase:receptor stoichiometry was used in this assay. The quantification of the autoradiography (numbers below) shows the percentage of phosphorylated substrate in each experiment relative to the first reaction (100% in the absence of PP2CA).

D, PYL4^{A194T} prevents better than PYL4 the PP2CA-mediated dephosphorylation of OST1, ABF2 (1-173), ABI5 (1-200), and SLAC1 (1-186). Value 1 expresses protection of each substrate in the absence of ABA, and the normalized ratio expresses the fold number that either PYL4^{A194T} or PYL4 enhanced protection of the substrate at the indicated concentration of ABA. A 1:1 phosphatase:receptor stoichiometry was used in this assay.

FIG. 3. BiFC assay shows a different interaction of PYL4 or PYL4A194T and PP2CA in tobacco leaves. PYL4A194T binds ΔNPP2CA in the absence of ABA in vitro. A, Laser scanning confocal imaging of epidermal leaf cells infiltrated with a mixture of Agrobacterium suspensions harbouring the indicated BiFC constructs and the silencing suppressor p19. B, Quantification of the fluorescent protein signal. Images of panel A were analyzed using ImageJ software and signal intensity was calculated after subtracting the mean background density. C, SDS-PAGE showing the Ni2+ affinity chromatography purification from cell lysates containing recombinant 6His-ΔNPP2CA and either PYL4 (top) or PYL4A194T (bottom). A lane showing PYL4 and PYL4A194T is also displayed at the right of each gel. SF, FT and E1 to E4 stand for the soluble fraction, the flow through the column and the eluted fractions at 500 mM imidazole, respectively. In the absence of ABA, PYL4A194T co-purifies with 6His-ΔNPP2CA while PYL4 does not. D, Elution profiles after size exclusion chromatography in absence of ABA of pure PYL4A194T (grey curve with peak at about 0.22), 6His-ΔNPP2CA (grey curve with lowest peak) and the eluted fractions described above containing the co-purified PYL4A194T/6His-ΔNPP2CA (black) proteins. Insets from each peak show SDS-PAGE analysis. The figure shows the formation of 1:1 PYL4A194T:6His-ΔNPP2CA complex and the monomeric nature of PYL4A194T. E, SDS-PAGE shows a pull-down assay where 6His-ΔNPP2CA is incubated with PYL4 or PYL4A194T in absence or presence of 100 μM ABA.

FIG. 4. Enhanced sensitivity to ABA-mediated inhibition of seedling establishment and early seedling growth in PYL4 and PYL4^{A194T} OE lines compared to non-transformed Col plants. A, Immunoblot analysis using antibody against HA tag to quantify expression of PYL4, PYL4V97A, PYL4A194T, PYL4C176R F130Y and PYL4H82R V97A in 21-d-old seedlings of T3 transgenic lines (top). Ponceau staining is shown below as protein loading control. B, C, ABA-mediated inhibition of seedling establishment and early seedling growth in PYL4 and different PYL4^{mutant} OE lines compared to non-transformed Col plants. B, Approximately 100 seeds of each genotype (three independent experiments) were sown on MS plates lacking or supplemented with 0.25 or 0.5 μM ABA. Seedlings were scored for the presence of both green cotyledons and the first pair of true leaves after 8 d. Values are averages ±SE. C, Photographs of representative seedlings were taken 20 d after sowing. D, Quantification of ABA-mediated early seedling growth inhibition in PYL4 and different PYL4mutant OE lines compared to non-transformed Col plants. Data were obtained by measuring maximum rosette radius after 20 d and are averages ±SE from three independent experiments.

FIG. 5. Enhanced sensitivity to ABA-mediated inhibition of root growth of PYL4 and PYL4^{A194T} OE lines compared to non-transformed Col plants. A, Photograph of representative seedlings 10 d after the transfer of 4-d-old seedlings to MS plates lacking or supplemented with 10 μM ABA. B, C, Quantification of ABA-mediated root or shoot growth inhibition, respectively (values are means±SE, growth of Col wt on MS medium was taken as 100%). * indicates p<0.05 (Student's t test) when comparing data of PYL4 or PYL4^{A194T} OE plants to non-transformed Col plants in the same assay conditions. D, PYL4^{A194T} OE plants show partial constitutive up-regulation of ABA responsive genes in the absence of exogenous ABA. Expression of two ABA-inducible genes, RAB18 and RD29B, in Col, PYL4 and PYL4^{A194T} OE plants was analyzed by quantitative RT-PCR in RNA samples of 2-week-old seedlings that were either mock or 10 μM ABA-treated for 3 h. Data indicate the expression level (values are means±SE) of the RAB18 and RD29B genes in each column with respect to mock-treated Col (value 1).

FIG. 6. Leaf gas-exchange measurements reveal both reduced stomatal conductance and transpiration in PYL4^{A194T} OE plants compared to non-transformed Col and PYL4 OE plants. A, Gst and B, Transpiration values of non-transformed Col, PYL4 and PYL4^{A194T} OE plants. Plants were kept in custom-made whole-rosette gas exchange measurement device (see Kollist et al. 2007) and Gst and transpiration were followed during a diurnal dark/light cycle for 27 hr. Values are mean±SE (n=5). White and black bars above represent light and dark periods, respectively. C, Reduced stomatal aperture of both PYL4 and PYL4^{A194T} OE lines compared to non-transformed Col plants. D, Loss of fresh weight of 15-d-old plants submitted to the drying atmosphere of a laminar flow hood. E, Quantification of water loss in 4-week-old plants after 11 d without watering. Data shown are the average amounts of water loss measured in 10 leaves (μL/g fresh weight) collected from 5 different plants. Values are means±SE (n=10). * indicates p<0.05 (Student's t test) when comparing data of OE lines and non-transformed Col plants in the same assay conditions.

FIG. 7. PYL4A194T OE plants show enhanced drought and dehydration resistance. A, Enhanced drought resistance of PYL4^{A194T} OE plants with respect to non-transformed Col or PYL4 OE plants. Two-week-old plants were deprived of water for 19 days and then re-watered. Photographs were taken at the start of the experiment (0-d), after 16 and 19 days of drought (16 d, 19 d) and 2 days after re-watering (21 d from 0 d). Shoot was cut to better show the effect of drought on rosette leaves. B, Quantification of shoot-growth (maximum rosette radius) of non-transformed Col, PYL4 and PYL4^{A194T} OE plants during the course of the experiment. Measurements were taken at different times (2, 5, 7 and 9 d) after the start of the experiment and values at 0 d were taken as 100%. Values are means±SE (n=10). C, Survival percentage of non-transformed Col, PYL4 and PYL4A194T OE plants 3 d after re-watering. Values are means±SE from three independent experiments (n=10 each). D, PYL4A194T and PYL4H82RV97A OE plants show enhanced resistance to dehydration. 2-week-old plants grown on MS plates were dehydrated by opening the lid in a laminar flow hood for 12 hours (25° C.±1° C., 25%±2% relative humidity), next rehydrated and survival was scored 3 d later.

FIG. 8. Alignment of AtPYL4 protein with PYL4 orthologous proteins in crop plants.

FIG. 9. Transgenic barley plants overexpressing either PYL4A194T or PYL4H82R V97A show enhanced drought tolerance at the vegetative stage. (A, C) Four-week-old plants were watered with tap water for 12-d (−D) or were submitted to drought treatment for 12-d (+D). (B, D) Plants submitted to drought were rewatered (RW) and a photograph was taken 5-d after rewatering (+D, +RW).

FIG. 10. Transgenic barley overexpressing either PYL4A194T or PYL4H82R V97A show enhanced fresh weight t (FW) compared to nontransformed plants after drought t treatment. Four-week-old plants were watered with tap water for 12-d (−D) or were submitted to drought treatment for 12-d (+D). One leaf per plant (10 individual plants for each genetic background) was weighed (FW) and dried for 16 h at 70° C. and weighed again to obtain the dry weight (DW). Data represent average FW or DW/leaf ±SE. * indicates p<0.05 (Student's t test) when comparing data of transgenic lines to non-transformed plants in the same assay conditions.

FIG. 11. Transgenic barley plants overexpressing either PYL4A194T or PYL4H82R V97A show enhanced drought tolerance compared to nontransformed plants (A) Four-week-old plants were watered with tap water for 18-d (B) or were submitted to drought treatment for 12-d and rewatered. Ten individual plants for each genetic background were weighed (FW). Data represent average FW/plant ±SE. * indicates p<0.05 (Student's t test) when comparing data of transgenic lines to non-transformed plants in the same assay conditions.

### DETAILED DESCRIPTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The practice of the present invention will employ, unless otherwise indicated, conventional techniques of botany, microbiology, tissue culture, molecular biology, chemistry, biochemistry and recombinant DNA technology, bioinformatics which are within the skill of the art. Such techniques are explained fully in the literature. As used herein, the words “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences. In one embodiment, the isolated nucleic acid and the isolated nucleic acid used in the various methods and plants according to the invention is PYL/PYR cDNA. Examples of such sequences are given herein.

The terms “peptide”, “polypeptide” and “protein” are used interchangeably herein and refer to amino acids in a polymeric form of any length, linked together by peptide bonds.

For the purposes of the invention, “transgenic”, “transgene” or “recombinant” means with regard to, for example, a nucleic acid sequence, an expression cassette, gene construct or a vector comprising the nucleic acid sequence or an organism transformed with the nucleic acid sequences, expression cassettes or vectors according to the invention, all those constructions brought about by recombinant methods in which either

(a) the nucleic acid sequences encoding proteins useful in the methods of the invention, or
(b) genetic control sequence(s) which is operably linked with the nucleic acid sequence according to the invention, for example a promoter, or
(c) a) and b)
are not located in their natural genetic environment or have been modified by recombinant methods, it being possible for the modification to take the form of, for example, a substitution, addition, deletion, inversion or insertion of one or more nucleotide residues. The natural genetic environment is understood as meaning the natural genomic or chromosomal locus in the original plant or the presence in a genomic library. In the case of a genomic library, the natural genetic environment of the nucleic acid sequence is preferably retained, at least in part. The environment flanks the nucleic acid sequence at least on one side and has a sequence length of at least 50 bp, preferably at least 500 bp, especially preferably at least 1000 bp, most preferably at least 5000 bp. A naturally occurring expression cassette—for example the naturally occurring combination of the natural promoter of the nucleic acid sequences with the corresponding nucleic acid sequence encoding a polypeptide useful in the methods of the present invention, as defined above—becomes a transgenic expression cassette when this expression cassette is modified by non-natural, synthetic (“artificial”) methods such as, for example, mutagenic treatment. Suitable methods are described, for example, in U.S. Pat. No. 5,565,350 or WO 00/15815 both incorporated by reference.

A transgenic plant for the purposes of the invention is thus understood as meaning, as above, that the nucleic acids used in the method of the invention are not at their natural locus in the genome of said plant, it being possible for the nucleic acids to be expressed homologously or heterologously. However, as mentioned, transgenic also means that, while the nucleic acids according to the different embodiments of the invention are at their natural position in the genome of a plant, the sequence has been modified with regard to the natural sequence, and/or that the regulatory sequences of the natural sequences have been modified. Transgenic is preferably understood as meaning the expression of the nucleic acids according to the invention at an unnatural locus in the genome, i.e. homologous or, preferably, heterologous expression of the nucleic acids takes place. According to the invention, the transgene is integrated into the plant in a stable manner and preferably the plant is homozygous for the transgene.

The aspects of the invention involve recombination DNA technology, mutagenesis or genome editing and exclude embodiments that are solely based on generating plants by traditional breeding methods.

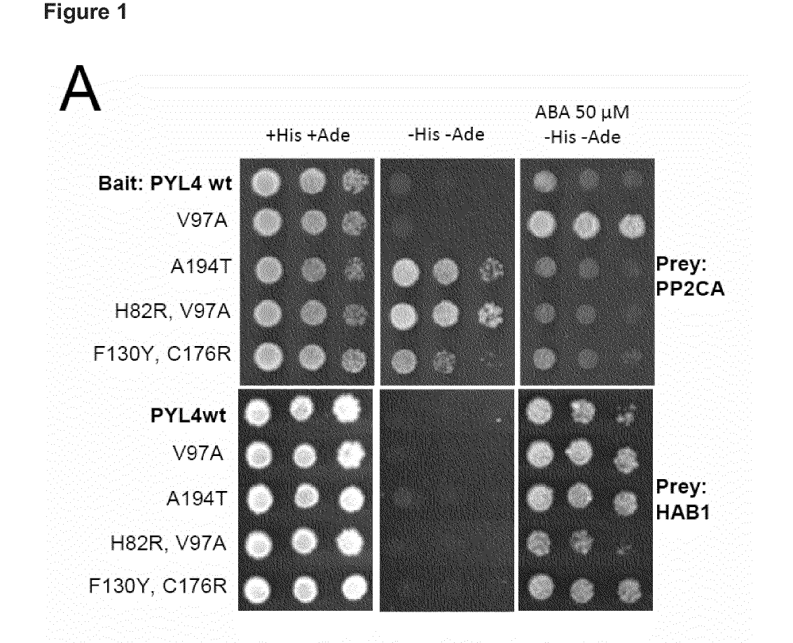

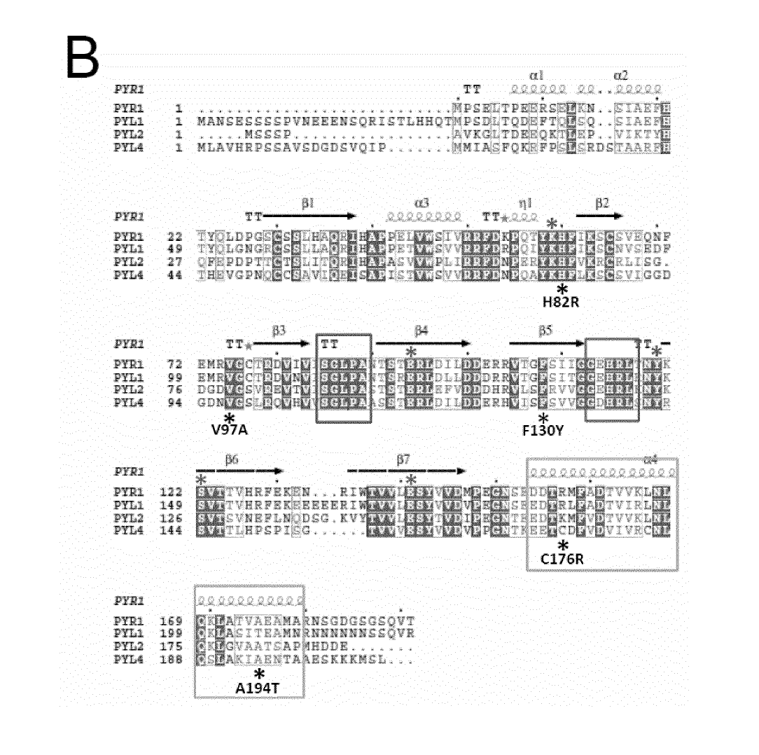

The invention relates to isolated plant PYL and PYR mutant nucleic acid and isolated mutant plant PYL and PYR polypeptides encoded by said mutant nucleic acid wherein said mutant polypeptides comprise one or more amino acid mutations or modifications, for example substitutions or deletions, compared to the wild type sequence, which promote ABA-independent interaction with PP2C and enhanced ABA-dependent inhibition of the PP2C, for instance at low ABA levels As explained herein, certain nucleic acids are modified in the mutant nucleic acids so that the resulting mutant protein is different from the wild type protein. The sequences shown herein show the wild type sequences and mutations in the mutant proteins with reference to positions in these sequences are set out herein. The invention also relates to methods for making transgenic plants with improved traits expressing said mutant polypeptides. Preferably, these mutations are located in the protein domain that interacts with a PP2C, for example PP2CA, and/or the domain that interacts with ABA (residues K59, E94, Y120, S122 and E141 in AtPYL4 as shown in FIG. 1B and with reference to the AtPYL4 protein sequence SEQ ID NO:3). Such mutations are referred to as activating mutations/substitutions herein. Also, any mutations that affect ABA signalling, for example mutations that activate (promote ABA-independent interaction with PP2C) the PYL/PYR receptor in the absence of ABA and enhance ABA-dependent inhibition of PP2C are referred to as activating mutations/substitutions herein. Thus, the mutant polypeptide/proteins according to the invention are non-naturally occurring peptides which can be generated by site-directed mutagenesis and introduced stably into plants and expressed in said plants to produce stable transgenic plants with improved traits. Said plants are preferably homozygous for the transgene. The mutations/modifications are substitutions or deletions wherein said deletions do not introduce a stop codon. Preferably, the modifications are substitutions.

In one embodiment, the polypeptide of the various aspects of the invention has one or more mutation at one or more of the positions defined herein, but does not have mutations at one or more of the following positions with reference to SEQ ID NO: 3: H82, V105, V106, L109, A111, D177, F178, V181, C185 and/or S189. Also excluded are specific activating mutations as disclosed in WO 2013/006263, including mutations in PYL/PYR polypeptides corresponding to the following positions in PYR1: V83, 184, L87, A89, M158, F159, T162, L166, K170 (incorporated by reference). In one embodiment of the various aspects of the invention, the polypeptide of the invention does not have any additional mutations other than one or more of those mutations described herein. In one embodiment, the polypeptide of the invention does not have any additional activating mutations, that is mutations that affect ABA signalling.

Thus, in a first aspect, the invention relates to an isolated mutant nucleic acid encoding a mutant PYL or PYR polypeptide comprising one or more amino acid substitutions corresponding to one or more of position A194, V97, F130 and/or C176 in PLY4 as set forth in SEQ ID NO: 3 and shown in FIG. 1B or a position corresponding thereto.

Thus, in one embodiment, the substitution is at A194 with reference to AtPLY4 as set forth in SEQ ID NO: 3 (see also SEQ ID NO: 55 and 60) or a position corresponding thereto. In another embodiment, the substitution is at V97 with reference to AtPLY4 as set forth in SEQ ID NO: 3 (see also SEQ ID NO:61) or a position corresponding thereto. In another embodiment, the substitution is at F130 with reference to AtPLY4 as set forth in SEQ ID NO: 3 (see also SEQ ID NO:64) or a position corresponding thereto. In another embodiment, the substitution is at C176 with reference to AtPLY4 as set forth in SEQ ID NO: 3 (see also SEQ ID NO:65) or a position corresponding thereto. In another embodiment, one or more of the residues at positions corresponding to A194, V97, F130 and/or C176 in PLY4 as set forth in SEQ ID NO: 3 is deleted.

The invention also relates to an isolated nucleic acid encoding a mutant PYL or PYR polypeptide comprising two amino acid substitutions corresponding to/equivalent to positions H82 and V97 as set forth in SEQ ID NO:3 and shown in FIG. 1B (see also SEQ ID NO: 56 and 62). Preferably, the isolated nucleic acid encoding a mutant PYL or PYR polypeptide has the substitutions at positions H82 and V97 or at positions corresponding thereto, but has no other activating amino acid substitutions compared to the wild type sequence. In another aspect, the amino acid substitutions at position H82 and V97 may be combined with an amino acid substitution at A194 and/or V97 and/or F130 and/or 0176 and/or mutations in other residues in the domains that interact with ABA or a PP2C.

Thus, the polypeptide preferably comprises one, two, three, four or more mutations as described above. Any combination of the substitutions or deletions specifically set out above is within the scope of the invention. For example, an amino acid substitution at position A194 or a position corresponding thereto may be combined with an amino acid substitution at V97 and/or F130 and/or 0176 or a position corresponding thereto. In one embodiment, other combinations of the mutations at positions A194, V97 and/or F130 and/or 0176 are also possible, for example with H82 and/or mutations in other residues in the domains that interact with ABA or a PP2C.

In one embodiment, an amino acid substitution at position V97 or a position corresponding thereto may be combined with an amino acid substitution at F130, A194 and/or 0176 or a position corresponding thereto. In one embodiment, an amino acid substitution at position F130 may be combined with an amino acid substitution at A194, V97 and/or 0176 or a position corresponding thereto. In one embodiment, an amino acid substitution at position 0176 may be combined with an amino acid substitution at A194, V97 and/or F130 or a position corresponding thereto.

A PP2C may be selected from HAB1 (Homology to AB11), ABI1 (Absciscic acid insensitive 1), ABI2 (Absciscic acid insensitive 2) or PP2CA. Preferably, the PP2C is PP2CA.

The amino acid substitutions at the positions set out above are preferably conservative amino acid substitutions. Conservative substitution tables are well known in the art. Alternatively, insertions can be made to render the site non-functional.

The mutations in the PYL/PYR polypeptides described herein are shown with reference to the amino acid positions as shown in SEQ NO:3 which designates the AtPLY4 wild type polypeptide sequence encoded by a nucleic acid shown in SEQ ID NOs:1, 2 or 4. Thus, in one embodiment, the mutant PYL or PYR polypeptide is encoded by a nucleic acid comprising, consisting or substantially consisting of a sequence substantially identical to SEQ ID NOs: 1, 2 or 4, a functional variant, ortholog or homolog thereof, but which has modifications so that transcription of the mutant nucleic acid results in a mutant protein comprising one or more of the mutations at the positions listed above. In other words, the mutant PYL or PYR polypeptide is encoded by a nucleic acid comprising, consisting or substantially consisting of a sequence substantially identical to SEQ ID NOs: 1, 2 or 4, a functional variant, ortholog or homolog thereof, but which comprises modifications in one or more the codons encoding the one or more residues listed above. These codons are 82, 97, 130, 176 and/or 194 in AtPYL4. As explained below, other PYL/PYR polypeptides share homology with AtPYL4 and residues for targeted manipulation that correspond to one or more of positions A194, V97, F130, H82 and 0176 in AtPYL4 can be identified by sequence comparison and alignment as described herein.

A PYL/PYR nucleic acid as used herein and according to the various aspects of the invention comprises SEQ ID NOs: 1, 2 or 4 or a functional variant or homolog/ortholog thereof, but wherein said nucleic acid is not the wild type nucleic acid shown in these sequences, but is a mutant nucleic acid that has a mutation in one or more codon which results in one or more mutation in the encoded polypeptide. Said mutation in the polypeptide is an amino acid substitution corresponding to
a) one or more of position A194, V97, C176 and/or F130 in PYL4 as set forth in SEQ ID NO:3 or
b) positions H82 and V97 in PYL4 as set forth in SEQ ID NO:3.

The term functional variant or homolog/ortholog of SEQ ID NOs: 1, 2 or 4 is described below and specific examples of such nucleic acids are also given below.

The sequence below shows the open reading frame of AtPYL4 (SEQ ID NO:2)

ATGCTTGCCGTTCACCGTCCTTCTTCCGCCGTATCAGACGGAGATTCCGTTCAGATTCCGATGATGATCG
CGTCGTTTCAAAAACGTTTTCCTTCTCTCTCACGCGACTCCACGGCCGCTCGTTTTCACACACACGAGGT
TGGTCCTAATCAGTGTTGCTCCGCCGTTATTCAAGAGATCTCCGCTCCAATCTCCACCGTTTGGTCCGTC
GTACGCCGCTTTGATAACCCACAAGCTTACAAACACTTTCTCAAAAGCTGTAGCGTCATCGGCGGAGACG
GCGATAACGTTGGTAGCCTCCGTCAAGTCCACGTCGTCTCTGGTCTCCCCGCCGCTAGCTCCACCGAGAG
ACTCGATATCCTCGACGACGAACGCCACGTCATCAGCTTCAGCGTTGTTGGTGGTGACCACCGGCTCTCT
AACTACCGATCCGTAACGACCCTTCACCCTTCTCCGATCTCCGGGACCGTCGTTGTCGAGTCTTACGTCG
TTGATGTTCCTCCAGGCAACACAAAGGAAGAGACTTGTGACTTCGTTGACGTTATCGTACGATGCAATCT
TCAATCTCTTGCGAAAATAGCCGAGAATACTGCGGCTGAGAGCAAGAAGAAGATGTCTCTGTGA

The one or more codons which are mutated according to the various aspects of the invention to obtain the mutations in the polypeptides of the invention are in bold and underlined. This is also shown below.

| | Amino | Examples of |
|---|---|---|
| Codon | acid residue | modified codons |
| GCC | A194 | ACT |
| GTT | V97 | GCT |
| TGT | C176 | CGT |
| TTC | F130 | TAC |
| CAC | H82 | CGC |

Examples of polypeptides according to the invention with specific mutations according to the invention are shown in SEQ ID NOs: 60-65. Functional variants or homolog/orthologs thereof with mutations at positions corresponding to the mutated positions in AtPYL4 are also within the scope of the invention.

In one embodiment, the mutant polypeptide according to the invention comprises an amino acid substitution corresponding to position A194 with reference to SEQ ID NO:3. Thus, the invention relates to an isolated mutant nucleic acid encoding a mutant PYL or PYR polypeptide comprising an amino acid substitution corresponding to position A194 with reference to SEQ ID NO:3 (see SEQ ID NO. 55 and 60). A194 is located in the C-terminal part of the C-terminal helix. The inventors have demonstrated that this mutation alone without the presence of additional mutations can confer a drought resistant phenotype to plants compared to a control wild type plant in the absence of other activating mutations in the domains that interact with ABA or a PP2C. The effect is observed in seeds and in vegetative tissue. In one embodiment, the mutant polypeptide does therefore not comprise any additional activating mutations. The A residue at position 194 or a position corresponding thereto in an AtPYL4 homolog/ortholog may be substituted with T, V, L, M, I or S. In a preferred embodiment, the substitution is with T, for example A194T in SEQ ID NO:3. In another embodiment, the mutant polypeptide according to the various aspects of the invention does not comprise any additional mutations.

Thus, in one embodiment, the polypeptide has an amino acid substitution selected from A194T and no other activating mutations in other residues in the domains that interact with ABA or a PP2C are present. In one embodiment, no other mutations are present. In one embodiment, the polypeptide does not have mutations at one or more of the following positions with reference to SEQ ID NO:3: H82, V105, V106, L109, A111, D177, F178, V181, C185 and/or S189. Also excluded are specific activating mutations as disclosed in WO 2013/006263 (incorporated by reference). In one embodiment, the mutant polypeptide with a mutation at A194 with reference to SEQ ID NO:3 is PYL4, a functional variant, homolog or ortholog thereof as described herein.

In another embodiment of the various aspects of the invention, the mutant polypeptide comprises an amino acid substitution corresponding to position V97 with reference to SEQ ID NO:3. In one embodiment, the polypeptide does not comprise any additional activating mutations. In another embodiment, the polypeptide does not comprise any additional mutations. In one embodiment, the polypeptide does not have mutations at the following positions with reference to SEQ ID NO:3: H82, V105, V106, L109, A111, D177, F178, V181, C185 and/or S189 or at a position corresponding thereto. Also excluded are specific activating mutations as disclosed in WO 2013/006263. In one embodiment, the mutant polypeptide is PYL4, a functional variant, homolog or ortholog thereof as described herein.

According to the various embodiment, the V residue at position 97 or a position corresponding thereto may be substituted with L, M, I, S or T. In a preferred embodiment, the substitution is 97A. In one embodiment, the mutant polypeptide is PYL4, a functional variant, homolog or ortholog thereof as described herein.

In another embodiment, the mutant polypeptide comprises an amino acid substitution corresponding to positions F130 and/or 0176 with reference to SEQ ID NO:3 or a position corresponding thereto. In one embodiment, the polypeptide does not comprise any further activating additional mutations. In another embodiment, the polypeptide does not comprise any additional mutations. The F residue at position 130 may be substituted with W. The C residue at position 176 may be substituted with K or H.

In one embodiment, the polypeptide does not have mutations at the following positions with reference to SEQ ID NO:3: H82, V105, V106, L109, A111, D177, F178, V181, C185 and/or S189 or at a position corresponding thereto. Also excluded are specific activating mutations as disclosed in WO 2013/006263. In a preferred embodiment, the substitution is F130Y and/or C176R. In one embodiment, the mutant polypeptide is PYL4, a functional variant, homolog or ortholog thereof as described herein. In one embodiment, no other mutations are present.

In another embodiment, the mutant polypeptide comprises an amino acid substitution corresponding to position H82 and an amino acid substitution corresponding to position V97 with reference to SEQ ID NO:3. Thus, at least two mutations are present in the polypeptide. Preferably, the polypeptide does not comprise any additional activating mutations. In another embodiment, the polypeptide does not comprise any additional mutations. In one embodiment, the polypeptide does not have mutations at the following positions with reference to SEQ ID NO:3: V105, V106, L109, A111, D177, F178, V181, C185 and/or S189 or at a position corresponding thereto. Also excluded are specific activating mutations as disclosed in WO 2013/006263. The H residue at position 82 may be substituted with K, N, Q, F, Y, W or P. In one embodiment, the residue is not P. In a preferred embodiment, the substitution is H82R. The V residue at position 97 may be substituted with L, M, I, S, T In one embodiment, the mutant polypeptide is PYL4, a functional variant, homolog or ortholog thereof as described herein.

The term “functional variant of a nucleic acid or peptide sequence” as used herein with reference to a mutant of SEQ ID NOs: 1, 2, 3 or 4 or homologs thereof as described herein refers to a variant gene sequence or part of the gene sequence which retains the biological function of the full mutant sequence, for example confers increased stress resistance/yield and ABA-independent interaction with a PP2C when expressed in a transgenic plant. A functional variant also comprises a variant of the gene of interest which has sequence alterations that do not affect function, for example in non-conserved residues. Also encompassed is a variant that is substantially identical, i.e. has only some sequence variations, for example in non-conserved residues, to the wild type sequences but which includes the target mutations as shown herein and is biologically active. Variants have at least 80%, 81%, 82%, 83%, 84%, 85%, 86%, 87%, 88%, 89%, 90%, 91%, 92%, 93%, 94%, 95%, 96%, 97%, 98%, or 99% overall sequence identity to the wild type sequence. A variant may for example have restriction sites introduced in the coding sequence to facilitate cloning (see examples).

Thus, it is understood, as those skilled in the art will appreciate, that the aspects of the invention, including the methods and uses, encompass not only a mutant nucleic acid sequence comprising, consisting essentially or consisting of SEQ ID NOs: 1, 2 or 4 or a mutant polypeptide comprising, consisting essentially or consisting or SEQ ID NO: 3, which have the mutations described herein but are otherwise shown as in the referenced sequences, but also functional variants of the mutant sequences of SEQ ID NO: 1 to 4 or homologs thereof that do not affect the biological activity and function of the resulting mutant protein. In other words, the additional variations present in the variants do not affect ABA interaction or other biological functions and the phenotype of the transgenic plant expressing the variant is that of the transgenic plant expressing the mutant peptide as described above. Alterations in a nucleic acid sequence which result in the production of a different amino acid at a given site that do however not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products. Alterations of the variants are not activating mutations.

Also, the various aspects of the invention the aspects of the invention, including the methods and uses, encompass not only a PYL, but also a fragment thereof. By “fragment” is intended a portion of the nucleotide sequence or a portion of the amino acid sequence and hence of the protein encoded thereby. Fragments of a nucleotide sequence encode protein fragments that retain the biological activity of the native protein and hence act to modulate responses to ABA.

In one embodiment according to the various aspects of the invention, the PYL/PYR mutant polypeptide is a mutant PYL4 polypeptide of AtPYL4 as shown in SEQ ID NO:3. The mutant has a modification, preferably a substitution at one or more of position A194, V97, C176 and/or F130 in PYL4 as set forth in SEQ ID NO:3 or at positions H82 and V97 in PYL4 as set forth in SEQ ID NO:3. Examples are shown in SEQ ID NOs: 60-65.

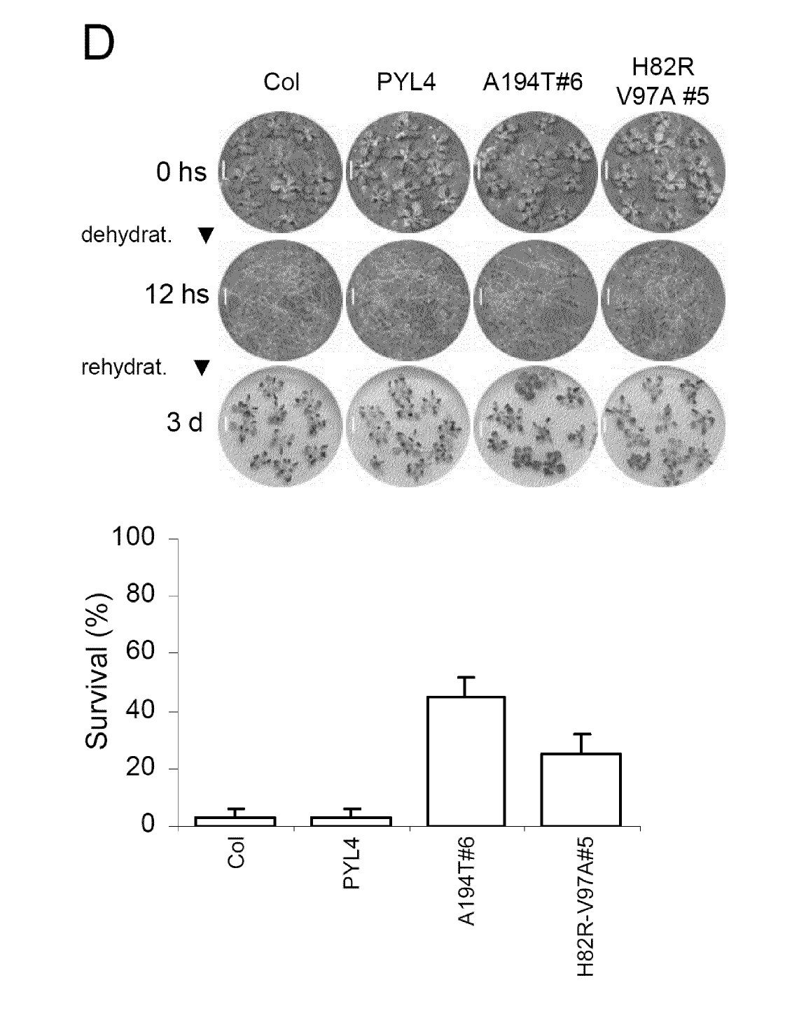

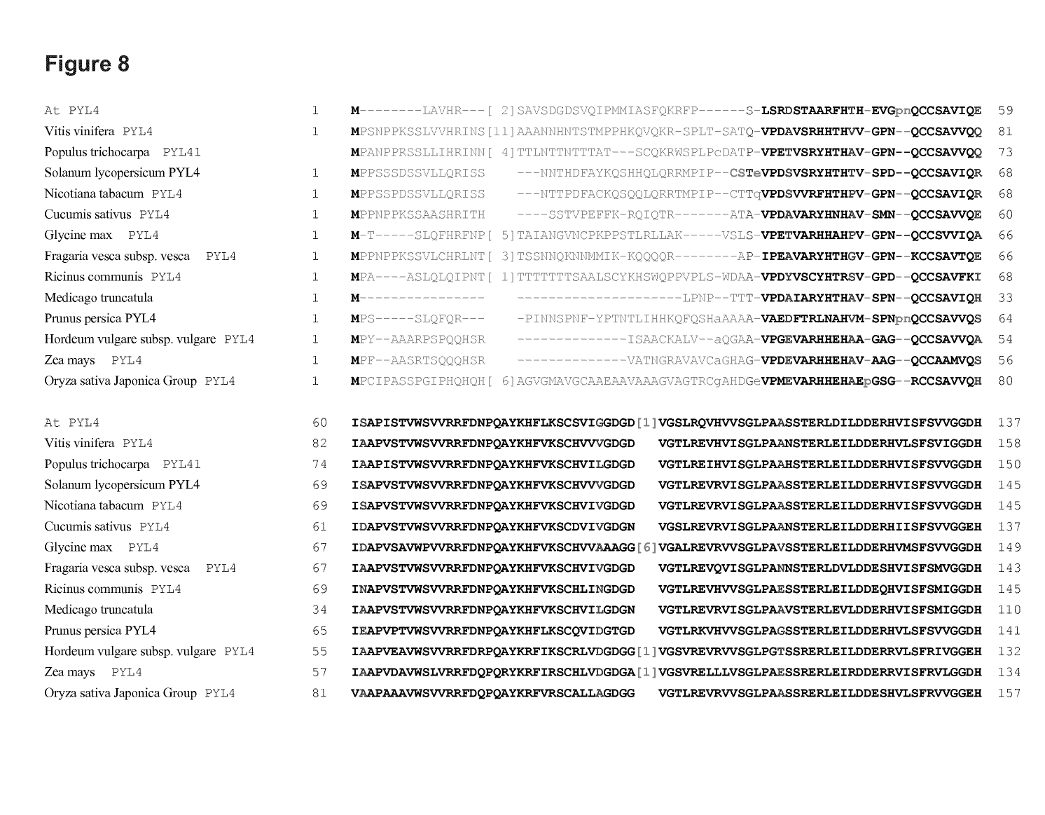

However, the invention also extends to functional homologs/orthologs of AtPYL4 with mutations in corresponding/equivalent positions when compared to the AtPYL4 sequence. A functional variant or homolog of AtPYL4 as shown in SEQ ID NO:3 is a PYL4 peptide which is biologically active in the same way as SEQ ID NO:3, in other words, for example it confers increased stress resistance, preferably against drought. The term functional homolog includes AtPYL4 orthologs in other plant species. In a preferred embodiment of the various aspects of the invention, the invention relates specifically to AtPYL4 or orthologs of AtPYL4 in other plants. Non-limiting examples of these are shown in FIG. 8 and corresponding sequences for nucleic acids and proteins are shown as SEQ ID NOs: 5-42. In one embodiment, the AtPYL4 protein homolog/ortholog is as shown in SEQ ID NOs: 7, 9, 12, 14, 16, 19, 21, 23, 26, 28, 31, 34, 37, 39 or 42 but wherein said protein has one or more mutation at one or more position corresponding to positions set out herein with reference to SEQ ID NO:3, that is positions corresponding to the target residues in the AtPYL4 as set out herein. The are selected from

a) one or more of position A194, V97, C176 and/or F130 in PYL4 as set forth in SEQ ID NO:3 or
b) positions H82 and V97 in PYL4 as set forth in SEQ ID NO:3.

Corresponding wild type nucleic acid sequences are also shown herein as SEQ ID NOs: 6, 8, 10, 11, 13, 15, 17, 18, 20, 22, 24, 25, 27, 29, 30, 32, 33, 37, 38, 40 and 41. These have mutations in codons equivalent to the mutated codons in AtPYL4 as explained herein. Variants of homologous protein/nucleic acid sequences that retain the biological activity of the mutant sequence and which have at least 80%, 81%, 82%, 83%, 84%, 85%, 86%, 87%, 88%, 89%, 90%, 91%, 92%, 93%, 94%, 95%, 96%, 97%, 98%, or 99% overall sequence identity to the sequence listed above are also included. This list is non-limiting and other homologous sequences of plants that are described herein, for example the AtPYL4 homolog/ortholog according to the various aspects of the invention is from other preferred plants, such as from crop plants. In a preferred embodiment, the AtPYL4 homolog/ortholog is from maize, rice, wheat, oilseed rape, sorghum, soybean, potato, tomato, grape, barley, pea, bean, field bean, lettuce, cotton, sugar cane, sugar beet, broccoli or other vegetable brassicas or poplar are also included within the scope of the invention. In one embodiment, the AtPYL4 protein homolog/ortholog is as shown in SEQ ID NOs: 43-54.

Thus, the invention also relates to isolated mutant nucleic acids encoding functional homologs/orthologs of the AtPYL4 polypeptide with one or more mutation in corresponding positions when compared to the AtPYL4 mutant sequence of the invention and to isolated functional homologs/orthologs of the AtPYL4 mutant polypeptide with one or more mutation in corresponding positions when compared to the AtPYL4 sequence. It also extends to transgenic plants that express functional homologs/orthologs of the AtPYL4 polypeptide with one or more mutation at a position corresponding to A194, V97, F130, H82 and/or C176 or H82 and V97 when compared to the AtPYL4 sequence.

The homologue of a AtPYL4 polypeptide according to the various aspects of the invention has, in increasing order of preference, at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the wild type amino acid represented by SEQ ID NO: 3. Preferably, overall sequence identity is 85%, 86%, 87%, 88%, 89%, 90%, 91%, 92%, 93%, 94%, 95%, 96%, 97%, 98%, or 99%. In another embodiment, the homolog of a AtPYL4 nucleic acid sequence has, in increasing order of preference, at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the nucleic acid represented by SEQ ID NOs: 1, 2 or 4. Preferably, overall sequence identity is 90%, 91%, 92%, 93%, 94%, 95%, 96%, 97%, 98%, or 99%. The overall sequence identity is determined using a global alignment algorithm known in the art, such as the Needleman Wunsch algorithm in the program GAP (GCG Wisconsin Package, Accelrys).

Preferably, the AtPYL4 homolog/ortholog according to the various aspects of the invention comprises a conserved gate motif (residues 107-111 in AtPYL4: SGLPA; SEQ ID NO:57) and/or latch loops (residues 135-139 in AtPYL4: GDHRL; SEQ ID NO:58) and/or a conserved C-terminal alpha helix (residues 173-198 in AtPYL4: EETCDFVDVIVRCNLQSLAKIAENTA; SEQ ID NO:59) as shown in FIG. 1B. In one embodiment, the AtPYL4 homolog/ortholog has a domain with at least 99% homology to SGLPA and/or a domain with at least 99% homology to GDHRL and/or a domain with at least 95%, 96%, 97% or 99% homology to EETCDFVDVIVRCNLQSLAKIAENTA (SEQ ID NO:59). In a preferred embodiment, all domains identical to or with homology as defined above to all three domains are present. Thus, term AtPYL4 homolog/ortholog refers to a protein characterized at least in part by the presence of one or more or all of these domains.

Suitable homologues or orthologues can be identified by sequence comparisons and identifications of conserved domains using databases such as NCBI and Paint ensemble and alignment programmes known to the skilled person. The function of the homologue or orthologue can be identified as described herein and a skilled person will thus be able to confirm the function when expressed in a plant. Thus, one of skill in the art will recognize that analogous amino acid substitutions listed above with reference to SEQ ID NO:3 can be made in PYL4 receptors from other plants by aligning the PYL4 receptor polypeptide sequence to be mutated with the AtPYL4 receptor polypeptide sequence as set forth in SEQ ID NO: 3.

Thus, the nucleotide sequences of the invention and described herein can be used to isolate corresponding sequences from other organisms, particularly other plants, more particularly cereals. In this manner, methods such as PCR, hybridization, and the like can be used to identify such sequences based on their sequence homology to the sequences described herein. Sequences may be isolated based on their sequence identity to the entire sequence or to fragments thereof. In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen plant. The hybridization probes may be genomic DNA fragments, cDNA fragments, RNA fragments, or other oligonucleotides, and may be labelled with a detectable group, or any other detectable marker. Thus, for example, probes for hybridization can be made by labeling synthetic oligonucleotides based on the ABA-associated sequences of the invention. Methods for preparation of probes for hybridization and for construction of cDNA and genomic libraries are generally known in the art and are disclosed in Sambrook, et al., (1989) Molecular Cloning: A Library Manual (2 d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Duration of hybridization is generally less than about 24 hours, usually about 4 to 12. Stringent conditions may also be achieved with the addition of destabilizing agents such as formamide.

As a non-limiting example, an amino acid substitution in PYL4 that is analogous/equivalent to the amino acid substitution A194 in AtPYL4 as set forth in SEQ ID NO: 3 can be determined by aligning the amino acid sequences of AtPYL4 (SEQ ID NO:3) and a PYL4 amino acid sequence from another plant species and identifying the position corresponding to A194 in the PYL4 from another plant species as aligning with amino acid position A194 of AtPYL4. This is shown in FIG. 8. The other amino acid substitution in PYL4 as described herein can be determined in the same way.

For example, according to the various aspects of the invention, a nucleic acid encoding a mutant PYL/PYR polypeptide, for example PYL4 which is a mutant version of the endogenous wt a mutant PYL/PYR polypeptide, for example PYL4 peptide in a plant may be expressed in said plant by recombinant methods. In another embodiment, a mutant a mutant PYL/PYR polypeptide, for example PYL4, which is a mutant version of a mutant PYL/PYR polypeptide, for example PYL4 peptide in a plant may be expressed in any plant of a second species as defined herein by recombinant methods.

For example, a mutant AtPYL4 or a homolog thereof according to the invention may be expressed in a crop plant. For example, a mutant AtPYL4 may be expressed in barley.

In one particular embodiment of the various aspects of the invention, the mutant nucleic acid is substantially identical to AtPYL4 as shown in SEQ ID No. 1, 2 or 4, a functional variant, homolog or otholog thereof, but has one or more modification of a codon as described herein so that it encodes a mutant polypeptide comprising an amino acid substitution corresponding to position A194 with reference to SEQ ID NO:3 (see also SEQ ID NO:55 and 60) or a position corresponding thereto.

In another embodiment, the various aspects of the invention relate to another member of the PYL/PYR receptor family wherein said PYL/PYR polypeptide is a mutant polypeptide comprising one or more amino acid modifications, selected from one or more amino acid substitutions corresponding to one or more of position A194, V97, F130 and/or C176 in PYL4 as set forth in SEQ ID NO:3 or amino acid substitutions corresponding to H82 and V97 in PLY4 as set forth in SEQ ID NO:3 or at a position corresponding thereto. In one embodiment, the mutant polypeptide comprises an amino acid substitutions corresponding to one or more of position A194, for example A194T. This may be present without the presence of other modifications or may be combined with other mutations in the amino acid sequence.

PYL/PYR bind PP2C via a PP2C binding interface which is characterised by conserved residues, including H82 in AtPYL4. A common motif of the PYL/PYR receptor family is also the conserved C-terminal helix which includes A194 in AtPYL4. A nucleic acid encoding a PYR/PYL polypeptide or a PYR/PYL polypeptide has, in increasing order of preference, at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the nucleic acid represented by SEQ ID NO: 1, 2 or 4 or to the polypeptide represented by SEQ ID NO: 3 or a homolog thereof. Preferably, overall sequence identity is 90%, 91%, 92%, 93%, 94%, 95%, 96%, 97%, 98%, or 99%.

Thus, term “PYR/PYL receptor polypeptide” refers to a protein characterized in part by the presence of one or more or all of a polyketide cyclase domain 2 (PF 10604), a polyketide cyclase domain 1 (PF03364), and a Bet V I domain (PF03364), which in wild-type form mediates abscisic acid (ABA) and ABA analog signaling. A wide variety of PYR/PYL receptor polypeptide sequences are known in the art. In some embodiments, a PYR/PYL receptor polypeptide comprises a polypeptide that is substantially identical to AtPYL4 (SEQ ID NO:3), AtPYL1 (SEQ ID NO:43), AtPYL2 (SEQ ID NO:44), AtPYL3 (SEQ ID NO:45), AtPYL5 (SEQ ID NO:46), AtPYL6 (SEQ ID NO:347), AtPYL7 (SEQ ID NO:48), AtPYL8 (SEQ ID NO:49), AtPYL9 (SEQ ID NO: 50), AtPYL10 (SEQ ID NO: 51), AtPYL11 (SEQ ID NO: 52), AtPYL12 (SEQ ID NO: 53), or AtPYL13 (SEQ ID NO: 4) or homologs thereof, but has one or more mutation at the positions corresponding to the targets in AtPYL4 as set out herein. In some embodiments, a PYR/PYL receptor polypeptide comprises a PYR polypeptide. In some embodiments, the PYR/PYL receptor polypeptide is as shown in FIG. 1B with corresponding mutations.

Orthologs of PYR/PYL receptor polypeptides in other plant species, for example as shown in FIG. 8 and in SEQ ID No. 43-54, are also within the various aspects of the invention.

In another aspect, the invention relates to a nucleic acid construct or vector comprising an isolated nucleic acid as described herein. Thus, the vector comprises an isolated nucleic acid encoding a mutant PYL/PYR polypeptide, for example PYL4, comprising one or more amino acid substitutions corresponding to one or more of position A194, H82, V97, F130 or C176 in PLY4 as set forth in SEQ ID NO:3 or comprising amino acid substitutions corresponding to positions H82, and V97 as set forth in SEQ ID NO:3. For example, the substitution may be at position A194, such as A194T. As explained above, in one embodiment, other activating mutations are not present. Preferably, the vector further comprises a regulatory sequence which directs expression of the nucleic acid. In one embodiment, no other mutations are present.

The terms “regulatory element”, “regulatory sequence”, “control sequence” and “promoter” are all used interchangeably herein and are to be taken in a broad context to refer to regulatory nucleic acid sequences capable of effecting expression of the sequences to which they are ligated. The term “promoter” typically refers to a nucleic acid control sequence located upstream from the transcriptional start of a gene and which is involved in recognising and binding of RNA polymerase and other proteins, thereby directing transcription of an operably linked nucleic acid. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences. The term “regulatory element” also encompasses a synthetic fusion molecule or derivative that confers, activates or enhances expression of a nucleic acid molecule in a cell, tissue or organ.

A “plant promoter” comprises regulatory elements, which mediate the expression of a coding sequence segment in plant cells. Accordingly, a plant promoter need not be of plant origin, but may originate from viruses or micro-organisms, for example from viruses which attack plant cells. The “plant promoter” can also originate from a plant cell, e.g. from the plant which is transformed with the nucleic acid sequence to be expressed in the inventive process and described herein. This also applies to other “plant” regulatory signals, such as “plant” terminators. The promoters upstream of the nucleotide sequences useful in the methods of the present invention can be modified by one or more nucleotide substitution(s), insertion(s) and/or deletion(s) without interfering with the functionality or activity of either the promoters, the open reading frame (ORF) or the 3′-regulatory region such as terminators or other 3′ regulatory regions which are located away from the ORF. It is furthermore possible that the activity of the promoters is increased by modification of their sequence, or that they are replaced completely by more active promoters, even promoters from heterologous organisms. For expression in plants, the nucleic acid molecule must, as described above, be linked operably to or comprise a suitable promoter which expresses the gene at the right point in time and with the required spatial expression pattern. The term “operably linked” as used herein refers to a functional linkage between the promoter sequence and the gene of interest, such that the promoter sequence is able to initiate transcription of the gene of interest.

A “constitutive promoter” refers to a promoter that is transcriptionally active during most, but not necessarily all, phases of growth and development and under most environmental conditions, in at least one cell, tissue or organ. Examples of constitutive promoters include but are not limited to actin, HMGP, CaMV19S, GOS2, rice cyclophilin, maize H3 histone, alfalfa H3 histone, 34S FMV, rubisco small subunit, OCS, SAD1, SAD2, nos, V-ATPase, super promoter, G-box proteins and synthetic promoters.

A “strong promoter” refers to a promoter that leads to increased or overexpression of the gene. Examples of strong promoters include, but are not limited to, CaMV-35S, CaMV-35Somega, Arabidopsis ubiquitin UBQ1, rice ubiquitin, actin, or Maize alcohol dehydrogenase 1 promoter (Adh-1). The term “increased expression” or “overexpression” as used herein means any form of expression that is additional to the control, for example wild-type, expression level. In one embodiment, the promoter is CaMV-35S.

In a one embodiment, the promoter is a constitutive or strong promoter. In a preferred embodiment, the regulatory sequence is an inducible promoter, a stress inducible promoter or a tissue specific promoter. The stress inducible promoter is selected from the following non limiting list: the HaHB1 promoter, RD29A (which drives drought inducible expression of DREB1A), the maize rabl7 drought-inducible promoter, P5CS1 (which drives drought inducible expression of the proline biosynthetic enzyme P5CS1), ABA- and drought-inducible promoters of Arabidopsis clade A PP2Cs (ABI1, ABI2, HAB1, PP2CA, HAI1, HAI2 and HAI3) or their corresponding crop orthologs.

In one embodiment, the promoter is not a stress inducible promoter. The promoter may also be tissue-specific.

Other regulatory sequences, such as terminator sequences may also be included.

The invention also relates to an isolated host cell transformed with a nucleic acid or vector as described above. The host cell may be a bacterial cell, such as Agrobacterium tumefaciens, or an isolated plant cell. The invention also relates to a culture medium or kit comprising a culture medium and an isolated host cell as described above.

The mutant nucleic acid or vector described above is used to generate transgenic plants using transformation methods known in the art. Thus, according to the various aspects of the invention, a nucleic acid comprising a sequence encoding for a mutant PYL/PYR polypeptide as described herein, for example a mutant PYL4 with reference to the wild type nucleic acid sequence as shown in SEQ ID No. 1, 2 or 4 is introduced into a plant and expressed as a transgene. The nucleic acid sequence is introduced into said plant through a process called transformation. The terms “introduction” or “transformation” as referred to herein encompass the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

The transfer of foreign genes into the genome of a plant is called transformation. Transformation of plants is now a routine technique in many species. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts, electroporation of protoplasts, microinjection into plant material, DNA or RNA-coated particle bombardment, infection with (non-integrative) viruses and the like. Transgenic plants, including transgenic crop plants, are preferably produced via Agrobacterium tumefaciens mediated transformation.

To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility is growing the seeds, if appropriate after sterilization, on agar plates using a suitable selection agent so that only the transformed seeds can grow into plants. Alternatively, the transformed plants are screened for the presence of a selectable marker such as the ones described above. Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

Thus, the invention relates to a transgenic plant comprising and expressing a mutant nucleic acid, nucleic acid construct comprising a mutant nucleic acid or a vector comprising a mutant nucleic acid wherein said mutant nucleic acid is a nucleic acid of the invention encoding a polypeptide of the invention as described herein. In one embodiment, the mutant nucleic acid encodes a mutant PYL/PYR, for example PYL4, polypeptide comprising a sequence as shown in SEQ ID NO:3, a functional variant or homolog thereof but which comprises an amino acid substitution at a position

a) corresponding to one or more of position A194, V97, C176 and/or F130 in PYL4 as set forth in SEQ ID NO:3 or
b) corresponding to positions H82 and V97 in PYL4 as set forth in SEQ ID NO:3.

Thus, the transgenic plant expresses a mutant version of SEQ ID NO: 3 with one or more mutation as described above (for example any of SEQ ID NOs:60-65) or expresses a mutant which is a homolog/ortholog of AtPYL4.

In one embodiment, the transgenic plant comprises and expresses a mutant nucleic acid encoding a mutant PYL/PYR, for example PYL4, polypeptide comprising a sequence as shown in SEQ ID. NO:3, a functional variant or homolog thereof but which comprises an amino acid substitution at a position A194. In one embodiment, the transgenic plant comprises and expresses a mutant nucleic acid encoding a mutant PYL4 polypeptide comprising a sequence as shown in SEQ ID NO:3, a functional variant or homolog thereof but which comprises an amino acid substitution at a position V97 or a position corresponding thereto. In one embodiment, the transgenic plant comprises and expresses a mutant nucleic acid encoding a mutant PYL4 polypeptide comprising a sequence as shown in SEQ ID NO:3, a functional variant or homolog thereof but which comprises an amino acid substitution at position C176 or a position corresponding thereto. In one embodiment, the transgenic plant comprises and expresses a mutant nucleic acid encoding a mutant PYL4 polypeptide comprising a sequence as shown in SEQ ID. NO:3, a functional variant or homolog thereof but which comprises an amino acid substitution at position F130 or a position corresponding thereto. In one embodiment, the transgenic plant comprises and expresses a mutant nucleic acid encoding a mutant PYL4 polypeptide comprising a sequence as shown in SEQ ID NO:3, a functional variant or homolog thereof but which comprises an amino acid substitution at positions H82 and V97 or at positions corresponding thereto. As explained above with reference to nucleic acids of the invention, any combinations of mutations at positions A194, V97, C176 and/or F130 in PYL4 as set forth in SEQ ID NO:3 are within the scope of the invention.

Thus, the sequence of the AtPYL4 mutant polypeptide is substantially identical to SEQ ID NO:3, but comprises an amino acid substitution at one or more of the positions above. As explained elsewhere, transgenic plants comprising a mutant nucleic acid, nucleic acid construct comprising a mutant nucleic acid or vector comprising a mutant nucleic acid wherein said nucleic acid encodes an ortholog of AtPYL4 with one or more mutation at one or more or corresponding position with reference to SEQ ID NO:3, are also within the scope of the invention. In one embodiment, the transgenic plant comprises and expresses a mutant nucleic acid which encodes a polypeptide that comprises an amino acid substitution corresponding to position A194 with reference to SEQ ID NO:3. In one embodiment, the polypeptide does therefore not comprise any additional activating mutations. In one embodiment, the polypeptide does not have mutations at the following positions with reference to SEQ ID NO:3: H82, V105, V106, L109, A111, D177, F178, V181, C185 and/or S189. Also excluded are specific activating mutations as disclosed in WO 2013/006263. In another embodiment, the polypeptide does not comprise any additional mutations. The A residue at position 194 may be substituted with V, L, M, I or S. In a preferred embodiment, the substitution is A194T. In one embodiment, the mutant polypeptide is PYL4, a functional variant, homolog or ortholog thereof, for example as shown in FIG. 8 or as shown in SEQ ID NOs: 7, 9, 12, 14, 16, 19, 21, 23, 26, 28, 31, 34, 37, 39 or 42 with mutations at corresponding positions.

In another embodiment, the transgenic plant comprises and expresses a mutant nucleic acid which comprises an amino acid substitution corresponding to position H82 and an amino acid substitution corresponding to position V97 with reference to SEQ ID NO:3. Preferably, the polypeptide does not comprise any additional activating mutations. In one embodiment, the polypeptide does not have mutations at the following positions with reference to SEQ ID NO:3: V105, V106, L109, A111, D177, F178, V181, C185 and/or S189. Also excluded are specific activating mutations as disclosed in WO 2013/006263. In another embodiment, the polypeptide does not comprise any additional mutations. The H residue at position 82 may be substituted with K, N, Q, F, Y, W or P. In a preferred embodiment, the substitution is H82R. The V residue at position 97 may be substituted with L, M, I, S, T or A. In one embodiment, the mutant polypeptide is PYL4, a functional variant, homolog or ortholog thereof, for example as shown in FIG. 8 or as shown in SEQ ID NOs: 7, 9, 12, 14, 16, 19, 21, 23, 26, 28, 31, 34, 37, 39 or 42 with mutations at corresponding positions.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

Thus, the invention relates to a method for producing a transgenic plant as described above with improved stress resistance comprising incorporating a nucleic acid encoding for a mutant PYL/PYR polypeptide as defined in SEQ ID NO:3, a functional variant, homolog or ortholog thereof but which comprises a substitution of one or more amino acid corresponding to A194, V97, F130 and/or 0176 in PLY4 as set forth in SEQ ID NO:3 or a substitution of an amino acid corresponding to H82 and of an amino acid corresponding to V97 in PLY4 as set forth in SEQ ID NO:3 into a plant cell by means of transformation, and regenerating the plant from one or more transformed cells. Mutant polypeptides for use in this method are described elsewhere herein. In one embodiment, the PYL/PYR polypeptide is a PLY4 polypeptide as shown in FIG. 8. In another embodiment, the modification is at a position corresponding to A194 in AtPYL4 and the PYL/PYR polypeptide is a PYL4 polypeptide as shown in FIG. 8 or as shown in SEQ ID NOs: 7, 9, 12, 14, 16, 19, 21, 23, 26, 28, 31, 34, 37, 39 or 42.

Another aspect of the invention provides a plant produced by a method described herein which displays improved stress resistance compared to control plant.

Control plants as defined herein are plants that do not express the nucleic acid or construct described above, for example wild type plants or 35S::PYL4 plants.

The plant of the various aspects of the invention is characterised in that it shows increased stress resistance, in particular to drought.

The invention also relates to a method for improving stress resistance or tolerance of a plant, for example drought resistance, comprising incorporating a nucleic acid encoding for a mutant PYL/PYR polypeptide as defined in SEQ ID NO:3, 43-54, a functional variant, homolog or ortholog of any of these sequences but which comprises a substitution of one or more amino acid corresponding to A194, V97, F130 and/or C176 in PLY4 as set forth in SEQ ID NO:3 or a substitution of an amino acid corresponding to H82 and a substitution of an amino acid corresponding to V97 in PLY4 as set forth in SEQ ID NO:3 into a plant cell by means of transformation, and; regenerating the plant from one or more transformed cells. Mutant polypeptides for use in this method are described elsewhere herein. In one embodiment, the PYL/PYR polypeptide is a PLY4 polypeptide, for example as shown in FIG. 8. In another embodiment, the modification is at position A194 and the PYL/PYR polypeptide is a PYL4 polypeptide for example as shown in FIG. 8.

The stress is preferably abiotic stress and may be selected from drought, salinity, freezing (caused by temperatures below 0° C.), chilling (caused by low temperatures over 0° C.) and heat stress (caused by high temperatures). Preferably, the stress is drought.

The stress may be severe or preferably moderate stress. In Arabidopsis research, stress is often assessed under severe conditions that are lethal to wild type plants. For example, drought tolerance is assessed predominantly under quite severe conditions in which plant survival is scored after a prolonged period of soil drying. However, in temperate climates, limited water availability rarely causes plant death, but restricts biomass and seed yield. Moderate water stress, that is suboptimal availability of water for growth can occur during intermittent intervals of days or weeks between irrigation events and may limit leaf growth, light interception, photosynthesis and hence yield potential. Leaf growth inhibition by water stress is particularly undesirable during early establishment. There is a need for methods for making plants with increased yield under moderate stress conditions. In other words, whilst plant research in making stress tolerant plants is often directed at identifying plants that show increased stress tolerance under severe conditions that will lead to death of a wild type plant, these plants do not perform well under moderate stress conditions and often show growth reduction which leads to unnecessary yield loss. Thus, in one embodiment of the methods of the invention, yield is improved under moderate stress conditions. The transgenic plants according to the various aspects of the invention show enhanced tolerance to these types of stresses compared to a control plant and are able to mitigate any loss in yield/growth. The tolerance can therefore be measured as an increase in yield as shown in the examples. The terms moderate or mild stress/stress conditions are used interchangeably and refer to non-severe stress. In other words, moderate stress, unlike severe stress, does not lead to plant death. Under moderate, that is non-lethal, stress conditions, wild type plants are able to survive, but show a decrease in growth and seed production and prolonged moderate stress can also result in developmental arrest. The decrease can be at least 5%-50% or more. Tolerance to severe stress is measured as a percentage of survival, whereas moderate stress does not affect survival, but growth rates. The precise conditions that define moderate stress vary from plant to plant and also between climate zones, but ultimately, these moderate conditions do not cause the plant to die. With regard to high salinity for example, most plants can tolerate and survive about 4 to 8 dS/m. Specifically, in rice, soil salinity beyond ECe˜4 dS/m is considered moderate salinity while more than 8 dS/m becomes high. Similarly, pH 8.8-9.2 is considered as non-stress while 9.3-9.7 as moderate stress and equal or greater than 9.8 as higher stress.

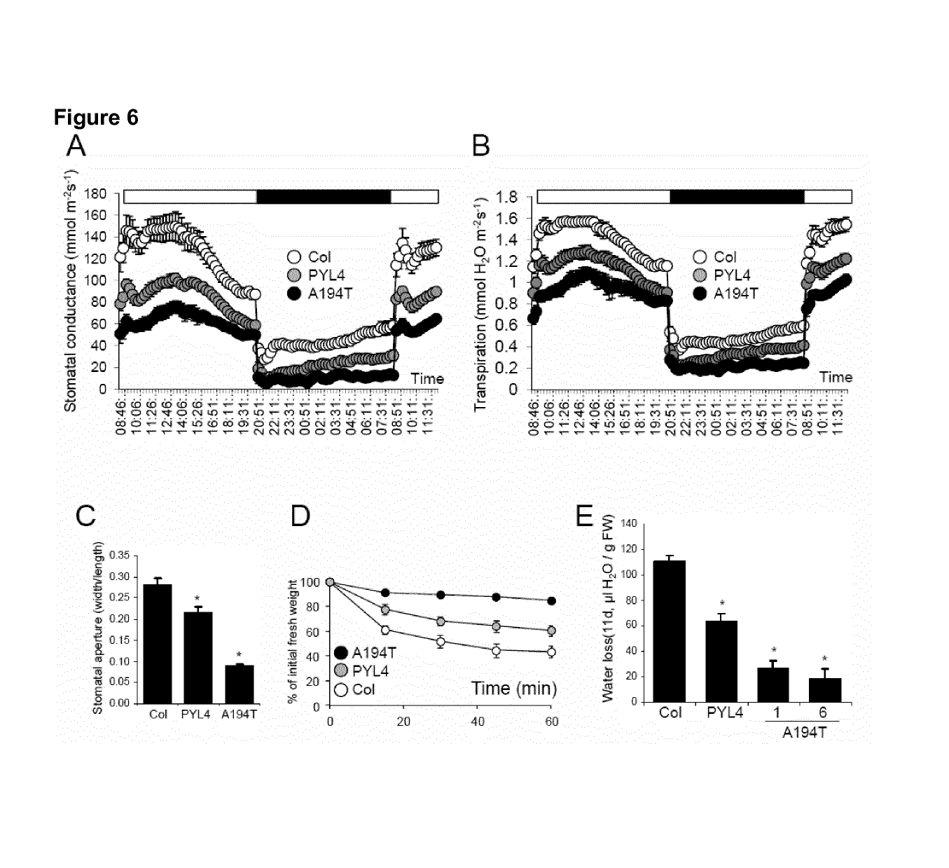

Drought stress can be measured through leaf water potentials. Generally speaking, moderate drought stress is defined by a water potential of between −1 and −2 Mpa. Moderate temperatures vary from plant to plant and specially between species. Normal temperature growth conditions for Arabidopsis are defined at 22-24° C. For example, at 28° C., Arabidopsis plants grow and survive, but show severe penalties because of “high” temperature stress associated with prolonged exposure to this temperature. However, the same temperature of 28° C. is optimal for sunflower, a species for which 22° C. or 38° C. causes mild, but not lethal stress. In other words, for each species and genotype, an optimal temperature range can be defined as well as a temperature range that induces mild stress or severe stress which leads to lethality. Drought tolerance can be measured using methods known in the art, for example assessing survival of the transgenic plant compared to a control plant, or by determining turgor pressure, rosette radius, water loss in leaves, growth or yield. Regulation of stomatal aperture by ABA is a key adaptive response to cope with drought stress. Thus, drought resistance can also be measured by assessing stomatal conductance (Gst) and transpiration in whole plants under basal conditions (see FIGS. 6A and B).

According to the invention, a transgenic plant has enhanced drought tolerance if the survival rates are at least 2, 3, 4, 5, 6, 7, 8, 9 or 10-fold higher than those of the control plant after exposure to drought and/or after exposure to drought and re-watering. Also according to the invention, a transgenic plant has enhanced drought tolerance if the rosette radius is at least 10, 20, 30, 40, 50% larger than that of the control plant after exposure to drought and/or after exposure to drought and re-watering. The plant may be deprived of water for 10-30, for example 20 days and the re-watered. Also according to the invention, a transgenic plant has enhanced drought tolerance if stomatal conductance (Gst) and transpiration are lower than in the control plant, for example at least 10, 20, 30, 40, 50% lower.

Thus in one embodiment, the methods of the invention relate to increasing resistance to moderate (non-lethal) stress or severe stress. In the former embodiment, transgenic plants according to the invention show increased resistance to stress and therefore, the plant yield is not or less affected by the stress compared to wild type yields which are reduced upon exposure to stress. In other words, an improve in yield under moderate stress conditions can be observed.

In one embodiment, the method relates to improving drought tolerance of plant vegetative tissue.

The terms “increase”, “improve” or “enhance” are interchangeable. Yield for example is increased by at least a 3%, 4%, 5%, 6%, 7%, 8%, 9% or 10%, preferably at least 15% or 20%, more preferably 25%, 30%, 35%, 40% or 50% or more in comparison to a control plant. The term “yield” in general means a measurable produce of economic value, typically related to a specified crop, to an area, and to a period of time. Individual plant parts directly contribute to yield based on their number, size and/or weight, or the actual yield is the yield per square meter for a crop and year, which is determined by dividing total production (includes both harvested and appraised production) by planted square meters. The term “yield” of a plant may relate to vegetative biomass (root and/or shoot biomass), to reproductive organs, and/or to propagules (such as seeds) of that plant. Thus, according to the invention, yield comprises one or more of and can be measured by assessing: increased seed yield per plant, increased seed filling rate, increased number of filled seeds, increased harvest index, increased number of seed capsules/pods, increased seed size, increased growth or increased branching, for example inflorescences with more branches. Preferably, increased yield comprises an increased number of seed capsules/pods and/or increased branching. Yield is increased relative to control plants.

Thus, the invention also relates to improving yield under stress conditions, preferably moderate stress conditions, comprising incorporating a nucleic acid encoding for a mutant PYL/PYR polypeptide as defined in SEQ ID NO:3, a functional variant, homolog or ortholog thereof but which comprises a substitution of one or more amino acid corresponding to A194, V97, F130 and/or C176 in PLY4 as set forth in SEQ ID NO:3 or a substitution of an amino acid corresponding to H82 and a substitution of an amino acid corresponding to V97 in PLY4 as set forth in SEQ ID NO:3 into a plant cell by means of transformation, and; regenerating the plant from one or more transformed cells. Mutant polypeptides for use in this method are described elsewhere herein. In one embodiment, the PYL/PYR polypeptide is a PLY4 polypeptide as shown in FIG. 8 or as shown in SEQ ID NOs: 7, 9, 12, 14, 16, 19, 21, 23, 26, 28, 31, 34, 37, 39 or 42 with one or more corresponding mutation as shown for AtPYL4. In another embodiment, the modification is at a position corresponding to A194 in AtPYL4 and the PYL/PYR polypeptide is a PYL4 polypeptide as shown in FIG. 8 or as shown in SEQ ID NOs: 7, 9, 12, 14, 16, 19, 21, 23, 26, 28, 31, 34, 37, 39 or 42 with a corresponding mutation.

The various aspects of the invention described herein clearly extend to any plant cell or any plant produced, obtained or obtainable by any of the methods described herein, and to all plant parts and propagules thereof unless otherwise specified. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

The invention also extends to harvestable parts of a plant of the invention as described above such as, but not limited to seeds, leaves, fruits, flowers, stems, roots, rhizomes, tubers and bulbs. The invention furthermore relates to products derived, preferably directly derived, from a harvestable part of such a plant, such as dry pellets or powders, oil, fat and fatty acids, starch or proteins. The invention also relates to food products and food supplements comprising the plant of the invention or parts thereof.

The transgenic plant according to the various aspects of the invention described herein may be a moncot or a dicot plant. The plant PYL/PYR nucleic acid/polypeptide is a monocot or dicot PYL/PYR nucleic acid/polypeptide. Non-limiting examples of moncot or a dicot plants are given below.

A dicot plant may be selected from the families including, but not limited to Asteraceae, Brassicaceae (eg Brassica napus), Chenopodiaceae, Cucurbitaceae, Leguminosae (Caesalpiniaceae, Aesalpiniaceae Mimosaceae, Papilionaceae or Fabaceae), Malvaceae, Rosaceae or Solanaceae. For example, the plant may be selected from lettuce, sunflower, Arabidopsis, broccoli, spinach, water melon, squash, cabbage, tomato, potato, yam, capsicum, tobacco, cotton, okra, apple, rose, strawberry, alfalfa, bean, soybean, field (fava) bean, pea, lentil, peanut, chickpea, apricots, pears, peach, grape vine or citrus species. In one embodiment, the plant is oilseed rape.

Also included are biofuel and bioenergy crops such as rape/canola, sugar cane, sweet sorghum, Panicum virgatum (switchgrass), linseed, lupin and willow, poplar, poplar hybrids, Miscanthus or gymnosperms, such as loblolly pine. Also included are crops for silage (maize), grazing or fodder (grasses, clover, sanfoin, alfalfa), fibres (e.g. cotton, flax), building materials (e.g. pine, oak), pulping (e.g. poplar), feeder stocks for the chemical industry (e.g. high erucic acid oil seed rape, linseed) and for amenity purposes (e.g. turf grasses for golf courses), ornamentals for public and private gardens (e.g. snapdragon, petunia, roses, geranium, Nicotiana sp.) and plants and cut flowers for the home (African violets, Begonias, chrysanthemums, geraniums, Coleus spider plants, Dracaena, rubber plant).

A monocot plant may, for example, be selected from the families Arecaceae, Amaryllidaceae or Poaceae. For example, the plant may be a cereal crop, such as wheat, rice, barley, maize, oat, sorghum, rye, millet, buckwheat, turf grass, Italian rye grass, sugarcane or Festuca species, or a crop such as onion, leek, yam or banana.

Preferably, the plant is a crop plant. By crop plant is meant any plant which is grown on a commercial scale for human or animal consumption or use.

Most preferred plants are maize, rice, wheat, oilseed rape, sorghum, soybean, potato, tomato, tobacco, grape, barley, pea, bean, field bean, lettuce, cotton, sugar cane, sugar beet, broccoli or other vegetable brassicas or poplar.

Sequences for a non-limiting list of preferred PYL4 orthologs are shown as SEQ ID NOs: 7, 9, 12, 14, 16, 19, 21, 23, 26, 28, 31, 34, 37, 39 or 42, but when used according to the invention, these have mutations at positions corresponding to those as set out for SEQ ID NO:3 herein. For example, a nucleic acid encoding SEQ ID No. 28 with one or more corresponding mutation may be introduced and expressed in rice, a nucleic acid encoding SEQ ID NO:19 with one or more corresponding mutation may be introduced and expressed in soybean, a nucleic acid encoding SEQ ID NO:14 with one or more corresponding mutation may be introduced and expressed in tobacco, a nucleic acid encoding SEQ ID NO:34 with one or more corresponding mutation may be introduced and expressed in maize or a nucleic acid encoding SEQ ID NO:31 with one or more corresponding mutation may be introduced and expressed in barley. Alternatively, the plant is any of the plants defined herein, preferably a crop plant such as maize, wheat, oilseed rape, sorghum, soybean, potato, tomato, tobacco, grape, barley, pea, bean, field bean, lettuce, cotton, sugar cane, sugar beet, broccoli or other vegetable brassicas or poplar and the sequence expressed is a nucleic acid sequence encoding a mutant of SEQ ID NO:3 as defined herein.

The term “plant” as used herein encompasses whole plants, ancestors and progeny of the plants and plant parts, including seeds, fruit, shoots, stems, leaves, roots (including tubers), flowers, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprises the gene/nucleic acid of interest.

The invention also relates to the use of an isolated nucleic acid, nucleic acid construct or vector as described herein in increasing stress resistance, for example to drought, and/or yield of a plant. The invention also relates to the use of an isolated mutant nucleic acid, nucleic acid construct or vector as described herein in reducing stomatal conductance. The invention also relates to the use of an isolated nucleic acid, nucleic acid construct or vector as described herein in increasing water use efficiency. The term water use efficiency as used herein relates to the plants ability of using a water supply efficiently under normal or water deficit conditions. The invention also relates to corresponding methods, that is methods for increasing stress resistance, reducing stomatal conductance, increasing water use efficiency in a transgenic plant comprising introducing and expressing a nucleic acid encoding for a mutant PYL/PYR polypeptide as defined in SEQ ID NO:3, a functional variant, homolog or ortholog thereof but which comprises a substitution of one or more of the amino acid corresponding to A194, V97, F130 and/or C176 in PLY4 as set forth in SEQ ID NO:3 or a position corresponding thereto or a substitution of an amino acid corresponding to H82 and a substitution of an amino acid corresponding to V97 in PYL4 as set forth in SEQ ID NO:3 or a position corresponding thereto into a plant. Preferably, the method is carried out at low ABA levels.

The invention also relates to a method for prolonging seed dormancy/preventing early germination/induce hyperdormancy in a transgenic plant comprising introducing and expressing a nucleic acid encoding for a mutant PYL/PYR polypeptide as defined in SEQ ID NO:3, a functional variant, homolog or ortholog thereof but which comprises a substitution of one or more of the amino acid corresponding to A194, V97, F130 and/or C176 in PLY4 as set forth in SEQ ID NO:3 or a substitution of an amino acid corresponding to H82 and a substitution of an amino acid corresponding to V97 in PLY4 as set forth in SEQ ID NO:3 into a plant. Mutant polypeptides for use in this method are described elsewhere herein. In one embodiment, the PYL/PYR polypeptide is a PLY4 polypeptide as shown in FIG. 8 or SEQ ID NOs: 7, 9, 12, 14, 16, 19, 21, 23, 26, 28, 31, 34, 37, 39 or 42 with one or more mutation at corresponding position. In another embodiment, the modification is at positions A194 and the PYL/PYR polypeptide is a PYL4 polypeptide as shown in FIG. 8 or as shown in SEQ ID NOs: 7, 9, 12, 14, 16, 19, 21, 23, 26, 28, 31, 34, 37, 39 or 42 with a mutation at corresponding position.

The invention also relates to a method for constitutive activation of the ABA signalling pathway comprising PLY4 receptor comprising introducing and expressing a nucleic acid encoding for a mutant PYL/PYR polypeptide as defined in SEQ ID NO:3, a functional variant, homolog or ortholog thereof but which comprises a substitution of one or more amino acid corresponding to A194, V97, F130 and/or C176 in PLY4 as set forth in SEQ ID NO:3 or a substitution of an amino acid corresponding to H82 and a substitution of an amino acid corresponding to V97 in PLY4 as set forth in SEQ ID NO:3 into a plant. Mutant polypeptides for use in this method are described elsewhere herein. In one embodiment, the PYL/PYR polypeptide is a PLY4 polypeptide as shown in FIG. 8 or SEQ ID NOs: 7, 9, 12, 14, 16, 19, 21, 23, 26, 28, 31, 34, 37, 39 or 42 with one or more mutation at corresponding position. In another embodiment, the modification is at positions A194 and the PYL/PYR polypeptide is a PYL4 polypeptide as shown in FIG. 8 or as shown in SEQ ID NOs: 7, 9, 12, 14, 16, 19, 21, 23, 26, 28, 31, 34, 37, 39 or 42 with a mutation at a corresponding position.

The invention also relates to a method for inhibiting the activity of PP2C, preferably PP2CA, in a transgenic plant comprising introducing and expressing a nucleic acid encoding for a mutant PYL/PYR polypeptide as defined in SEQ ID NO:3, a functional variant, homolog or ortholog thereof but which comprises a substitution of an amino acid corresponding to one or more of A194, V97, F130 and/or C176 in PLY4 as set forth in SEQ ID NO:3 or a substitution of an amino acid corresponding to H82 and a substitution of an amino acid corresponding to V97 in PLY4 as set forth in SEQ ID NO:3 into a plant. Mutant polypeptides for use in this method are described elsewhere herein. In one embodiment, the PYL/PYR polypeptide is a PLY4 polypeptide as shown in FIG. 8 or SEQ ID NOs: 7, 9, 12, 14, 16, 19, 21, 23, 26, 28, 31, 34, 37, 39 or 42 with one or more mutation at corresponding position. In another embodiment, the modification is at positions A194 and the PYL/PYR polypeptide is a PYL4 polypeptide as shown in FIG. 8 or as shown in SEQ ID NOs: 7, 9, 12, 14, 16, 19, 21, 23, 26, 28, 31, 34, 37, 39 or 42 with a mutation at a corresponding position.

The invention also relates to a method for improving ABA-dependent inhibition of PP2C, preferably PP2CA, in a transgenic plant comprising, introducing and expressing a mutant nucleic acid encoding for a mutant PYL/PYR polypeptide as defined in SEQ ID NO:3, a functional variant, homolog or ortholog thereof but which comprises a substitution of an amino acid corresponding to one or more of A194, V97, F130 and/or C176 in PLY4 as set forth in SEQ ID NO:3 or a substitution of an amino acid corresponding to H82 and a substitution of an amino acid corresponding to V97 in PLY4 as set forth in SEQ ID NO:3 into a plant. Preferably, inhibition is improved at low ABA levels.

The invention also relates to a method for identifying a mutation in a PYL/PYR polypeptide that confers drought resistance to vegetative tissue compressing mutagenising a plant population, regenerating progeny plants, exposing plants to drought conditions and comparing the phenotype to control plants and plants expressing AtPYL4 with a mutation at position A194. Plants with a phenotype similar to that of plants expressing AtPYL4 are identified and the sequences of PYL/PYR polypeptides are analysed.

In a further aspect, the invention relates to a method for producing a mutant plant expressing a PYR/PYL variant and which is characterised by one of the phenotypes described herein wherein said method uses mutagenesis and Targeting Induced Local Lesions in Genomes (TILLING) to target the gene expressing a PYR/PYL polypeptide. According to this method, lines that carry a specific mutation are produced that has a known phenotypic effect. For example, mutagenesis is carried out using TILLING where traditional chemical mutagenesis is flowed by high-throughput screening for point mutations. This approach does thus not involve creating transgenic plants. The plants are screened for one of the phenotypes described herein, for example a plant that shows increased stress resistance. A PYR/PYL locus is then analysed to identify a specific a PYR/PYL mutation responsible for the phenotype observed. Plants can be bred to obtain stable lines with the desired phenotype and carrying a mutation in a PYR/PYL locus.

Another technique that can be used for targeted DNA editing is Clustered Regularly Interspaced Short Palindromic Repeats (CRISPR) (U.S. Pat. No. 8,697,359, Ran et al incorporated by reference). The CRISPR system can be used to introduce specific nucleotide modifications at the target sequence. Originally discovered in bacteria, where several different CRISPR cascades function as innate immune systems and natural defence mechanisms, the engineered CRISPR-Cas9 system can be programmed to target specific stretches of genetic code and to make cuts at precise locations. Over the past few years, those capabilities have been harnessed and used as genome editing tools, enabling researchers to permanently modify genes in mammalian and plant cells.

Thus, the invention relates to a method for generating a PYL/PYR mutant nucleic acid encoding a mutant PYL or PYR polypeptide comprising an amino acid substitution corresponding to
a) one or more of position A194, V97, C176 and/or F130 in PYL4 as set forth in SEQ ID NO:3 or
b) positions H82 and V97 in PYL4 as set forth in SEQ ID NO:3
   wherein said method comprises modifying a plant endogenous genome using CRISPR.

The method involves targeting of Cas9 to the specific genomic locus, in this case PYL/PYR, via a 20 nt guide sequence of the single-guide RNA. An online CRISPR Design Tool can identify suitable target sites (http://tools.genome-engineering.org, Ren et al).

Plants obtained through such methods are also within the scope of the invention. Thus, the invention relates to a non-transgenic plant obtained by mutagenesis or genome editing comprising and expressing a PYL/PYR nucleic acid which encodes a PYL/PYR mutant polypeptide that has a different sequence compared to the wild type sequence. The mutant polypeptide comprises an amino acid substitutions corresponding to

a) one or more of position A194, V97, C176 and/or F130 in PYL4 as set forth in SEQ ID NO:3 or
b) positions H82 and V97 in PYL4 as set forth in SEQ ID NO:3.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

All documents mentioned in this specification, including all reference to SEQ ID NOs in gene and protein databases are incorporated herein by reference in their entirety. Sequence versions are version 1 unless otherwise specified.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein. Unless context dictates otherwise, the descriptions and definitions of the features set out above are not limited to any particular aspect or embodiment of the invention and apply equally to all aspects and embodiments which are described.

The invention is further described in the following non-limiting examples.

### Examples

### Identification of PYL4 Mutations that Promote ABA-Independent Interaction with PP2CA in Yeast

PYL4 interacts in an ABA-dependent manner with PP2CA in Y2H assays (Lackman et al., 2011; FIG. 1A). We conducted error-prone PCR mutagenesis of the PYL4 receptor and generated an allele library of approximately 10,000 clones in the pGBKT7 vector. The library was shuttled to yeast AH109 by co-transformation with pGAD7-PP2CA. Yeast transformants were pooled and clones able to grow in the absence of exogenous ABA in medium lacking histidine and adenine were selected. Yeast plasmids were extracted, sequenced and retransformed in yeast cells to recapitulate the phenotype. Thus, different mutations in the encoded PYL4 protein were identified that enabled constitutive interaction with PP2CA (FIGS. 1, A and B). Through site-saturation mutagenesis of PYR1, Mosquna et al., (2011) identified mutations located in 10 different residues of the receptor that promoted PYR1-HAB1 interactions in the absence of ABA. These activating mutations were clustered in the receptor-phosphatase interaction surface, specifically in the gating loop of PYR1, its C-terminal α-helix and H60. The H60 of PYR1 is a hotspot for activating mutations, and for instance, the H60P substitution destabilizes the PYR1 dimer and increases its apparent ABA affinity, and both PYR1H60P and PYR1H60R bound HAB1 in the absence of ABA (Dupeux et al., 2011a; Mosquna et al., 2011). The H60 equivalent residue in PYL4 is H82, and interestingly we found in our screening a PYL4H82R mutation that resulted in ABA-independent interaction with PP2CA (FIG. 1A). The H82R mutation was found combined with V97A but the individual V97A mutation did not affect the interaction in the absence of ABA, although it increased yeast growth in the presence of ABA (FIG. 1A). Other mutations that enhanced the interaction of PYL4 and PP2CA in the absence of ABA were A194T and the double mutation F130Y C176R (FIGS. 1, A and B). Both A194T and C176R mutations are located in the C-terminal helix of PYL4, which represented another hotspot for activating mutations in PYR1 since this α-helix forms part of the receptor-phosphatase binding interface (Mosquna et al., 2011). The interaction of the PP2C HAB1 with PYL4 was also found to be ABA-dependent in yeast (Lackman et al., 2011), so we decided to test whether the PYL4 described mutations affected the interaction with HAB1 in the absence of ABA. However, in contrast to their effect on the interaction with PP2CA, these mutant versions of PYL4 behaved mostly as PYL4 when assayed with HAB1 (FIG. 1A).

### Effect of PYL4 Mutations on PP2CA Activity In Vitro

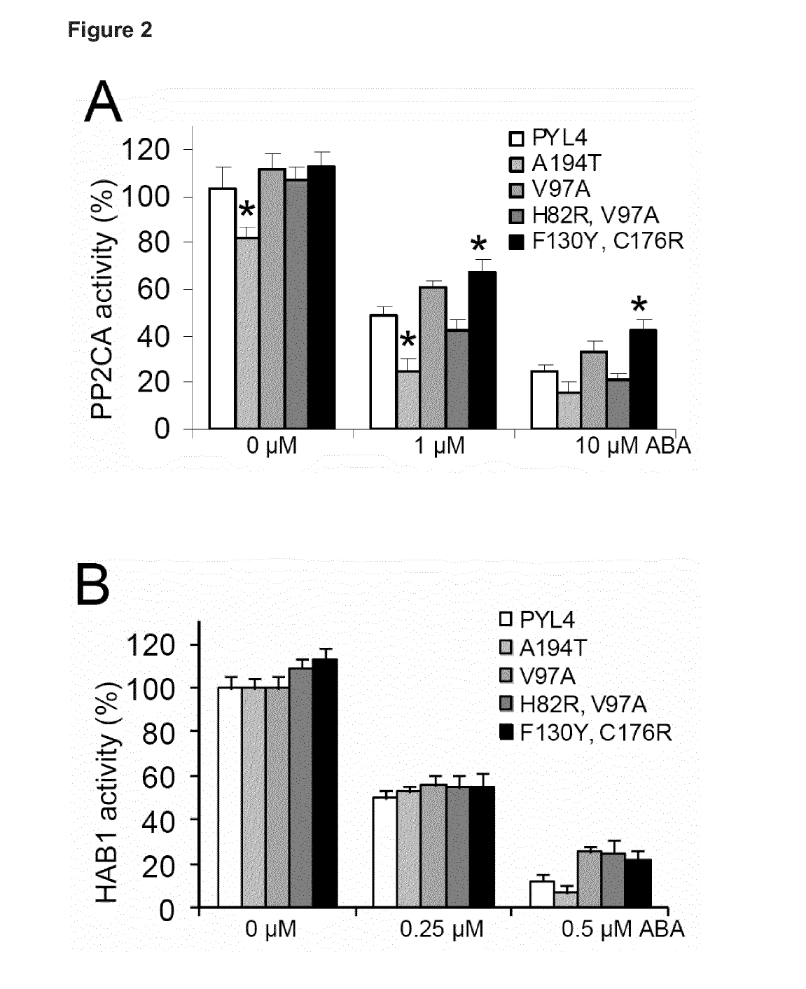

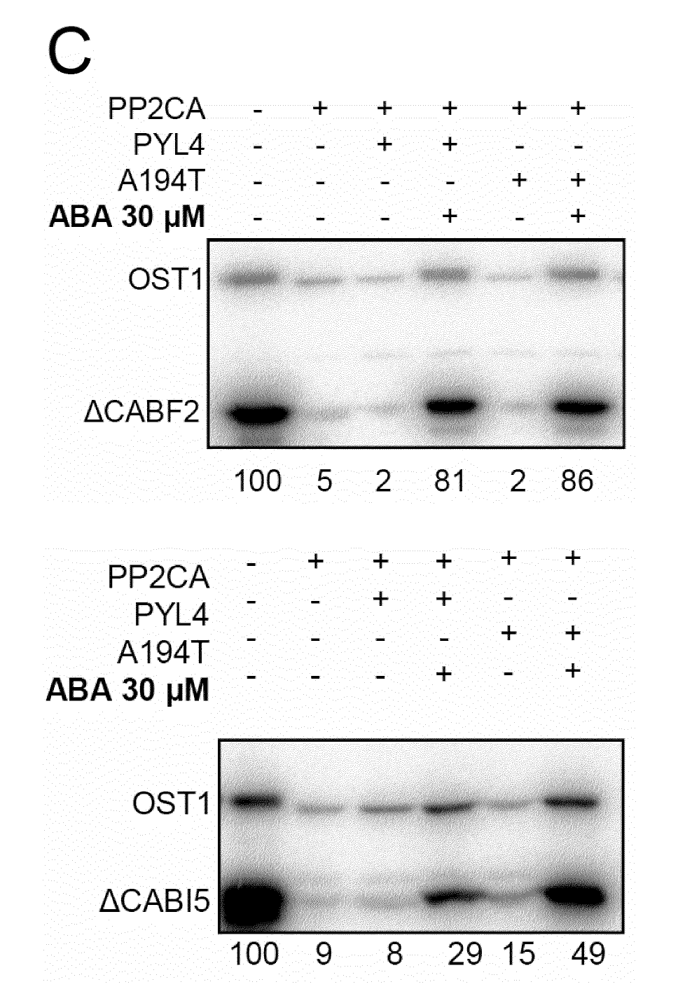

Y2H assays reveal both ABA-independent and -dependent interactions among PYR/PYLs and PP2Cs; however, PYR/PYL receptors inhibit the activity of clade A PP2Cs mostly in an ABA-dependent manner (Park et al., 2009; Ma et al., 2009; Santiago et al., 2009; Fujii et al., 2009). Thus, an ABA-independent interaction in Y2H assay does not necessarily imply capacity to inhibit phosphatase activity in the absence of ABA. Indeed, although most of the monomeric PYR/PYL receptors show ABA-independent interaction with different PP2Cs in Y2H assay, effective phosphatase inhibition requires ABA, and for instance, the in vivo binding of PYL8 to five clade A PP2Cs was largely dependent on ABA (Park et al., 2009; Ma et al., 2009; Santiago et al., 2009; Antoni et al., 2013). Therefore, we tested whether these mutations affected actually the activity of two clade A PP2Cs, i.e. PP2CA and HAB1. Using p-nitrophenyl phosphate (pNPP) as a substrate, we could detect a small inhibitory effect (20%) of PYL4^{A194T} on the activity of PP2CA in the absence of ABA with respect to PYL4 (FIG. 2A). However, although the H82RV97A and F130Y C176R mutations promoted ABA-independent interactions in Y2H assay, they did not affect PP2CA activity in the absence of ABA. In the presence of 1 μM ABA, PYL4^{A194T} also showed a higher inhibition of PP2CA than PYL4 (FIG. 2A). The other mutations behaved similarly to PYL4 except F130Y C176R, which showed lower capacity to inhibit PP2CA in the presence of ABA. PYL4 inhibited more efficiently HAB1 than PP2CA (1050 of 0.25 and 1 μM, respectively), and all PYL4 mutants inhibited HAB1 similarly to PYL4 (FIG. 2B).

Although phosphatase activity is usually measured using small phosphorylated molecules such as pNPP or phosphopeptides, in vivo phosphatase activity is addressed against phosphorylated proteins and therefore could involve substrate-dependent effects. Therefore, we also performed in vitro reconstitution of the ABA signaling cascade and measured the capacity of PYL4^{A194T} or PYL4 to inhibit the dephosphorylation of several PP2CA targets, i.e. OST1/SnRK2.6, ΔC-ABF2 (residues 1-173) and ΔC-ABI5 (residues 1-200) transcription factors or N-terminal fragment (residues 1-186) of the anion channel SLAC1 (FIG. 2C). First a phosphorylation reaction was performed where OST1 was autophosphorylated in vitro and in turn it phosphorylated ΔC-ABF2, ΔC-ABI5 and SLAC1¹⁻¹⁸⁶ proteins. Next, these proteins were used as substrates of PP2CA that was pre-incubated (or not) for 10 min with PYL4 or PYL4^{A194T} either in the absence or presence of 30 μM ABA. In the absence of ABA, we did not find significant differences among PYL4 and PYL4^{A194T}. In the presence of 30 μM ABA, PYL4^{A194T} inhibited better than PYL4 the dephosphorylation by PP2CA of ΔC-ABI5 and SLAC1¹⁻¹⁸⁶, although it was not more effective than PYL4 to inhibit the dephosphorylation of ΔC-ABF2 (FIG. 2C). PYL4^{A194T} shows enhanced capacity to interact with PP2CA in the absence of ABA, probably because novel contact points are generated by the mutation. Therefore, we reasoned that this mutation might also lead to enhanced association kinetics in the presence of ABA, particularly at low ABA levels or low phosphatase:receptor ratios. We performed dephosphorylation assays at low ABA concentrations (0.1, 0.5 and 1 μM) and 1:1 phosphatase:receptor ratio (FIG. 2D). In the presence of 0.5-1 μM ABA, PYL4^{A194T} inhibited better (2-3 fold) than PYL4 the dephosphorylation of ΔC-ABF2, ΔC-ABI5 and SLAC1¹⁻¹⁸⁶. Protection of OST1 phosphorylation was also improved by PYL4^{A194T} compared to PYL4 (FIG. 2D).

### In Vitro and In Vivo Interaction of PYL4A194T and PP2CA

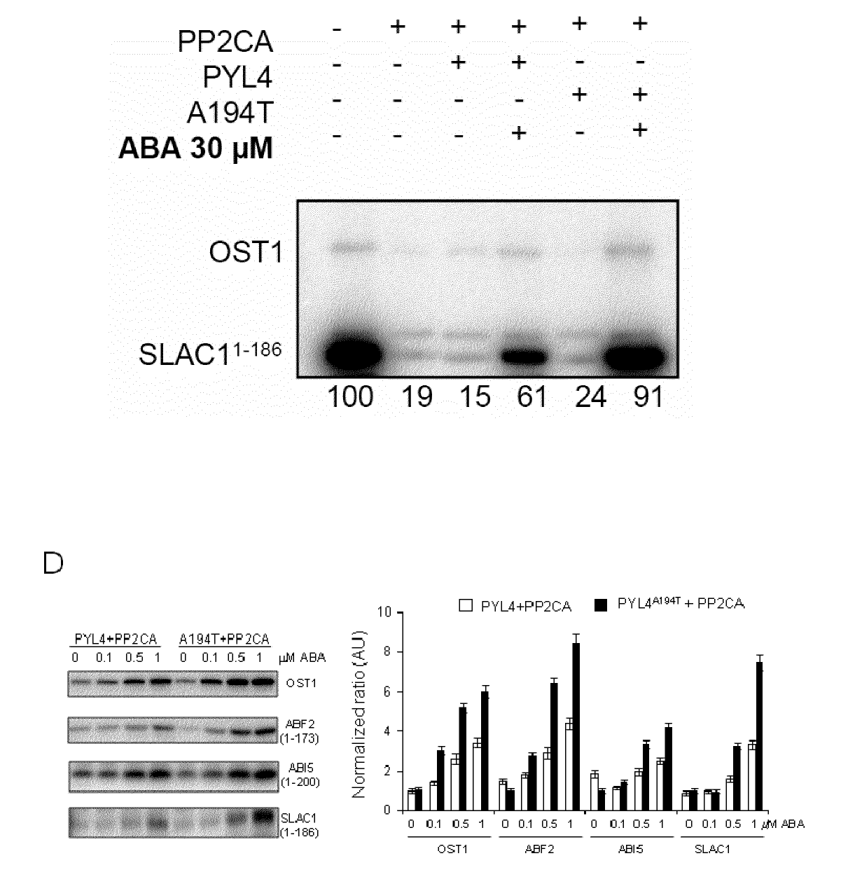

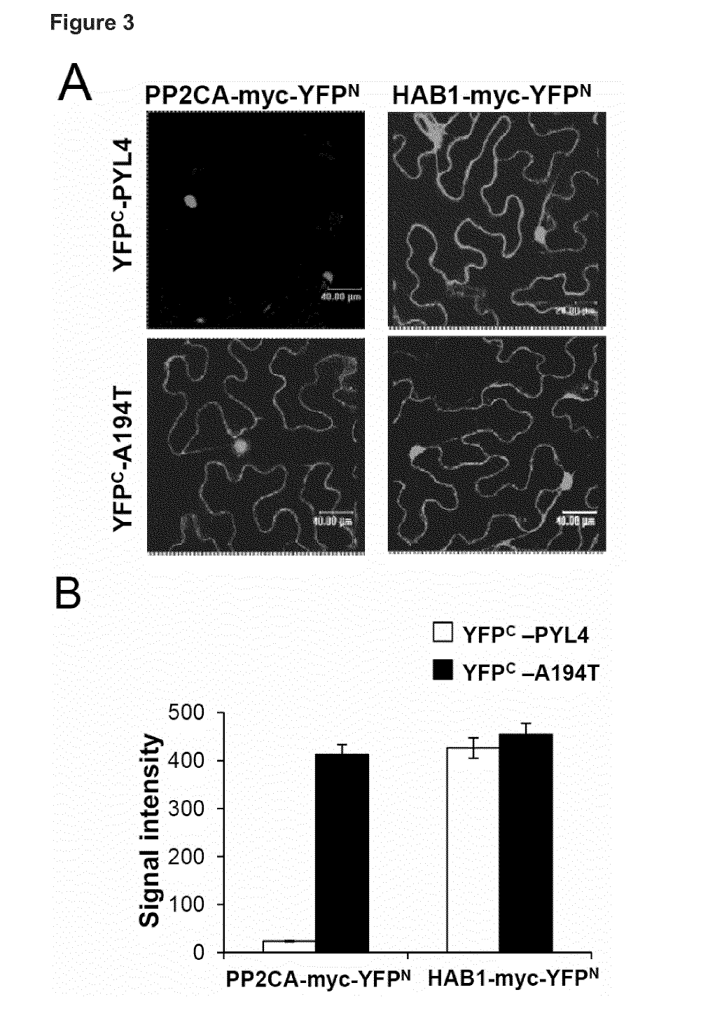

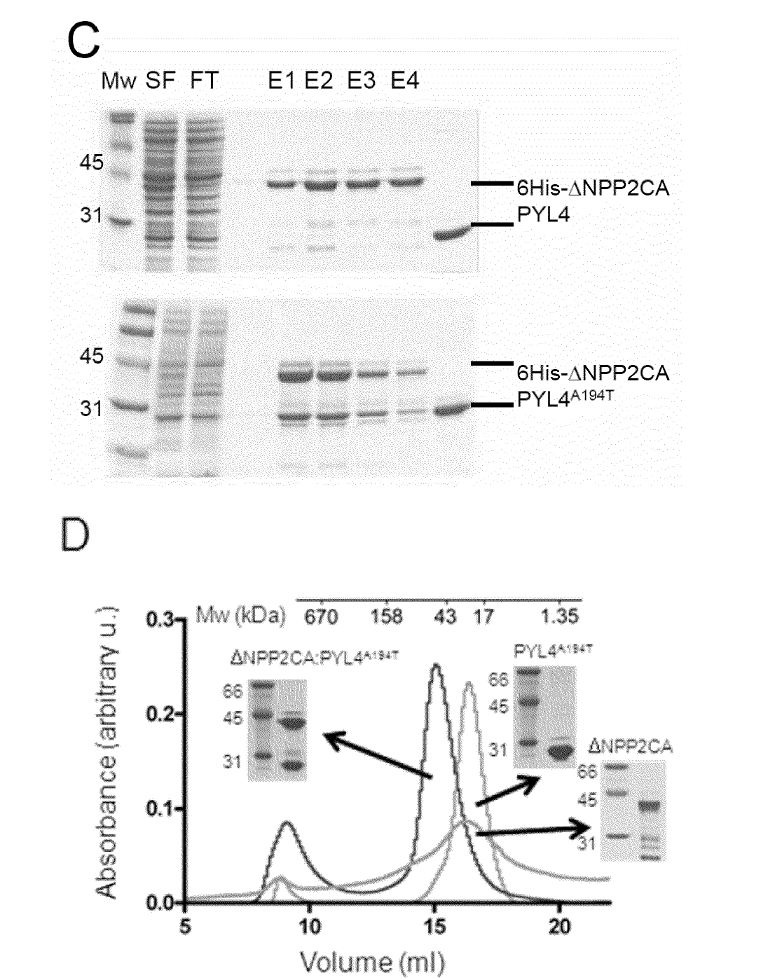

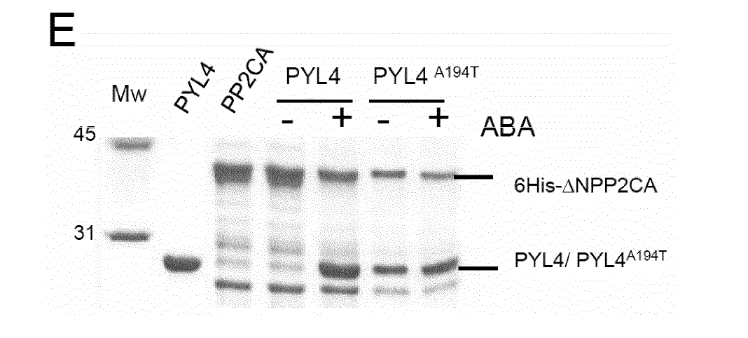

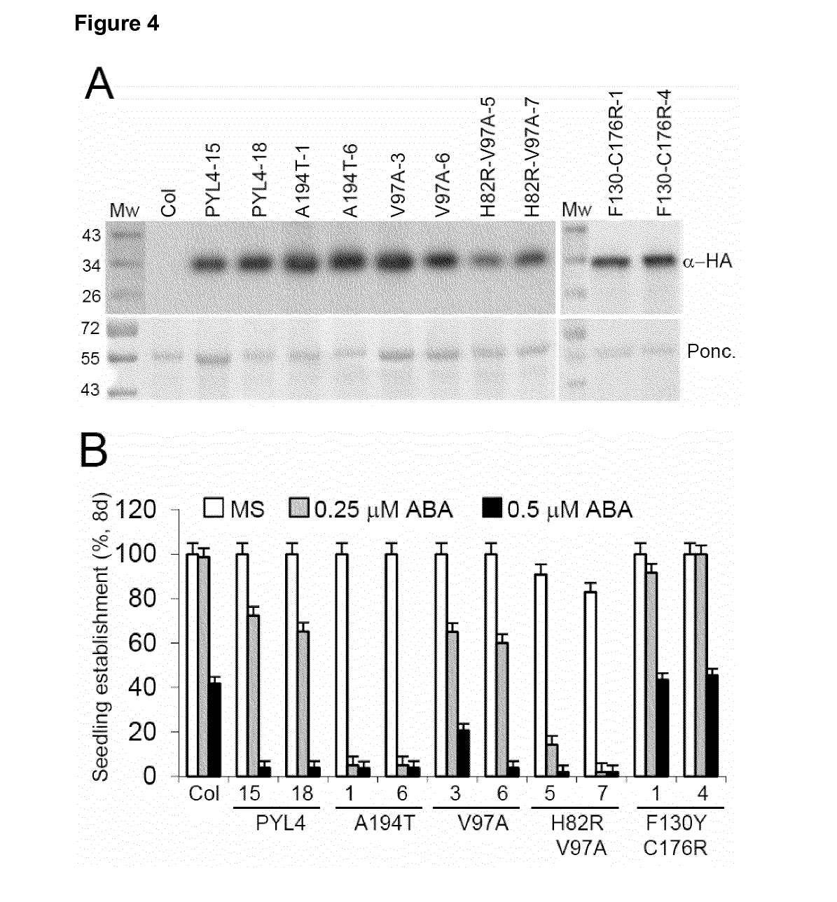

The phenotype described below for 35S: PYL4A194T plants (see FIGS. 4 to 7) prompted us to further analyze the interaction between PYL4A194T and PP2CA using in vivo and in vitro protein-protein interaction tests. First, BiFC assays were used to analyze the interaction of PYL4 or PYL4A194T and either PP2CA or HAB1 in tobacco cells (FIG. 3A). To this end, we performed transient expression of PP2CA-YFPN and YFPC-PYL4 in epidermal cells of N. benthamiana using Agroinfiltration. The interaction between PP2CA and PYL4 did not require the addition of exogenous ABA; endogenous ABA levels in tobacco cells after Agroinfiltration appear to be enough to promote such interaction, which was localized mostly to the nucleus. In contrast, the interaction of PYL4A194T and PP2CA was relatively much more abundant in the cytosol compared to PYL4, and the relative fluorescence emission was higher in the PYL4A194T-PP2CA interaction (FIG. 3B). The interaction of HAB1 and either PYL4 or PYL4A194T did not differ significantly (FIGS. 3A and B).

Next we performed in vitro protein-protein interaction assays. Non-His tagged PYL4^{A194T} could be co-purified with 6His-ΔNPP2CA using Ni-affinity chromatography in the absence of ABA, in contrast to PYL4 (FIG. 3C). Size exclusion chromatography and SDS-PAGE analysis of the eluted fractions confirmed that both proteins formed a 1:1 complex in the absence of ABA (FIG. 3D). Finally, a pull-down assay showed that whereas the interaction of PYL4 and PP2CA was dependent on the addition of ABA, ABA-independent binding could be observed for PYL4A194T and PP2CA (FIG. 3E). Therefore, both in vivo and in vitro assays show a differential interaction of PYL4A194T and PP2CA with respect to PYL4.

### Analysis of Transgenic Lines Over-Expressing PYL4 Mutants

In order to study the putative effect of PYL4 mutations on ABA signaling in vivo, we generated transgenic plants that over-expressed hemmaglutinin (HA)-tagged versions of PYL4 or the mutant versions PYL4V97A, PYL4A194T, PYL4C176R F130Y and PYL4H82R V97A. Expression of the proteins in vegetative tissue was detected by immunoblot analysis and transgenic lines that expressed similar levels of PYL4 and mutant PYL4 proteins were selected for further analysis; however, PYL4H82R V97A lines consistently showed lower expression of the transgene compared to PYL4 or other mutant proteins (FIG. 4A). Over-expression of PYL4 or PYL4V97A enhanced ABA-mediated inhibition of seedling establishment compared to non-transformed plants, whereas ABA sensitivity of PYL4C176R F130Y over-expressing (OE) plants was similar to non-transformed plants. Interestingly, both PYL4A194T and PYL4H82RV97A OE plants showed higher sensitivity to ABA-mediated inhibition of seedling establishment than PYL4 OE plants (FIG. 4B). Low concentrations of ABA (0.25-0.5 μM) delay seedling establishment of non-transformed Col wt and have a limited inhibitory effect on further growth of the seedlings (FIG. 4C). This effect was enhanced in PYL4 or PYL4V97A OE plants, particularly evident at 0.5 μM ABA (FIG. 4C). In the case of PYL4A194T and PYL4H82RV97A OE plants, the effect was even visible at 0.25 μM ABA, indicating that these lines show higher sensitivity to ABA-mediated inhibition of shoot growth than PYL4 OE plants (FIGS. 4C and 4D).

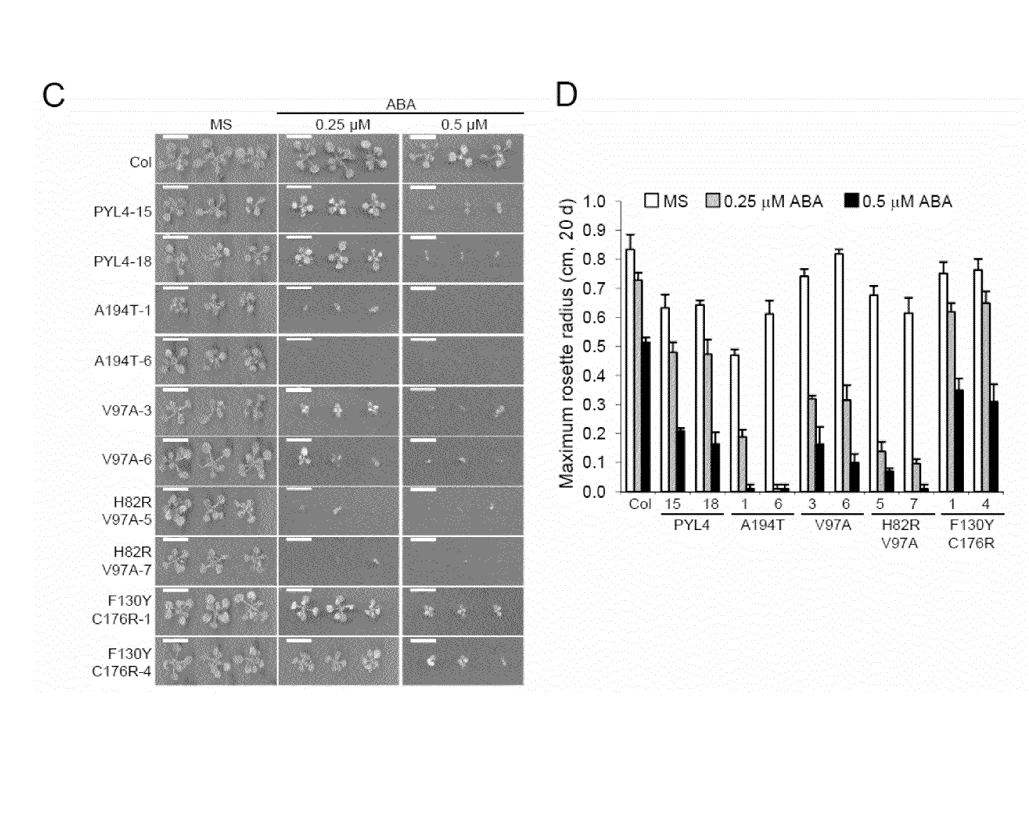

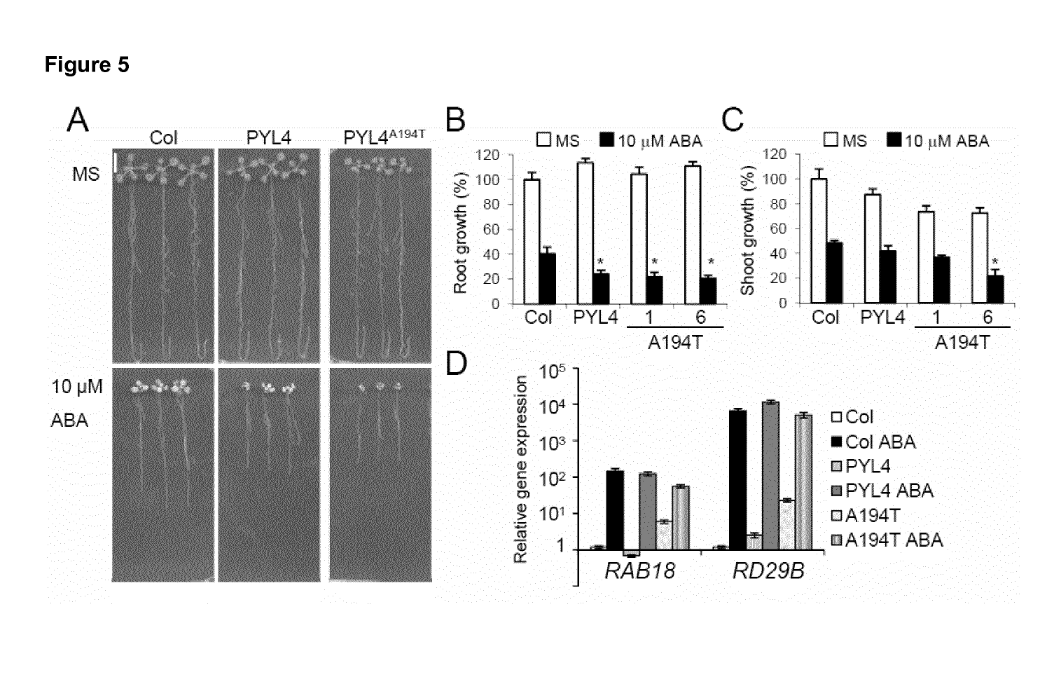

We concentrated further analysis on PYL4A194T transgenic lines, where expression of the transgene remained stable in T4 lines. Seed germination and seedling establishment analyses of PYL4A194T OE lines confirmed the enhanced sensitivity to ABA observed in T3 seeds. Moreover, root and shoot growth analyses also revealed enhanced sensitivity to ABA in vegetative tissues (FIG. 5). We transferred 4-d-old seedlings to MS medium plates lacking or supplemented with 10 μM ABA and root growth was measured 10 d after transfer. Both PYL4 and PYL4A194T OE plants showed enhanced ABA-mediated inhibition of root growth compared to non-transformed plants (FIGS. 5, A and B). Shoot growth was evaluated by measuring the maximum rosette radius of plants grown for 11 d in MS medium lacking or supplemented with 10 μM ABA (FIG. 5C). Finally, we measured expression of two ABA-responsive genes, RAB18 and RD29B, in mock- or 10 μM ABA-treated plants (FIG. 5D). In the absence of exogenous ABA treatment, expression of RAB18 and RD29B was 6- and 23-fold, respectively, up-regulated in PYL4A194T OE plants compared to non-transformed plants. These results indicate a partial de-repression of ABA responsive genes in this line compared to non-transformed plants. However, after ABA-treatment, the induction of these genes was not higher than in non-transformed plants.

### PYL4A194T OE Plants Show Enhanced Drought Resistance

Regulation of stomatal aperture by ABA is a key adaptive response to cope with drought stress. In order to probe stomatal function in non-transformed Col, PYL4 and PYL4A194T OE plants, we performed analysis of stomatal conductance (Gst) and transpiration in whole plants under basal conditions (FIGS. 6A and B). Interestingly, both PYL4 and PYL4A194T OE lines showed lower Gst and transpiration values than non-transformed Col plants. Moreover, PYL4A194T OE plants showed lower Gst values than PYL4 OE plants. Diurnal course of Gst was generally not affected in transformed plants; both OE lines closed their stomata like non-transformed Col wt during the night and showed maximum Gst values around mid-day, followed by pre-dark stomatal closure. Still, pre-dawn stomatal opening was more pronounced in non-transformed Col wt and PYL4 OE compared to PYL4A194T OE plants. The latter result could be directly related to an enhanced ABA-sensitivity of PYL4A194T OE plants, since diurnal stomatal movements are linked to ABA concentration via its effect on ion and sugar fluxes (Tallman, 2004).

The lower Gst values of PYL4 and PYL4A194T OE plants suggest that under steady-state conditions, the stomata of PYL4 and PYL4A194T OE plants have reduced aperture compared to non-transformed Col plants. Indeed, direct measurements of stomatal aperture using whole leaf imaging revealed that stomata of both PYL4 and particularly PYL4A194T OE plants were more closed than those of non-transformed Col plants (FIG. 6C). Finally, we also performed water-loss assays of non-transformed Col, PYL4 and PYL4A194T OE lines (FIGS. 6, D and E). Initially, water-loss experiments were done using 15-d-old seedlings grown in a growth chamber, which were excised from Petri dishes and submitted to the drying atmosphere of a laminar flow hood (FIG. 6D). Water-loss kinetics indicated that PYL4A194T OE lines lost less water than non-transformed or PYL4 OE lines (FIG. 6D). Water loss was also measured under greenhouse conditions in plants submitted to drought stress (FIG. 6E). To this end, leaves were detached from plants after 11-d without irrigation, they were subsequently weighted, incubated in de-mineralized water for 3 h and weighed again (FIG. 6E). As a result, we found that PYL4A194T OE lines showed reduced water loss compared to non-transformed Col and PYL4 OE plants. Interestingly, PYL4 OE lines also showed reduced water loss compared to non-transformed plants (FIG. 6E).

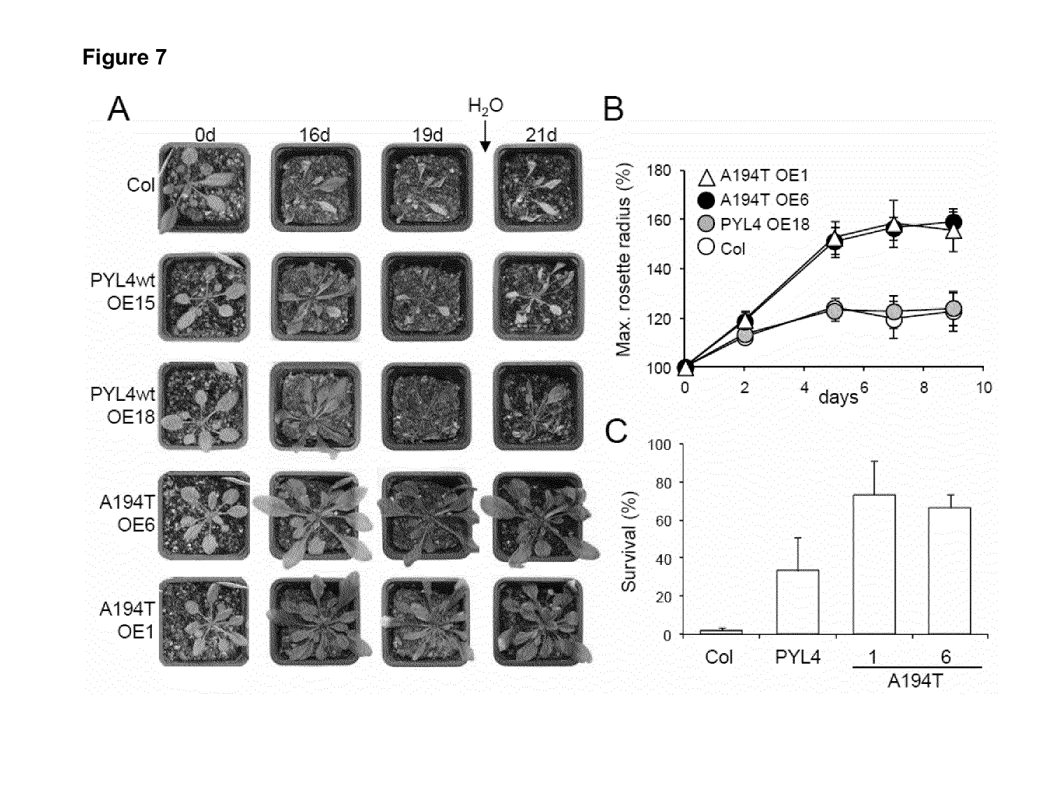

Finally, we performed drought resistance experiments under greenhouse conditions (FIG. 7). Plants were grown in a greenhouse under normal watering conditions for 15 d and then irrigation was stopped. This day was taken as 0-d and average rosette radius did not differ significantly among non-transformed Col, PYL4 OE and PYL4A194T OE plants (FIGS. 7, A and B). However, we found that during the subsequent 5-d plant growth was reduced in non-transformed Col and PYL4 OE plants compared to PYL4A194T OE plants (FIG. 7B). Severe wilting and yellowing of leaves were observed at 16-d in wt, in contrast to PYL4A194T OE lines. Finally, at 19-d watering was resumed and survival of the plants was scored at 23-d. FIG. 7C shows that Col wt plants did not survive after drought stress, whereas around 30% and 60-70% of PYL4 and PYL4A194T OE lines survived, respectively. Finally, since PYL4A194T and PYL4H82RV97A OE lines were hypersensitive to ABA and this hormone is crucial for dehydration tolerance, we tested whether they showed enhanced survival after suffering severe dehydration in Petri dishes. These experiments were done using 15-d-old seedlings by submitting them to dehydration for 12 h in a laminar flow hood, followed by rehydration and scoring survival rate 3-d afterwards. Dehydration experiments revealed enhanced resistance of PYL4A194T and PYL4H82RV97A OE lines compared to PYL4 OE and non-transformed plants. Thus, approximately 40% of PYL4A194T and 25% of PYL4H82RV97A plants survived after 12 h of dehydration followed by rehydration (FIG. 7D).

Transgenic Barley Plants Overexpressing Either PYL4A194T or PYL4H82R V97A show Enhanced Drought Tolerance at the Vegetative Stage

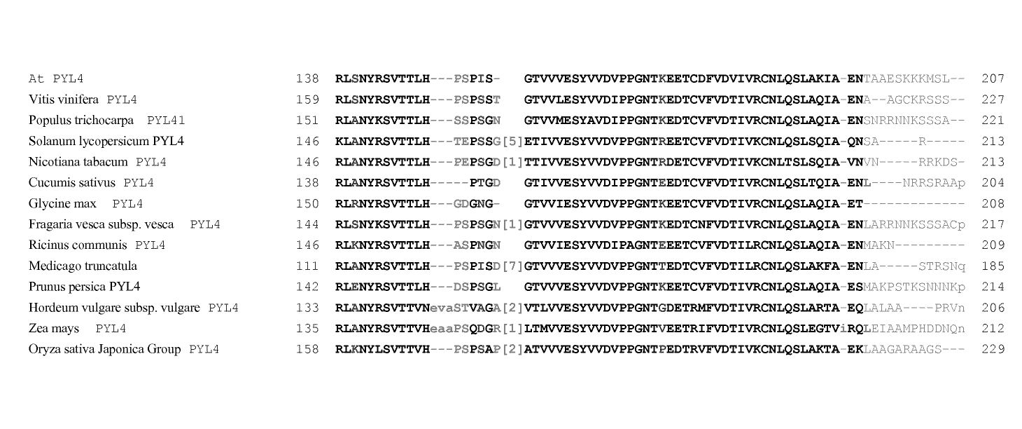

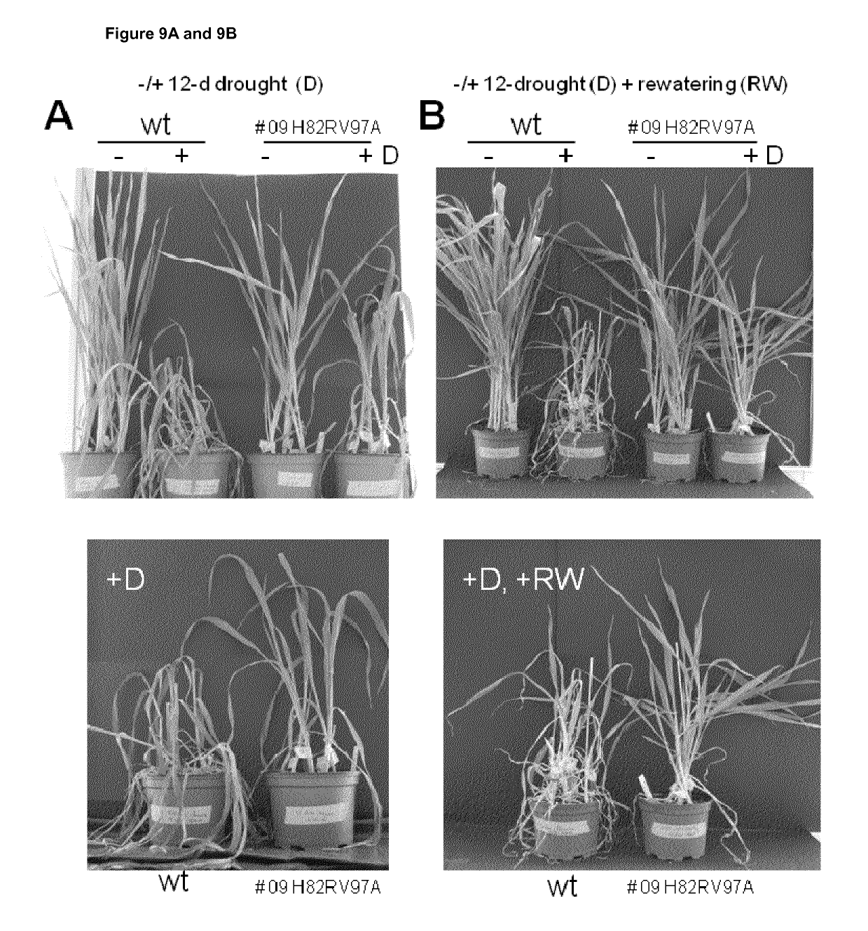

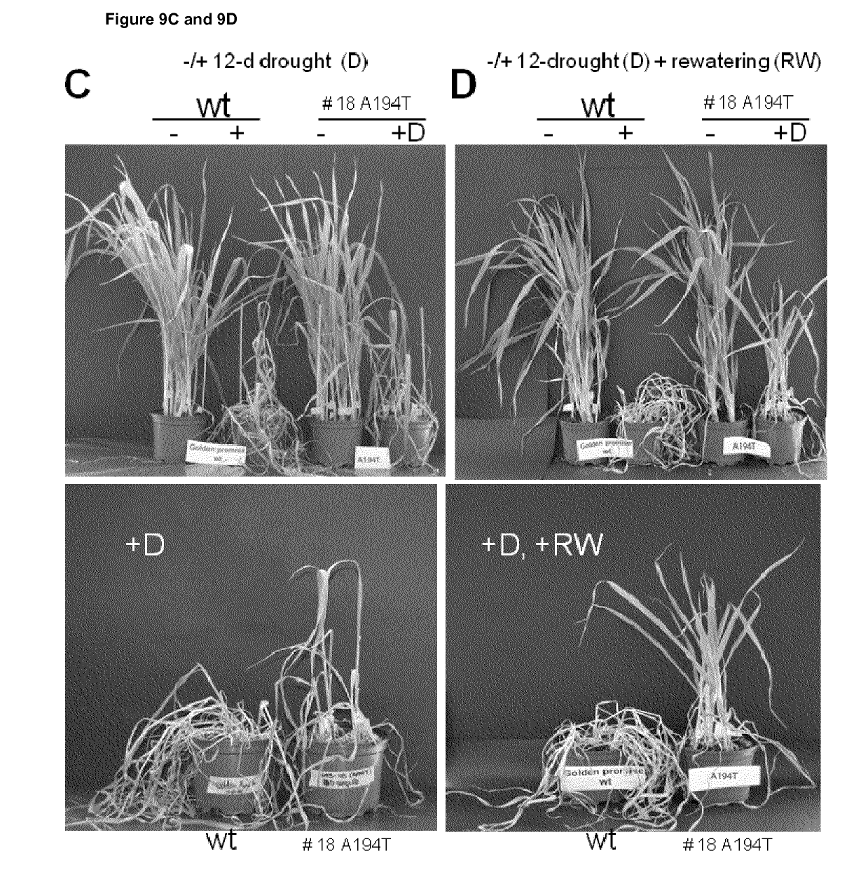

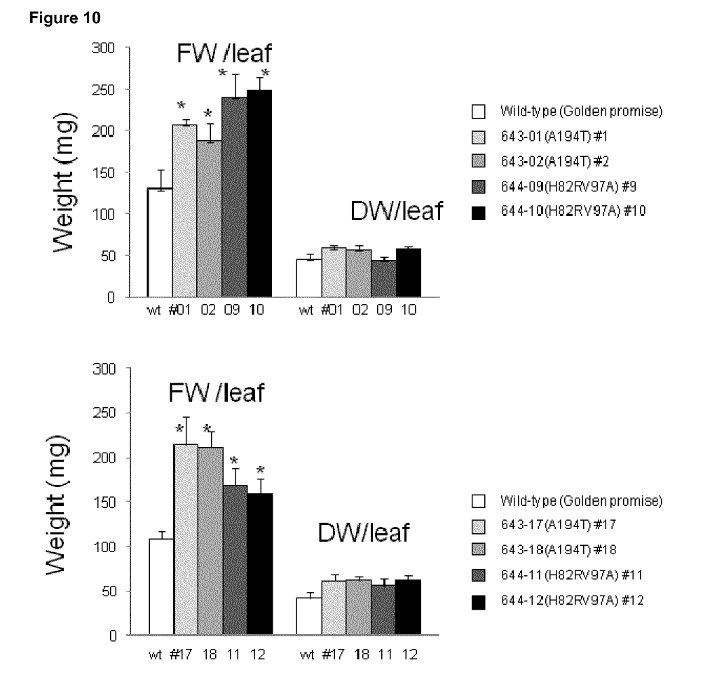

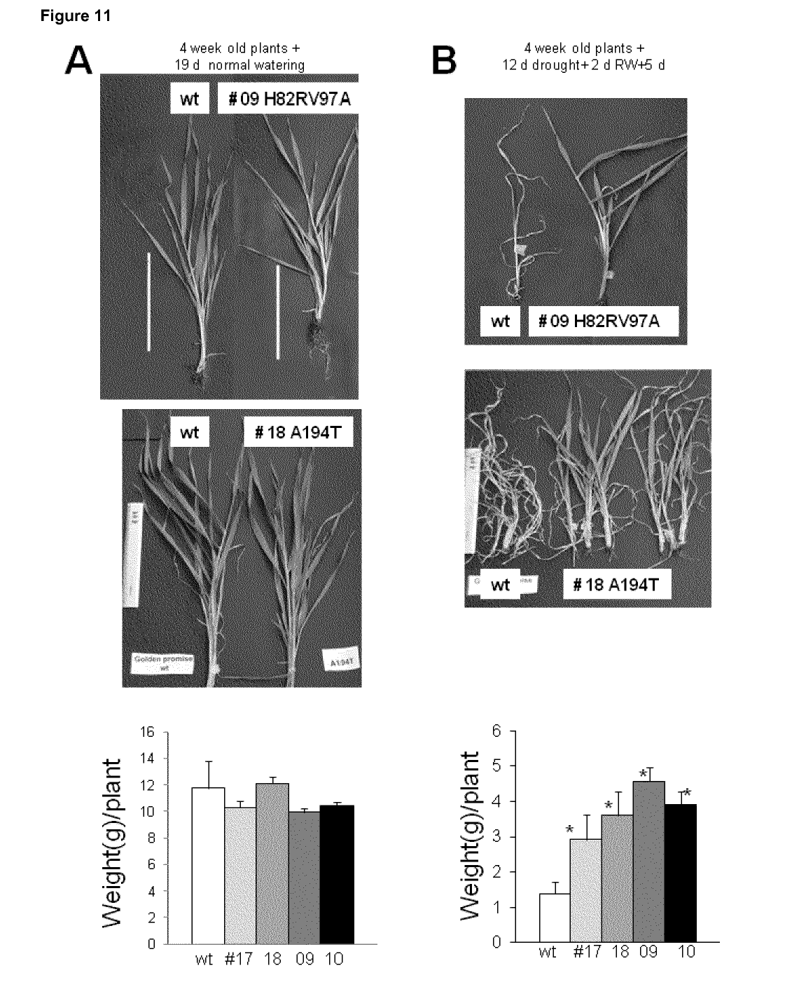

In order to demonstrate the efficacy of the mutant receptors in crop plants, we generated barley (Hordeum vulgare) transgenic plants that over-express mutant versions of Arabidopsis PYL4 receptors (encoded by the SEQ ID NO: 55 and 56). The demonstration of PYL4 technology in barley, in addition to the intrinsic value itself, would be invaluable in pointing the way for other cereal crops of huge agricultural value, such as maize, wheat and rice. Transgenic barley plants were generated (see methods) and they were subjected to drought stress (see methods). As a result, we found that after a 12-d period of drought, transgenic plants overexpressing either PYL4A194T or PYL4H82R V97A showed enhanced drought tolerance compared to non-transformed Golden promise wt plants (FIG. 9, less yellow leaves and higher leaf turgor in transgenic plants). Plant submitted to drought were rewatered for 2-d, then water was withdrawn and photographs were taken after 5-d (FIG. 9). Plants overexpressing either PYL4A194T or PYL4H82R V97A showed enhanced survival and vegetative growth (FIG. 9), higher fresh weight/leaf (FIG. 10) and higher weight/plant (FIG. 11) compared to non-transformed plants

### DISCUSSION

Under non-stress conditions, endogenous levels of ABA play a critical role to regulate stomatal aperture, as revealed by the open stomata phenotype of multiple pyr/pyl mutants, and basal ABA signaling is also required for proper plant growth and development (Barrero et al., 2005; Gonzalez-Guzman et al., 2012; Antoni et al., 2013). On the other hand, plant response to drought is largely dependent on enhanced ABA biosynthesis and signaling in order to regulate both stomatal aperture and gene expression under water stress conditions. Thus, some mutants or transgenic plants showing enhanced response to ABA also display enhanced drought resistance and reduced water consumption (Pei et al., 1998; Hugouvieux et al., 2001; Saez et al., 2006). In this work we describe a novel approach to boost the interaction of PYL4 and PP2CA and to confer drought resistance through genetic engineering of mutated ABA receptors. We generated a PYL4 allele library by error-prone PCR mutagenesis and selected a mutation that enabled ABA-independent interaction of PYL4 with PP2CA. Y2H, in vitro protein-protein interaction and BiFC assays revealed that PYL4A194T showed a distinct pattern of interaction with PP2CA with respect to PYL4 (FIGS. 1 and 3). Thus, both Y2H and pull-down assays indicated that PYL4A194T constitutively interacted with PP2CA in the absence of ABA. BiFC assays showed enhanced interaction of PYL4A194T and PP2CA compared to PYL4 at the endogenous ABA levels present in Agroinfiltrated tobacco cells. This interaction did not affect notably the in vitro phosphatase activity of PP2CA in the absence of ABA (around 20%); however, it might interfere in vivo with PP2CA target recognition by steric hindrance. In vitro phosphatase activity is usually measured using small substrates that might still gain access to the active site of the enzyme, but in vivo activity requires additional contact sites of the phosphatase close to but different from the active site (Soon et al., 2012). For instance, it has been described that an inactive form of PP2CA is able to inhibit OST1 kinase activity simply by forming a complex (Lee et al., 2009). We also found that in the presence of ABA the inhibition of the dephosphorylation of some PP2CA substrates, such as ABI5 and SLAC1, was improved by PYL4A194T compared to PYL4, whereas it did not improve for other substrates, such as ABF2. It is likely that PYL4A194T also displays faster association kinetics with PP2CA than PYL4 in the presence of ABA, particularly at low-intermediate levels of ABA, which might explain the ABA-dependent effects of the mutation. Finally, since the ability of PP2CA to interact with its targets seems to be a requisite for phosphatase function, it is conceivable that the enhanced interaction of PYL4A194T with PP2CA impairs phosphatase activity both under basal and stress conditions.

Activation of ABA receptors by mutational stabilization of the agonist-bound conformation led to ABA-independent inhibition of HAB1, ABI1 and ABI2 (Mosquna et al., 2011). Triple and quadruple mutant combinations were constructed to generate constitutively active (CA) PYR1, PYL2 and PYL9 receptors, which efficiently blocked phosphatase activity in the absence of ABA. As a result, expression of a 35S:GFP-PYL2CA transgene in Arabidopsis seeds activated ABA signaling. However, the existence of a post-transcriptional mechanism that abolished expression of PYL2CA in vegetative tissue precluded further analysis (Mosquna et al., 2011). In contrast, expression of PYL4A194T could be detected in vegetative tissues of 35S:PYL4A194T transgenic plants, which showed hypersensitivity with respect to seed and vegetative responses to ABA. Moreover, 35S:PYL4A194T exhibited enhanced drought resistance compared to non-transformed or 35S:PYL4 OE plants. Particularly interesting features were the partial de-repression of ABA responsive genes, reduced stomatal aperture and transpiration of these lines under basal conditions, which likely contributes to the enhanced drought resistance observed in these plants. Indeed, these plants showed reduced Gst and transpiration under basal conditions as well as reduced water loss when submitted to drought stress (FIG. 6E).

The effect of PYL4A194T appeared to be specific for PP2CA with respect to HAB1, since it did not show a differential effect on HAB1 compared to PYL4. However, at this stage, we cannot exclude that other clade A phosphatases (for instance other members of the PP2CA branch) might also be differentially affected by PYL4A194T. Alignment of clade A PP2Cs reveals two subgroups (the ABM and PP2CA branches) and subtle differences in some regions of the proteins that could affect the interaction with PYR/PYLs (Santiago et al., 2012). Indeed, previous results revealed a certain specificity in the multiple interactions of the 9 clade A PP2Cs and 14 PYR/PYLs (Santiago et al., 2009; Szostkiewicz et al., 2009) and a differential inhibition of PP2CA by PYR/PYLs was recently reported (Antoni et al., 2012). Structural evidence for the PYL4^{A194T}-PP2CA complex is currently not available; however, taking as model other complexes can be observed a clear difference in the length of the α2β4 loop of clade A PP2Cs, which is close to the receptor-phosphatase binding interface. Additionally, the A194 residue is located at the C-terminal helix of PYL4, close to the receptor-phosphatase binding interface. Therefore, the A194T mutation might also indirectly influence the interaction of the C-terminal helix of PYL4 with PP2CA.

In summary, taking into account the phenotype described here for PYL4A194T, the introduction of mutations in PYR/PYL genes that promote ABA-independent interactions with certain PP2Cs might serve as a new tool to ameliorate drought stress. Expression driven by a strong constitutive promoter might lead to some pleiotropic effects that negatively affect growth or yield of crop plants. Such a drawback could be bypassed by introducing stress-inducible or tissue-specific promoters that would drive the expression of the receptor only under stress conditions or in certain tissues. However, expression of either PYL4A194T or PYL4H82R V97A under control of the ubiquitin promoter in transgenic barley plants did not impair vegetative growth under non-stress conditions and on the other hand, it enhanced drought tolerance under stress conditions (FIGS. 9, 10 and 11)

### Material and Methods

### Plant Material and Growth Conditions

Arabidopsis thaliana plants were routinely grown under greenhouse conditions (40-50% relative humidity) in pots containing a 1:3 vermiculite-soil mixture. For plants grown under growth chamber conditions, seeds were surface sterilized by treatment with 70% ethanol for 20 min, followed by commercial bleach (2.5% sodium hypochlorite) containing 0.05% Triton X-100 for 10 min, and finally, four washes with sterile distilled water. Stratification of the seeds was conducted in the dark at 4° C. for 3 days. Seeds were sowed on Murashige-Skoog (MS) plates composed of MS basal salts, 0.1% 2-[N-morpholino]ethanesulfonic acid, 1% sucrose and 1% agar. The pH was adjusted to 5.7 with KOH before autoclaving. Plates were sealed and incubated in a controlled environment growth chamber at 22° C. under a 16 h light, 8 h dark photoperiod at 80-100 μE m-2 sec-1.

Construction of a PYL4 Mutant Library and Analysis of Yeast Two Hybrid Interaction with PP2CA

We conducted error-prone PCR mutagenesis by amplification of the PYL4 open reading frame using the following primers: FPYL4Ncol, 5″-GCAGCAGCCATGGTTGCCG TTCACCGTCCTTCT and RPYL4EcoRIstop: CGCACGAATTCACAGAGACA TCT TCTTCTT, and the following conditions: 2 mM dGTP, dCTP and dTTP, 0.5 mM dATP, 12 mM MgCl2 and Taq polymerase. The PCR product was Ncol-EcoRI doubly digested, cloned into the pGBKT7 vector and DH10B cells were transformed by electroporation. Thus, we generated an allele library in E. coli of approximately 10,000 PYL4 mutant clones. The sequencing of 50 clones revealed on average 1.7 non-silent mutations per clone in the PYL4 sequence (207 amino acids). The library was shuttled to yeast AH109 by co-transformation with pGAD7-PP2CA. Yeast transformants were pooled and clones able to grow in the absence of exogenous ABA in medium lacking histidine and adenine were selected. Yeast plasmids were extracted, sequenced and retransformed in yeast cells to recapitulate the phenotype. Protocols for yeast two hybrid assays were similar to those described previously (Saez et al., 2008).

BiFC Assay in N. benthamiana

Experiments were performed basically as described by Voinnet et al., (2003). The different binary vectors described above where introduced into Agrobacterium tumefaciens C58C1 (pGV2260) (Deblaere et al., 1985) by electroporation and transformed cells were selected in LB plates supplemented with kanamycin (50 μg/ml). Then, they were grown in liquid LB medium to late exponential phase and cells were harvested by centrifugation and resuspended in 10 mM morpholinoethanesulphonic (MES) acid-KOH pH 5.6 containing 10 mM MgCl2 and 150 mM acetosyringone to an OD600 nm of 1. These cells were mixed with an equal volume of Agrobacterium C58C1 (pCH32 35S:p19) expressing the silencing suppressor p19 of tomato bushy stunt virus (Voinnet et al., 2003) so that the final density of Agrobacterium solution was about 1. Bacteria were incubated for 3 h at room temperature and then injected into young fully expanded leaves of 4-week-old N. benthamiana plants. Leaves were examined after 3-4 days under a Leica TCS-SL confocal microscope and laser scanning confocal imaging system. Quantification of fluorescent protein signal was done as described (Gampala et al., 2007) using the National Institutes of Health (NIH) Image software ImageJ v1.37.

Constructs were done in pSPYNE-35S (Walter et al., 2004) as well as gateway vector pYFPC43 (a derivative of pMDC43 where GFP is replaced by YFPC, Belda-Palazon et al., 2012). The coding sequence of At2g38310 (PYL4) was cloned into the pENTR223.1-Sfi entry vector, kindly provided by ABRC (clone G12806). The coding sequence of PYL4A194T was PCR amplified, cloned into the pCR8/GW/TOPO and verified by sequencing. Next, constructs containing PYL4 and PYL4A194T were recombined by LR reaction into pYFPC43 destination vector. The coding sequence of HAB1 and PP2CA was excised from a pCR8/GW/TOPO construct using a double digestion BamHI-StuI and subcloned into BamHI-SmaI doubly digested pSPYNE-35S.

### Protein Expression and Purification

For small scale protein purifications, E. coli BL21 (DE3) cells transformed with the corresponding constructs were grown in 100 ml of LB medium to an OD600 of 0.6-0.8. At this point 1 mM isopropyl-β-D-thiogalactoside (IPTG) was added and the cells were harvested after overnight incubation at 20° C. Pellets were resuspended in lysis buffer (50 mM Tris pH 7.5, 250 mM KCl, 10% Glycerol, 1 mM β-mercaptoethanol) and lysed by sonication with a Branson Sonifier 250. The clear lysate obained after centrifugation was purified by Ni-affinity. A washing step was performed using 50 mM Tris, 250 mM KCl, 20% Glycerol, 30 mM imidazole and 1 mM 3-mercaptoethanol washing buffer, and finally the protein was eluted using 50 mM Tris, 250 mM KCl, 20% Glycerol, 250 mM imidazole and 1 mM 3-mercaptoethanol elution buffer For protein-protein interaction experiments, the pET28a_ΔNPP2CA, pETM11_PYL4 wt and pETM11_PYL4A194T plasmids were transformed into E. coli BL21 (DE3). A total of 8 ml of an overnight culture were sub-cultured into 800 ml fresh 2TY broth (16 g Bacto tryptone, 10 g yeast extract, 5 g NaCl per litre of solution) plus kanamycin (50 μg ml-1). Protein expression was induced with 0.3 mM IPTG and the cells were harvested after overnight incubation at 20° C. Pellets were resuspended in 25 mM TrisHCl pH 8.0, 50 mM NaCl, 50 mM imidazole, 5 mM 3-mercaptoethanol and disrupted by sonication. After centrifugation (40 min, 40000 g) at 277 K, the clear supernatant was filtered (pore diameter 0.45 mm; Millipore Corporation, Bedford, Mass., USA). The 6His-tagged proteins were purified using Ni-NTA Agarose (Qiagen) according to the manufacturer's instructions. The filtered supernatant was mixed with the previously equilibrated beads. After incubation, a washing step with ten volumes of 25 mM TrisHCl pH 8.0, 50 mM NaCl, 20 mM imidazole, 5 mM 3-mercaptoethanol buffer was performed followed by the elution from the Ni2+ resin in a buffer with 500 mM imidazole. Imidazole was removed using a PD-10 column (GE Healthcare) and the His-tag was cleaved using TEV protease.

### Binding Assay of 6His-ΔNPP2CA and PYL4

6His-ΔNPP2CA pellets were resuspended in 25 mM TrisHCl pH 8.0, 150 mM NaCl, 50 mM imidazole, 5 mM 3-mercaptoethanol, 5 mM Mg2+, mixed with 8 mg of either pure non-tagged (through TEV cleavage) PYL4 or PYL4A194T and disrupted by sonication. The crude extracts were treated as described above using His-Trap HP columns from GE Healthcare to the capture step according to the manufacturer's instructions. In all cases, the purified proteins were subjected to a size exclusion chromatography using a Superdex200 10/300 (Amersham Biosciences Limited, UK) to analyze the behavior in a gel filtration of each protein and to isolate the complex. In order to perform pull-down assays, 6His-ΔNPP2CA was purified, next immobilized on Ni-NTA agarose beads (Qiagen) and incubated with either pure non-tagged PYL4 or PYL4A194T. The mix was swirled 30 min at 4° C. and incubated in the absence or presence of 100 μM ABA. After three washes, proteins were eluted by adding 500 mM imidazol and analyzed by SDS-PAGE.

### PP2C and OST1 In Vitro Activity Assays

Phosphatase activity was measured using as a substrate either pNPP or phosphorylated ΔC-ABF2, ΔC-ABI5 and SLAC11-186 proteins. For the pNPP substrate, assays were performed in a 100 μl solution containing 25 mM Tris-HCl pH 7.5, 2 mM MnCl2 and 5 mM pNPP. Assays contained 2 μM phosphatase (PP2CA or HAB1), 4 μM receptor and the indicated concentrations of ABA. Phosphatase activity was recorded with a ViktorX5 reader at 405 nm every 60 seconds over 30 minutes and the activity obtained after 30 minutes is indicated in the graphics. In order to obtain phosphorylated ΔCABF2, ΔC-ABI5 and SLAC11-186 proteins, OST1 phosphorylation assays were done basically as described previously (Dupeux et al., 2011 b). ΔC-ABF2 and SLAC11-186 N-terminal fragments were prepared as described (Antoni et al., 2012; Vahisalu et al., 2010). ΔC-ABI5 recombinant protein (amino acid residues 1-200, containing the C1, C2 and C3 target sites of ABA-activated SnRK2s) was expressed in the pETM11 vector as described above. The reaction mixture containing the OST1 kinase and either ΔCABF2, ΔC-ABI5 or SLAC11-186 recombinant proteins were incubated for 50 min at room temperature in 30 μl of kinase buffer: 20 mM Tris-HCl pH 7.8, 20 mM MgCl2, 2 mM MnCl2, and 3.5 μCi of γ-32ATP (3000 Ci/mmol). Thus, OST1 was autophosphorylated and in turn it phosphorylated ΔC-ABF2, ΔC-ABI5 and SLAC11-186 proteins. Next, they were used as substrates of PP2CA that was preincubated (or not) for 10 with PYL4 or PYL4A194T (1:10 phosphatase:receptor ratio) either in the absence or presence of 30 μM ABA. The reaction was stopped by adding Laemmli buffer and the proteins were separated by SDS-PAGE using an 8% acrylamide gel and transferred to an Immobilon-P membrane (Millipore). Radioactivity was detected and quantified using a Phosphorimage system (FLA5100, Fujifilm). After scanning, the same membrane was used for Ponceau staining. The data presented are averages of at least three independent experiments.

### Generation of Transgenic Lines

PYL4 or PYL4 mutants were cloned into pCR8/GW/TOPO entry vector (Invitrogen) and recombined by LR reaction into the gateway compatible ALLIGATOR2 vector (Bensmihen et al., 2004). This construct drives expression of PYL4 under control of the 35S CaMV promoter and introduces a triple HA epitope at the N-terminus of the protein. Selection of transgenic lines is based on the visualization of GFP in seeds, whose expression is driven by the specific seed promoter At2S3. The ALLIGATOR2-35S:3HA-PYL4 or mutant constructs were transferred to Agrobacterium tumefaciens C58C1 (pGV2260) (Deblaere et al., 1985) by electroporation and used to transform Columbia wild type plants by the floral dip method. T1 transgenic seeds were selected based on GFP visualization and sowed in soil to obtain the T2 generation. At least three independent transgenic lines were generated for each construct. Homozygous T3 progeny was used for further studies and expression of HA-tagged protein in 21-d-old seedlings was verified by immunoblot analysis using anti-HA-peroxidase (Roche).

### Seed Germination and Seedling Establishment Assays.

After surface sterilization of the seeds, stratification was conducted in the dark at 4° C. for 3 d. Approximately 100 seeds of each genotype were sowed on MS plates supplemented with different ABA concentrations per experiment. To score seed germination, radical emergence was analyzed at 72 h after sowing. Seedling establishment was scored as the percentage of seeds that developed green expanded cotyledons and the first pair of true leaves at 7-d.

### Root and Shoot Growth Assays.

Seedlings were grown on vertically oriented MS plates for 4 to 5 days. Afterwards, 20 plants were transferred to new MS plates lacking or supplemented with the indicated concentrations of ABA. The plates were scanned on a flatbed scanner after 10-d to produce image files suitable for quantitative analysis of root growth using the NIH software ImageJ v1.37. As an indicator of shoot growth, the maximum rosette radius was measured.

### RNA Analyses

ABA treatment, RNA extraction and quantitative RT-PCR amplifications were performed as previously described (Saez et al., 2004).

### Whole-Rosette Stomatal Conductance and Transpiration Measurements

The Arabidopsis whole-rosette gas exchange measurement device, plant growth practice and custom written program to calculate transpiration and Gst for water vapour have been described previously (Kollist et al. 2007; Vahisalu et al., 2008). For gas-exchange experiments, 25-28-d-old plants (rosette area 6-18 cm2) were used. Until measurements, plants were grown in growth chambers (AR-66LX and AR-22L, Percival Scientific, IA, USA) at 12/12 photoperiod, 23/18° C. temperature, air relative humidity of 70-80% and 150 μmol m-2 s-1 light. During gas exchange measurements, temperature, air relatively humidity, photoperiod and light in the cuvettes were kept as similar as possible to the

values in growth chambers. Photographs of plants were taken before and after the experiment and rosette leaf area was calculated using the NIH software ImageJ 1.37v. Leaf area values for the intermediary experimental period were calculated using linear regression between starting and final leaf area.

### Water-Loss and Stomatal Aperture Assays.

2-3 weeks-old seedlings grown in MS plates were used for water-loss assays. Four seedlings per genotype with similar growth, three independent experiments, were submitted to the drying atmosphere of a flow laminar hood. Kinetic analysis of waterloss was performed and represented as the percentage of initial fresh weight loss at each scored time point. Stomatal aperture measurements were done in leaves of 5-week-old plants grown under greenhouse conditions using whole leaf imaging (Chitrakar and Melotto, 2010). Staining of whole leaves with propidium iodide was conducted and the aperture of 30-40 stomata (ratio width/length, two independent experiments) was measured using a Leica TCS-SL confocal microscope.

### Drought Stress

Plants grown under greenhouse conditions (10 individuals per experiment, three independent experiments) were grown under normal watering conditions for 15 days and then subjected to drought stress by stopping irrigation during 20 days. Next, watering was resumed and survival rate was calculated after 3 days by counting the percentage of plants that had more than four green leaves. Photographs were taken at the start of the experiment (day 0), after 16 and 19 days of drought, and 3 days after rewatering. Shoot-growth and water-loss were measured as follows. Quantification of shoot-growth was performed at 2, 5, 7 and 9 d after stopping irrigation (day 0) by measuring the maximum rosette radius of the plants. Water-loss measurements were done in two leaves from each plant that were detached 11 d after the start of the experiment, next weighted, incubated in de-mineralized water for 3 h and weighed again. The difference in weight was considered as water loss and related to the initial fresh weight (μl H₂O/g FW).

### Dehydration Treatment

2-weeks-old seedlings grown in MS plates were used for these experiments. Twenty seedlings per genotype (two independent experiments) were submitted to the drying atmosphere of a flow laminar hood for 12 hours (25° C.±1° C., 25%±2% relative humidity), then rehydrated with 25 ml of water. Survival percentage was scored 3 days after rehydration by counting the percentage of plants that had at least four green leaves.

### Accession Numbers

The Arabidopsis Genome Initiative locus identifiers for PYL4 and PP2CA are At2g38310 and At3g11410, respectively.

### Expression in Barley

Barley Plant Material. Construction of the Vector and Transgenic Lines.

In order to demonstrate the efficacy of the mutant receptors in crop plants, we generated barley (Hordeum vulgare) transgenic plants that over-express mutant versions of Arabidopsis PYL4 receptors (encoded by the SEQ ID NO: 55 and 56). Transgenic barley (cv. Golden Promise) expressing either Arabidopsis PYL4^{A194T} or PYL4^{H82R V97A} open reading frame driven by the Ubiquitin promoter from the pBract214 vector was generated via Agrobacterium-mediated transformation (Bartlett et al. 2008).

The nucleotide sequence encoding either PYL4^{A194T} or PYL4^{H82R V97A} open reading frame was recombined by LR reaction from pCR8/GW/TOPO entry vector into the Gateway compatible pBract214 destination vector. The sequence introduced in barley is the Arabidopsis open reading frame carrying the indicated mutations. Also, the second codon (of the sequence as shown in SEQ ID NO; 2) was modified to GTT to get a Ncol site to facilitate cloning).

Immature embryos were inoculated with Agrobacterium strain AGL1 containing the over-expression vector pBract214 into which the genes of interest had been cloned. pBract214 also contains the hygromycin resistance gene to allow selection of transgenic tissues and plants. Following co-cultivation for 3 days, immature embryos were transferred to selective callus induction medium containing hygromycin to allow the selection of transformed tissue and timentin to remove Agrobacterium. After a total of 6 weeks callus induction, callus were moved to a transition medium in low light and then 2 weeks later to regeneration medium under full light. Regenerated plants were transferred to rooting tubes when shoots reached 2-3 cm in length. Plants with strong roots in hygromycin containing medium were then established in soil and grown to maturity under controlled environment conditions in order to obtain T1 seed progeny.

### Growth Conditions and Drought Stress Treatment

Barley plants (cv. Golden Promise) were routinely grown under greenhouse conditions (40-50% relative humidity, 23-24° C.) in pots containing a 1:3 vermiculite-soil mixture. Pots were grouped in trays where water was maintained approximately 0.2-1 cm above the bottom of the tray. For drought (D) stress experiments, four-week-old plants were watered with tap water for 12-d (minus D, water maintained 0.2-1 cm above the bottom of the tray) or were submitted to drought for 12-d (plus D, water withdrawal). One flag leaf per plant (10 individual plants for each genetic background, minus and plus D treatment) was weighed in order to obtain its fresh weight and dried for 16 h at 70° C. and weighed again to obtain its dry weight. After the 12 day drought period, plants that had been submitted to drought were re-watered (RW, water 1 cm above the bottom of the tray for 2-d, then water withdrawal) and weight per plant (destructive measurement to obtain above ground biomass) was obtained after 5-d (plus D, plus RW). The results obtained are described in FIGS. 9, 10 and 11.

### LIST OF REFERENCES

Antoni R, Gonzalez-Guzman M, Rodriguez L, Rodrigues A, Pizzio G A, Rodriguez P L (2012) Selective Inhibition of Clade A Phosphatases Type 2C by PYR/PYL/RCAR Abscisic Acid Receptors. Plant Physiol 158: 970-980
Antoni R, Gonzalez-Guzman M, Rodriguez L, Peirats-Llobet M, Pizzio G A, Fernandez M A, De Winne N, De Jaeger G, Dietrich D, Bennett M J, Rodriguez P L (2013) PYRABACTIN RESISTANCE1-LIKE8 μlays an important role for the regulation of abscisic acid signaling in root. Plant Physiol 161: 931-941
Barrero J M, Piqueras P, Gonzalez-Guzman M, Serrano R, Rodriguez P L, Ponce M R, Micol J L (2005) A mutational analysis of the ABA1 gene of Arabidopsis thaliana highlights the involvement of ABA in vegetative development. J Exp Bot 56: 2071-2083
Bartlett J G, Alves S C, Smedley M, Snape J W, Harwood W A (2008) High-throughput Agrobacterium-mediated barley transformation. Plant Methods 4: 22
Belda-Palazon B, Ruiz L, Marti E, Tarraga S, Tiburcio A F, Culianez F, Farras R, Carrasco P, Ferrando A (2012) Aminopropyltransferases involved in polyamine biosynthesis localize preferentially in the nucleus of plant cells. PLoS One 7: e46907
Bensmihen S, To A, Lambert G, Kroj T, Giraudat J, Parcy F (2004) Analysis of an activated ABI5 allele using a new selection method for transgenic Arabidopsis seeds. FEBS Lett 561: 127-131
Chitrakar R, Melotto M (2010) Assessing stomatal response to live bacterial cells using whole leaf imaging. J Vis Exp
Cutler S R, Rodriguez P L, Finkelstein R R, Abrams S R (2010) Abscisic acid: emergence of a core signaling network. Annu Rev Plant Biol 61: 651-679
Deblaere R, Bytebier B, De Greve H, Deboeck F, Schell J, Van Montagu M, Leemans J (1985) Efficient octopine Ti plasmid-derived vectors for Agrobacterium-mediated gene transfer to plants. Nucleic Acids Res 13: 4777-4788
Dupeux F, Santiago J, Betz K, Twycross J, Park S Y, Rodriguez L, Gonzalez-Guzman M, Jensen M R, Krasnogor N, Blackledge M, Holdsworth M, Cutler S R, Rodriguez P L, Marquez J A (2011a) A thermodynamic switch modulates abscisic acid receptor sensitivity. EMBO J 30: 4171-4184
Dupeux F, Antoni R, Betz K, Santiago J, Gonzalez-Guzman M, Rodriguez L, Rubio S, Park S Y, Cutler S R, Rodriguez P L, Marquez J A (2011 b) Modulation of Abscisic Acid Signaling in Vivo by an Engineered Receptor-Insensitive Protein Phosphatase Type 2C Allele. Plant Physiol 156: 106-116
Gampala S S, Kim T W, He J X, Tang W, Deng Z, Bai M Y, Guan S, Lalonde S, Sun Y, Gendron J M, Chen H, Shibagaki N, Ferl R J, Ehrhardt D, Chong K, Burlingame A L, Wang Z Y (2007) An essential role for 14-3-3 proteins in brassinosteroid signal transduction in Arabidopsis. Dev Cell 13: 177-189
Gonzalez-Guzman M, Pizzio G A, Antoni R, Vera-Sirera F, Merilo E, Bassel G W, Fernandez M A, Holdsworth M J, Perez-Amador M A, Kollist H, Rodriguez P L (2012) Arabidopsis PYR/PYL/RCAR Receptors Play a Major Role in Quantitative Regulation of Stomatal Aperture and Transcriptional Response to Abscisic Acid. Plant Cell 24: 2483-2496
Hao Q, Yin P, Li W, Wang L, Yan C, Lin Z, Wu J Z, Wang J, Yan S F, Yan N (2011) The Molecular Basis of ABA-Independent Inhibition of PP2Cs by a Subclass of PYL Proteins. Mol Cell 42: 662-672
Hugouvieux V, Kwak J M, Schroeder J I (2001) An mRNA cap binding protein, ABH1, modulates early abscisic acid signal transduction in Arabidopsis. Cell 106: 477-487
Iuchi S, Kobayashi M, Taji T, Naramoto M, Seki M, Kato T, Tabata S, Kakubari Y, Yamaguchi-Shinozaki K, Shinozaki K (2001) Regulation of drought tolerance by gene manipulation of 9-cisepoxycarotenoid dioxygenase, a key enzyme in abscisic acid biosynthesis in Arabidopsis. Plant J 27: 325-333
Jakab G, Ton J, Flors V, Zimmerli L, Metraux J P, Mauch-Mani B (2005) Enhancing Arabidopsis salt and drought stress tolerance by chemical priming for its abscisic acid responses. Plant Physiol 139: 267-274
Kollist T, Moldau H, Rasulov B, Oja V, Ramma H, Huve K, Jaspers P, Kangasjarvi J, Kollist H (2007) A novel device detects a rapid ozone-induce transient stomatal closure in intact Arabidopsis and its absence in abi2 mutant. Physiol Plant 129: 796-803
Kuhn J M, Boisson-Dernier A, Dizon M B, Maktabi M H, Schroeder J I (2006) The protein phosphatase AtPP2C A negatively regulates abscisic acid signal transduction in Arabidopsis, and effects of abh1 on AtPP2C A mRNA. Plant Physiol 140: 127-139
Lackman P, Gonzalez-Guzman M, Tilleman S, Carqueijeiro I, Perez A C, Moses T, Seo M, Kanno Y, Hakkinen S T, Van Montagu M C, Thevelein J M, Maaheimo H, Oksman-Caldentey K M, Rodriguez P L, Rischer H, Goossens A (2011) Jasmonate signaling involves the abscisic acid receptor PYL4 to regulate metabolic reprogramming in Arabidopsis and tobacco. Proc Natl Acad Sci USA 108: 5891-5896
Lee S C, Lan W, Buchanan B B, Luan S (2009) A protein kinase-phosphatase pair interacts with an ion channel to regulate ABA signaling in plant guard cells. Proc Natl Acad Sci USA 106: 21419-21424
Ma Y, Szostkiewicz I, Korte A, Moes D, Yang Y, Christmann A, Grill E (2009) Regulators of PP2C Phosphatase Activity Function as Abscisic Acid Sensors. Science 324: 1064-1068
Mosquna A, Peterson F C, Park S Y, Lozano-Juste J, Volkman B F, Cutler S R (2011) Potent and selective activation of abscisic acid receptors in vivo by mutational stabilization of their agonist-bound conformation. Proc Natl Acad Sci USA 108: 20838-20843
Pei Z M, Ghassemian M, Kwak C M, McCourt P, Schroeder J I (1998) Role of farnesyltransferase in ABA regulation of guard cell anion channels and plant water loss. Science 282: 287-290
Qin X, Zeevaart J A (2002) Overexpression of a 9-cis-epoxycarotenoid dioxygenase gene in Nicotiana plumbaginifolia increases abscisic acid and phaseic acid levels and enhances drought tolerance. Plant Physiol 128: 544-551
Ran et al. Genome engineering using the CRISPR-Cas9 system nature protocols, VOL. 8 NO. 11, 2281-2308, 2013
Saavedra X, Modrego A, Rodriguez D, Gonzalez-Garcia M P, Sanz L, Nicolas G, Lorenzo O (2010) The nuclear interactor PYL8/RCAR3 of Fagus sylvatica FsPP2C1 is a positive regulator of abscisic acid signaling in seeds and stress. Plant Physiol 152: 133-150
Saez A, Apostolova N, Gonzalez-Guzman M, Gonzalez-Garcia M P, Nicolas C, Lorenzo O, Rodriguez P L (2004) Gain-of-function and loss-of-function phenotypes of the protein phosphatase 2C HAB1 reveal its role as a negative regulator of abscisic acid signalling. Plant J 37: 354-369
Saez A, Robert N, Maktabi M H, Schroeder J I, Serrano R, Rodriguez P L (2006) Enhancement of abscisic acid sensitivity and reduction of water consumption in Arabidopsis by combined inactivation of the protein phosphatases type 2C ABI1 and HAB1. Plant Physiol 141: 1389-1399
Saez A, Rodrigues A, Santiago J, Rubio S, Rodriguez P L (2008) HAB1-SWI3B interaction reveals a link between abscisic acid signaling and putative SWI/SNF chromatin-remodeling complexes in Arabidopsis. Plant Cell 20: 2972-2988
Santiago J, Rodrigues A, Saez A, Rubio S, Antoni R, Dupeux F, Park S Y, Marquez J A, Cutler S R, Rodriguez P L (2009a) Modulation of drought resistance by the abscisic acid receptor PYL5 through inhibition of clade A PP2Cs. Plant J 60: 575-588
Santiago J, Dupeux F, Round A, Antoni R, Park S Y, Jamin M, Cutler S R, Rodriguez P L, Marquez J A (2009b) The abscisic acid receptor PYR1 in complex with abscisic acid. Nature 462: 665-668
Santiago J, Dupeux F, Betz K, Antoni R, Gonzalez-Guzman M, Rodrigues L, Marquez J, Rodriguez P L (2011) Structural insights into PYR/PYL/RCAR ABA receptors and PP2Cs. Plant Science 182: 3-11
Soon F F, Ng L M, Zhou X E, West G M, Kovach A, Tan M H, Suino-Powell K M, He Y, Xu Y, Chalmers M J, Brunzelle J S, Zhang H, Yang H, Jiang H, Li J, Yong E L, Cutler S, Zhu J K, Griffin P R, Melcher K, Xu H E (2012) Molecular mimicry regulates ABA signaling by SnRK2 kinases and PP2C phosphatases. Science 335: 85-88
Tallman G (2004) Are diurnal patterns of stomatal movement the result of alternating metabolism of endogenous guard cell ABA and accumulation of ABA delivered to the apoplast around guard cells by transpiration? J Exp Bot 55: 1963-1976
Vahisalu T, Kollist H, Wang Y F, Nishimura N, Chan W Y, Valerio G, Lamminmaki A, Brosche M, Moldau H, Desikan R, Schroeder J I, Kangasjarvi J (2008) SLAC1 is required for plant guard cell Stype anion channel function in stomatal signalling. Nature 452: 487-491
Vahisalu T, Puzorjova I, Brosche M, Valk E, Lepiku M, Moldau H, Pechter P, Wang Y S, Lindgren O, Salojarvi J, Loog M, Kangasjarvi J, Kollist H (2010) Ozone-triggered rapid stomatal response involves the production of reactive oxygen species, and is controlled by SLAC1 and OST1. Plant J 62: 442-453
Voinnet O, Rivas S, Mestre P, Baulcombe D (2003) An enhanced transient expression system in plants based on suppression of gene silencing by the p19 protein of tomato bushy stunt virus. Plant J 33: 949-956
Walter M, Chaban C, Schutze K, Batistic O, Weckermann K, Nake C, Blazevic D, Grefen C, Schumacher K, Oecking C, Harter K, Kudla J (2004) Visualization of protein interactions in living plant cells using bimolecular fluorescence complementation. Plant J 40: 428-438
Yoshida T, Nishimura N, Kitahata N, Kuromori T, Ito T, Asami T, Shinozaki K, Hirayama T (2006) ABA-hypersensitive germination3 encodes a protein phosphatase 2C (AtPP2CA) that strongly regulates abscisic acid signaling during germination among Arabidopsis protein phosphatase 2Cs. Plant Physiol 140: 115-126
Zhang X, Zhang Q, Xin Q, Yu L, Wang Z, Wu W, Jiang L, Wang G, Tian W, Deng Z, Wang Y, Liu Z, Long J, Gong Z, Chen Z (2012) Complex structures of the abscisic acid receptor PYL3/RCAR13 reveal a unique regulatory mechanism. Structure 20: 780-790

Sequence listing

SEQ ID NO: 1: AtPYL4 nucleic acid sequence (genomic; coding
sequence including residues from the 5′ and 3′ UTR)
aaatcgaaag gcacagccca acttttcgca agtcgctgta aagtttgatt tgcttctttt
tatatacaca catacttctc ctccatacac tttcctcttc aatcctcagt tttttttcta
agccctaata ccatctcaaa gaagagatca agatttgaaa tcaagaagac accattactc
agatcaacat gcttgccgtt caccgtcctt cttccgccgt atcagacgga gattccgttc
agattccgat gatgatcgcg tcgtttcaaa aacgttttcc ttctctctca cgcgactcca
cggccgctcg ttttcacaca cacgaggttg gtcctaatca gtgttgctcc gccgttattc
aagagatctc cgctccaatc tccaccgttt ggtccgtcgt acgccgcttt gataacccac
aagcttacaa acactttctc aaaagctgta gcgtcatcgg cggagacggc gataacgttg
gtagcctccg tcaagtccac gtcgtctctg gtctccccgc cgctagctcc accgagagac
tcgatatcct cgacgacgaa cgccacgtca tcagcttcag cgttgttggt ggtgaccacc
ggctctctaa ctaccgatcc gtaacgaccc ttcacccttc tccgatctcc gggaccgtcg
ttgtcgagtc ttacgtcgtt gatgttcctc caggcaacac aaaggaagag acttgtgact
tcgttgacgt tatcgtacga tgcaatcttc aatctcttgc gaaaatagcc gagaatactg
cggctgagag caagaagaag atgtctctgt gatgagtctt tgtcgttgtc gggtagtttc
gttagatccg acgtcgtttt ctagattttt agccgtcgtg tgatctatgt tttttcggct
tatgtgtgaa aaaaaagtta cattagtgaa ttaatctctc atgcatatca taatccttct
tttaattttt gtattttaca tatcccataa agaaccgatt tggatagccc tattccggct
ttcaccaccc aaagataata atattcaaac t
SEG ID NO: 2: AtPYL4 nucleic acid sequence (cDNA; coding sequence)
atgcttgccg ttcaccgtcc ttcttccgcc gtatcagacg gagattccgt tcagattccg
atgatgatcg cgtcgtttca aaaacgtttt ccttctctct cacgcgactc cacggccgct
cgttttcaca cacacgaggt tggtcctaat cagtgttgct ccgccgttat tcaagagatc
tccgctccaa tctccaccgt ttggtccgtc gtacgccgct ttgataaccc acaagcttac
aaacactttc tcaaaagctg tagcgtcatc ggcggagacg gcgataacgt tggtagcctc
cgtcaagtcc acgtcgtctc tggtctcccc gccgctagct ccaccgagag actcgatatc
ctcgacgacg aacgccacgt catcagcttc agcgttgttg gtggtgacca ccggctctct
aactaccgat ccgtaacgac ccttcaccct tctccgatct ccgggaccgt cgttgtcgag
tcttacgtcg ttgatgttcc tccaggcaac acaaaggaag agacttgtga cttcgttgac
gttatcgtac gatgcaatct tcaatctctt gcgaaaatag ccgagaatac tgcggctgag
agcaagaaga agatgtctct gtga
SEQ ID NO: 3: AtPYL4 protein sequence (>lcl|AT2G38310.1)
MLAVHRPSSAVSDGDSVQIPMMIASFQKRFPSLSRDSTAARFHTHEVGPNQCCSAVIQEISAPIS
TVWSVVRRFDNPQAYKHFLKSCSVIGGDGDNVGSLRQVHVVSGLPAASSTERLDILDDERHVISF
SVVGGDHRLSNYRSVTTLHPSPISGTVVVESYVVDVPPGNTKEETCDFVDVIVRCNLQSLAKIAE
NTAAESKKKMSL
SEQ ID NO: 4: AtPYL4 mRNA
aaatcgaaag gcacagccca acttttcgca agtcgctgta aagtttgatt tgcttctttt
tatatacaca catacttctc ctccatacac tttcctcttc aatcctcagt tttttttcta
agccctaata ccatctcaaa gaagagatca agatttgaaa tcaagaagac accattactc
agatcaacat gcttgccgtt caccgtcctt cttccgccgt atcagacgga gattccgttc
agattccgat gatgatcgcg tcgtttcaaa aacgttttcc ttctctctca cgcgactcca
cggccgctcg ttttcacaca cacgaggttg gtcctaatca gtgttgctcc gccgttattc
aagagatctc cgctccaatc tccaccgttt ggtccgtcgt acgccgcttt gataacccac
aagcttacaa acactttctc aaaagctgta gcgtcatcgg cggagacggc gataacgttg
gtagcctccg tcaagtccac gtcgtctctg gtctccccgc cgctagctcc accgagagac
tcgatatcct cgacgacgaa cgccacgtca tcagcttcag cgttgttggt ggtgaccacc
ggctctctaa ctaccgatcc gtaacgaccc ttcacccttc tccgatctcc gggaccgtcg
ttgtcgagtc ttacgtcgtt gatgttcctc caggcaacac aaaggaagag acttgtgact
tcgttgacgt tatcgtacga tgcaatcttc aatctcttgc gaaaatagcc gagaatactg
cggctgagag caagaagaag atgtctctgt gatgagtctt tgtcgttgtc gggtagtttc
gttagatccg acgtcgtttt ctagattttt agccgtcgtg tgatctatgt tttttcggct
tatgtgtgaa aaaaaagtta cattagtgaa ttaatctctc atgcatatca taatccttct
tttaattttt gtattttaca tatcccataa agaaccgatt tggatagccc tattccggct
ttcaccaccc aaagataata atattcaaac tgaaagaatg tggttgtgtt gtccgctaat
taaaagtgtg attttcaagt ttaatt
SEQ ID NO: 5: PYL4 Vitis vinifera nucleic acid sequence
(genomic; coding sequence including residues from the 5′ UTR)
XP_002264158.1
ccacttagcc ctattcctca aaccccacaa gcccctccac ctcctcttcc tcccatcaat
cttaatacca aaagtgaaat aaaaatgccc tcaaaccctc caaaatcctc gcttgtggtt
catagaatca acagtcctaa tagcattacc actgccacca ccgcttctgc cgccgcgaat
aaccataata cttcaaccat gcctcctcac aagcaagttc agaagcggtc tcctctaacg
agcgccaccc aggtccccga cgctgtgtca cgccaccaca cccacgttgt cggccccaat
caatgctgct ccgccgtcgt ccagcaaatc gccgcccctg tctccaccgt ctggtccgtc
gtccgccgct tcgacaatcc acaggcctac aagcacttcg tcaagagttg ccacgtcgtt
gttggcgatg gagatgtcgg cactctccgc gaggtccacg tcatctccgg cctccccgcg
gccaacagca ccgagcgcct cgaaatcctc gacgacgagc gccatgtcct cagcttcagc
gtgatcggtg gtgaccaccg cctctccaat taccgatcgg tcaccactct ccacccctcc
ccctccagca ccggcaccgt ggtcctggaa tcctatgtcg ttgacatacc cccaggaaac
accaaagaag acacgtgtgt ttttgtcgac accatcgttc ggtgcaacct gcaatccctt
gctcagatcg cggagaacgc cgccggctgc aagaggtcat cgtcatga
SEQ ID NO: 6 PYL4 Vitis vinifera nucleic acid sequence
(cDNA; coding sequence)
atgccctcaa accctccaaa atcctcgctt gtggttcata gaatcaacag tcctaatagc
attaccactg ccaccaccgc ttctgccgcc gcgaataacc ataatacttc aaccatgcct
cctcacaagc aagttcagaa gcggtctcct ctaacgagcg ccacccaggt ccccgacgct
gtgtcacgcc accacaccca cgttgtcggc cccaatcaat gctgctccgc cgtcgtccag
caaatcgccg cccctgtctc caccgtctgg tccgtcgtcc gccgcttcga caatccacag
gcctacaagc acttcgtcaa gagttgccac gtcgttgttg gcgatggaga tgtcggcact
ctccgcgagg tccacgtcat ctccggcctc cccgcggcca acagcaccga gcgcctcgaa
atcctcgacg acgagcgcca tgtcctcagc ttcagcgtga tcggtggtga ccaccgcctc
tccaattacc gatcggtcac cactctccac ccctccccct ccagcaccgg caccgtggtc
ctggaatcct atgtcgttga cataccccca ggaaacacca aagaagacac gtgtgttttt
gtcgacacca tcgttcggtg caacctgcaa tcccttgctc agatcgcgga gaacgccgcc
ggctgcaaga ggtcatcgtc atga
SEQ ID NO: 7: PYL4 Vitis vinifera protein sequence
mpsnppkssl vvhrinspns ittattasaa annhntstmp phkqvqkrsp ltsatqvpda
vsrhhthvvg pnqccsavvq qiaapvstvw svvrrfdnpq aykhfvksch vvvgdgdvgt
lrevhvisgl paansterle ildderhvls fsviggdhrl snyrsvttlh pspsstgtvv
lesyvvdipp gntkedtcvf vdtivrcnlq slaqiaenaa gckrsss
SEQ ID NO: 8: PYL4 Populus trichocarpa nucleic acid sequence
(genomic/cDNA sequence; coding sequence); XP_002323024.1
atgcctgcta atcctccgag gtcatccctt ttaatccata gaatcaacaa caccacaagt
aacacaaccc taaacaccac caacacaacc accgcaactt catgccaaaa acggtggtcc
cctctaccat gcgacgccac cccagttccg gagaccgtct cacgctacca cacccacgct
gtaggcccca accagtgctg ctctgcggtg gtacaacaga tcgctgcccc aatctccacc
gtatggtctg tcgttcgccg gttcgacaac ccacaagctt ataaacattt cgtcaagagc
tgccacgtta tcctcggaga cggtgacgtg ggcactctcc gtgaaatcca cgttatctcg
ggcctcccag ctgctcacag caccgaacgc ctcgagatcc tcgacgatga acggcatgtc
atcagtttta gtgttgttgg tggggaccat aggcttgcga attacaagtc ggttacaact
ctccattcgt ctccctcagg gaacggcacc gtcgtcatgg agtcttacgc ggtcgatatt
cctcctggaa atactaaaga ggacacgtgt gtgttcgtgg ataccatagt tcggtgcaac
ctgcagtcac tggcacagat cgccgagaac tcgaatagac gcaataataa atcatcatca
gcgtga
SEQ ID NO: 9: PYL4 Populus trichocarpa protein sequence
mpanpprssl lihrinntts nttlnttntt tatscqkrws plpcdatpvp etvsryhtha
vgpnqccsav vggiaapist vwsvvrrfdn pqaykhfvks chvilgdgdv gtlreihvis
glpaahster leildderhv isfsvvggdh rlanyksvtt lhsspsgngt vvmesyavdi
ppgntkedtc vfvdtivrcn lqslagiaen snrrnnksss a
SEQ ID NO: 10: PYL4 Solanum lycopersicum nucleic acid sequence
(genomic: coding sequence including residues from the
5′ and 3′ UTR); XP_004249671.1
ggctcccact agccccacta ttttgtcaac ctcacgtgaa gggtgaaccc ccatctcttt
tttttttttt aaatactttt ctcaacttca accaactatt tcttaattag ctctaatttc
tacactgtat atatatatac aacttaatcg caccaataag aacacaagag agaagaaatc
atcactactt tttcattttc tctcaatttt caaatcagca acaaaaatgc ctccaagttc
ttcagattca tctgttttac tccaacgaat tagctctaac aatactcatg attttgctta
caagcaatct catcatcagt tacagagacg catgccgatt ccctgttcga cggaagtacc
tgattctgtt tcccggtacc atactcacac tgtaagtcct gaccagtgct gctccgccgt
gatccagcgg atctcagctc ctgtctccac cgtatggtcg gtggtccgcc gatttgacaa
tccgcaggcg tataagcatt ttgttaaaag ctgtcatgtt gttgttggcg atggtgacgt
cggtacttta cgggaagtcc gtgtgatttc cggtctccct gctgctagta gcacggagag
gctagagatc ctcgacgacg aacgccacgt catcagtttt agcgttgtcg gtggggacca
caagttagcc aattaccgat ctgtgactac gcttcacacg gaaccgagct ccggcaatga
agcggcggcg gagacgatcg ttgtggaatc gtacgtcgtt gatgtaccgc ctggtaatac
tagagaagag acgtgcgttt ttgtggacac aatcgtgaaa tgcaaccttc aatcgttatc
tcagatcgcg cagaattcag ccagatgaaa atcttcgtca attttgaata tgtattttgg
atcgatcaac ttgctctaac gcttcacttt tcgattatat tcgtgttgaa aatttcaaaa
atatacattt ttgaagtatt taatacgtat atattcatat tttgaagagt tcgaacaggt
cgcggatctg aattaatgaa gacaactaat cgatgatgct tgtggtgatg atctgatgat
atcatatgcg atgcatgatt gtttcgttgc acctttatta ggtgtgtgtt aatctccttg
tcatcacttg ttgaagataa gttcttgatc aaattaatct ttgtactttg cttaactaat
ttcatactct ataattttgt ctatattgtt ttctttatcg atttcgttat catggtatga
gacagattca tatatcttaa ttttaatatg ataatttgat ttgttttg
SEQ ID NO: 11: PYL4 Solanum lycopersicum nucleic acid sequence
(cDNA: coding sequence)
atgcctccaa gttcttcaga ttcatctgtt ttactccaac gaattagctc taacaatact
catgattttg cttacaagca atctcatcat cagttacaga gacgcatgcc gattccctgt
tcgacggaag tacctgattc tgtttcccgg taccatactc acactgtaag tcctgaccag
tgctgctccg ccgtgatcca gcggatctca gctcctgtct ccaccgtatg gtcggtggtc
cgccgatttg acaatccgca ggcgtataag cattttgtta aaagctgtca tgttgttgtt
ggcgatggtg acgtcggtac tttacgggaa gtccgtgtga tttccggtct ccctgctgct
agtagcacgg agaggctaga gatcctcgac gacgaacgcc acgtcatcag ttttagcgtt
gtcggtgggg accacaagtt agccaattac cgatctgtga ctacgcttca cacggaaccg
agctccggca atgaagcggc ggcggagacg atcgttgtgg aatcgtacgt cgttgatgta
ccgcctggta atactagaga agagacgtgc gtttttgtgg acacaatcgt gaaatgcaac
cttcaatcgt tatctcagat cgcgcagaat tcagccagat ga
SEQ ID NO: 12: PYL4 Solanum lycopersicum protein sequence
mppsssdssv llqrissnnt hdfaykqshh qlqrrmpipc stevpdsysr yhthtvspdq
ccsaviqris apvstvwsvv rrfdnpqayk hfvkschvvv gdgdvgtlre vrvisglpaa
ssterleild derhvisfsv vggdhklany rsvttlhtep ssgneaaaet ivvesyvvdv
ppgntreetc vfvdtivkcn lqslsqiaqn sar
SEQ ID NO: 13: PYL4 Nicotiana tabacum nucleic acid sequence
(genomic/cDNA sequence; coding sequence); CAI84653.1
ATGCCTCCTAGTTCTCCAGATTCATCTGTTTTACTCCAAAGAATAAGCTCCAACACTACT
CCTGATTTTGCCTGTAAACAATCTCAGCAATTACAAAGGCGTACTATGCCGATACCTTGT
ACGACACAAGTCCCAGATTCCGTTGTCCGATTCCATACTCACCCAGTGGGTCCCAACCAG
TGCTGCTCCGCCGTGATCCAGCGGATTTCCGCCCCCGTCTCCACCGTATGGTCAGTTGTC
CGCCGCTTCGACAACCCGCAAGCATACAAGCATTTCGTCAAGAGCTGCCACGTCATCGTA
GGGGATGGTGACGTCGGCACTCTCCGCGAGGTTCGCGTGATCTCAGGCCTTCCAGCTGCG
TCCAGCACGGAAAGACTCGAGATCCTCGACGACGAGCGCCATGTCATCAGCTTCAGCGTA
GTCGGCGGGGACCACCGACTCGCGAATTACCGTTCCGTCACCACCCTCCACCCGGAACCG
TCTGGTGACGGGACGACCATCGTCGTTGAATCCTACGTTGTTGATGTACCACCTGGTAAT
ACTAGAGATGAAACGTGTGTTTTCGTCGATACCATCGTCAAATGCAACCTCACATCGTTA
TCGCAGATCGCAGTAAACGTGAACAGAAGAAAAGATTCTTGA
SEQ ID NO: 14: PYL4 Nicotiana tabacum protein sequence
mppsspdssv llqrissntt pdfackqsqq lqrrtmpipc ttqvpdsvvr fhthpvgpnq
ccsaviqris apvstvwsvv rrfdnpqayk hfvkschviv gdgdvgtlre vrvisglpaa
ssterleild derhvisfsv vggdhrlany rsvttlhpep sgdgttivve syvvdvppgn
trdetcvfvd tivkcnitsl sqiavnvnrr kds
SEQ ID NO: 15: PYL4 Cucumis sativus nucleic acid sequence,
XP_004148626.1
aagtgaaaag ctgccatcgc cgcggctccg atttcagcta tataaatcag gcagcaaagg
agaagaaaag agaaacccca cttacacgga aaatgcctcc taatccccct aaatcatccg
ccgcctccca tcgcataacc cactcctcca ccgtgccgga gtttttcaag cgccagattc
aaacacgcgc taccgctgtt cctgacgcgg tggcgcgtta ccacaaccac gctgtttcca
tgaaccagtg ctgctccgcc gtcgttcaag agatcgatgc cccggtctcc accgtgtggt
ccgtcgtccg ccgcttcgac aacccgcaag cgtacaagca cttcgtcaag agctgtgacg
tcatcgtcgg tgacggcaac gttggaagcc tccgtgaagt tcgcgtcatt tccggcctcc
cggcagccaa cagcaccgag cggctggaga ttctcgacga cgaacgtcat attattagct
tcagtgtcgt cggcggcgaa caccgactcg ctaactaccg gtccgttacc actctccacc
caaccggcga cggcaccatc gtagtggaat cgtacgtcgt cgacattcct ccgggaaaca
ccgaggagga cacgtgtgtg ttcgtcgaca ccatcgtccg atgcaacctt cagtcactga
ctcagatcgc tgagaacctg aatcgccgga gccgagcagc gccgccgtga
SEQ ID NO: 16: PYL4 Cucumis sativus protein sequence
mppnppkssa ashrithsst vpeffkrqiq tratavpdav aryhnhaysm nqccsavvqe
idapvstvws vvrrfdnpqa ykhfvkscdv ivgdgnvgsl revrvisglp aansterlei
ldderhiisf svvggehrla nyrsvttlhp tgdgtivves yvvdippgnt eedtcvfvdt
ivrcnlqslt qiaenlnrrs raapp
SEQ ID NO: 17: PYL4 Glycine max nucleic acid sequence (genomic;
coding sequence including residues from the 5′ and 3′ UTR);
XP_003519420.1
ctttaacata aaatcacaaa acctatatct ttattattat tattattatt attattatta
ttattgctat tattatcaca tgaactagaa atgacttctc ttcaattcca ccgattcaac
ccagcaaccg atacatccac cgccatcgca aacggcgtca actgtccgaa gccaccgtca
acgctccgtt tattggcgaa agtaagcctt agcgtgccgg agacggttgc tcggcaccac
gcgcacccgg tggggcccaa ccagtgctgc tccgtcgtga tccaggcgat cgatgcaccg
gtctccgccg tctggccggt ggtgcggcgc ttcgacaacc cgcaggccta caagcacttc
gtgaagagct gccacgtggt cgccgcagca ggcggcggcg aggacggcat tcgcgtcggc
gcgctccggg aggtacgcgt ggtctccggg ctccccgccg tgtcgagcac cgagcggctc
gagatcctcg acgacgagcg ccacgtcatg agcttcagcg tcgtcggcgg cgaccaccgc
ctaaggaact accgctccgt cacgacgctc cacggcgacg gcaacggagg gacagttgta
atcgagtcgt acgtggttga cgtaccgccc ggtaacacta aagaagagac ttgcgttttc
gttgacacaa tcgtacggtg caatttgcag tccctggctc agatcgctga aacctgaaaa
tatggcaagc caacaacact caacatttta ttttctccgt tagttttttt ggttattgcc
ttgggtgttt ttttgtttcg aatacgggtt cgggttcgaa attgatgatt acagacacca
ttaggatttg tttttggagg acttatcaag gtcagacgac aatggttgaa atgaagaatg
tgacatataa tatatgatat atgattcatg tatatataca tataatatat gtagttcttt
ctatctacct ctcttttatt ttttccattc taatagctgt tgagtctgat cgaatctatt
gttcttgtat ccgtggtcaa tttatcatta attcatcttg tttgtgagtc tgtaatgtag
tagagaatgt ttctatttga attaatacca tacatatgtt gttattagct ataggaccaa
attagtttac aacttgtcct g
SEQ ID NO: 18: PYL4 Glycine max nucleic acid sequence
(cDNA; coding sequence)
atgacttctc ttcaattcca ccgattcaac ccagcaaccg atacatccac cgccatcgca
aacggcgtca actgtccgaa gccaccgtca acgctccgtt tattggcgaa agtaagcctt
agcgtgccgg agacggttgc tcggcaccac gcgcacccgg tggggcccaa ccagtgctgc
tccgtcgtga tccaggcgat cgatgcaccg gtctccgccg tctggccggt ggtgcggcgc
ttcgacaacc cgcaggccta caagcacttc gtgaagagct gccacgtggt cgccgcagca
ggcggcggcg aggacggcat tcgcgtcggc gcgctccggg aggtacgcgt ggtctccggg
ctccccgccg tgtcgagcac cgagcggctc gagatcctcg acgacgagcg ccacgtcatg
agcttcagcg tcgtcggcgg cgaccaccgc ctaaggaact accgctccgt cacgacgctc
cacggcgacg gcaacggagg gacagttgta atcgagtcgt acgtggttga cgtaccgccc
ggtaacacta aagaagagac ttgcgttttc gttgacacaa tcgtacggtg caatttgcag
tccctggctc agatcgctga aacctga
SEQ ID NO: 19: PYL4 Glycine max protein sequence
mtslqfhrfn patdtstaia ngvncpkpps tlrllakvsl svpetvarhh ahpvgpnqcc
svvigaidap vsavwpvvrr fdnpqaykhf vkschvvaaa gggedgirvg alrevrvvsg
1payssterl eildderhvm sfsvvggdhr lrnyrsvttl hgdgnggtvv iesyvvdvpp
gntkeetcvf vdtivrcnlq slaqiaet
SEQ ID NO: 20: PYL4 Fragaria vesca subsp. vesca nucleic acid
sequence; XP_004302617.1
gcttgttttt tcctcctttt ttgctgatca aggaccaact tgccttctat tttatcatcc
cacatgaact ctcccaagat ttaatattca catttcctcc cctttgaaat atacaaccac
cccatcttct ctcttcttca aacaatcccc aaagctctgc tagcttcaag aaactaagct
cagagatcat cctctaatgc ctcccaaccc acccaagtct tctgtattgt gtcaccgcct
caacaccgcc cccaacacct cgtctaacaa ccagaagaac aacatgatga tcaagaagca
gcagcagcag cgcgccccga tcccagaagc ggtggcgcgt taccacacgc atggagtcgg
ccctaacaag tgctgctccg ccgtgacgca ggagatcgct gctcccgtct ccacagtctg
gtccgtcgtc cgccgcttcg ataatcccca agcctacaag cacttcgtga aaagctgcca
cgttatcgtc ggcgacggcg acgttggtac cctccgcgag gttcaggtca tctcggggct
tccagccaac aacagcactg agaggctgga cgtgctggac gacgagagcc atgtcatcag
cttcagcatg gtcggcggcg accacaggct gtcaaactac aagtccgtga cgacgcttca
cccgtcacct tcgggtaacg gaggcacggt ggttgtggag tcttacgtgg tggacgtgcc
gccggggaac actaaggaag acacgtgtaa cttcgtggac accatcgtcc ggtgcaacct
gcagtcgctg gctcagatcg cagagaatct agcgagacgt aacaacaagt cgtcttcggc
gtgccccaag taataatatg tttatttatt tttaattatt atgatcggag tattattatg
gtgatggtta ttagtaagat aacatcttaa attaggtagc aagagtaatt agcttgtggt
ggtgttctgg gttttatgta gtctattagg tttcgtcgtc gaacatcaat gtctgcgaga
gttgtctgac tccgagtccg agtccggtct gtgtttgccg ttggagggta cggcgtcgtt
ttgtatccga tgaaacagtc acagctgcaa gttttatgat gtaatatata tctagtagtg
cctta
SEQ ID NO: 21: PYL4 Fragaria vesca subsp. vesca protein sequence
mppnppkssv lchrintapn tssnnqknnm mikkqqqqra pipeavaryh thgvgpnkcc
savtqeiaap vstvwsvvrr fdnpqaykhf vkschvivgd gdvgtlrevq visglpanns
terldvldde shvisfsmvg gdhrlsnyks vttlhpspsg nggtvvvesy vvdvppgntk
edtcnfvdti vrcnlqslaq iaenlarrnn ksssacpk
SEQ ID NO: 22: PYL4 Ricinus communis nucleic acid sequence;
XP_002520792.1
gtatgatgca tgataagctc atcttcttca tcatctttcc cccaaatatt tctctacaat
tttctctaac aaaagcctca aactaatcca acttgacaca ttaaccattt tcaagaacaa
acctctcttc gtttcaatct ctattcatat atatatatat atatatattt ttacaaatcc
tgccatttat tacgcatgaa tctttaaccc taacttaaat catatcaaga aactttagcc
aactcaaaat tatgcctgct gcttccctac agctccaaat acccaacact gccaccacca
ccacaaccac cacctcggcc gccttgtctt gctacaagca ttcttggcag ccgccagtgc
ctctttcttg ggatgcagcc gtgcctgact atgtttcttg ctaccataca cgctccgttg
gtccagatca gtgttgctcc gccgtgttca agatcataaa tgcacctgtt tccactgttt
ggtcggtggt tcgccgtttc gataacccac aagcttataa acactttgtc aagagctgtc
atttaatcaa cggtgacggg gatgtaggca cgcttaggga ggttcacgtg gtgtcaggat
tacctgctga atcaagtact gaaagactgg agattctcga tgatgagcaa catgttataa
gctttagcat gatcggcggc gatcaccggc taaagaatta tcggtcagtg acgactcttc
atgcttctcc aaatggaaat ggtacggttg ttattgaatc atatgtagtt gatataccag
caggaaatac tgaggaggaa acctgtgttt ttgttgatac aattttaagg tgcaacttgc
aatccttggc tcagatagct gagaacatgg ccaagaatta gcaaaatcat catcatcatc
atcatcttga tttgtttaca aacgggtttt tcacaactaa aattttttgg tttttgattc
gggttcggat taattcgatc atctgaattg tcaatgcctg aattgggaat ttgtagatcc
atcagctagc agttcatgaa ggtgtttata gtattttgat gctatatatg tcaacttttg
ttttccttca agacattgct ccctcattta gcatacaatt atagtgagat tttatgtttt
acataatgtc ttcttgtatt tatttatttt ctttcttctt ttttgttaat tagttcaagt
tatttttctc ctctttgttt ttgttctaaa aggagagagg ctcttaattt tgtgccttct
tgtatatatt gtgtctagtt ccgcctctct tctttcttta ttatatgtat tttcttttgg
agaaaagaat tatattattt tatttttgta attatctctt ataatattat a
SEQ ID NO: 23: PYL4 Ricinus communis protein sequence
mpaaslqlqi pntatttttt tsaalscykh swqppvplsw daavpdyvsc yhtrsvgpdq
ccsavfkiin apvstvwsvv rrfdnpqayk hfvkschlin gdgdvgtlre vhvvsglpae
ssterleild deqhvisfsm iggdhrlkny rsvttlhasp ngngtvvies yvvdipagnt
eeetcvfvdt ilrcnlqsla qiaenmakn
SEQ ID NO: 24: PYL4 Medicago truncatula nucleic acid sequence
(genomic; coding sequence including residues from the 5′ UTR);
XP_003623366.1
gtccttcctc ctttgtcctt tcaaaaaacc tccacttatt taacttctct cctttctttt
ccaaagacac caaaaccatc ctttcattca ttcacattct caaacctcaa aataaaaaaa
aacatcatat caagtatcaa caacatcatc aaatcaagca ctcgagatca cagactaatc
ttgatcgtca gagtgtttgc aggcacatca tctaaaacac ttgatcgtcg gtcgcagtgt
ttgcaggtac accatctgag cacaagttgt tcttgaataa caaaatatgt taccaaaccc
aacaaccacc gtccccgacg ccatcgcccg ctaccacacc cacgcagtct cccccaacca
gtgttgctcc gccgtcatcc aacatatcgc cgcacccgtc tccaccgtct ggtccgtcgt
ccgtcgcttc gacaatccac aagcctacaa acacttcgtc aaaagctgcc atgtcatcct
cggagacggc aacgtcggca ctctccgtga agtccgtgtc atctccggcc tccccgccgc
cgtcagcacc gaacgtctcg aagtcctaga cgatgaacgt catgtcatca gcttcagcat
gatcggtggc gatcaccgtc ttgctaacta ccgttctgtc accactctcc acccttctcc
gatctccgat gaagatggca accaccgctc cggcacggtg gttgttgagt cctacgttgt
tgatgttcca ccgggaaaca ccactgaaga tacatgtgtc tttgttgata ctattcttcg
gtgcaacctt caatctctcg cgaaatttgc tgagaatttg gcttcaacga gatcaaatca
acgataa
SEQ ID NO: 25: PYL4 Medicago truncatula nucleic acid sequence
(cDNA; coding sequence)
atgttaccaa acccaacaac caccgtcccc gacgccatcg cccgctacca cacccacgca
gtctccccca accagtgttg ctccgccgtc atccaacata tcgccgcacc cgtctccacc
gtctggtccg tcgtccgtcg cttcgacaat ccacaagcct acaaacactt cgtcaaaagc
tgccatgtca tcctcggaga cggcaacgtc ggcactctcc gtgaagtccg tgtcatctcc
ggcctccccg ccgccgtcag caccgaacgt ctcgaagtcc tagacgatga acgtcatgtc
atcagcttca gcatgatcgg tggcgatcac cgtcttgcta actaccgttc tgtcaccact
ctccaccctt ctccgatctc cgatgaagat ggcaaccacc gctccggcac ggtggttgtt
gagtcctacg ttgttgatgt tccaccggga aacaccactg aagatacatg tgtctttgtt
gatactattc ttcggtgcaa ccttcaatct ctcgcgaaat ttgctgagaa tttggcttca
acgagatcaa atcaacgata a
SEQ ID NO: 26: PYL4 Medicago truncatula protein sequence
mlpnptttvp daiaryhtha vspnqccsav iqhiaapvst vwsvvrrfdn pqaykhfvks
chvilgdgnv gtlrevrvis glpaayster levldderhv isfsmiggdh rlanyrsvtt
lhpspisded gnhrsgtvvv esyvvdvppg nttedtcvfv dtilrcnlqs lakfaenlas
trsnqr
SEQ ID NO: 27: Prunus persica nucleotide sequence
(genomic/cDNA; coding sequence)
atgccttcct cactgcaatt ccaaagaccc atcaataact ctcctaattt ttatcccaca
aacacattaa tccaccacaa gcaattccaa tcccacgccg cggccgcagc tgtagcggag
gacttcacgc gcctcaacgc tcacgtgatg tcgcccaacc ccaaccagtg ctgctccgcg
gttgtgcagt ccatcgaggc ccccgtcccg accgtgtggt cggtggtgcg gcgcttcgac
aacccacagg cctacaagca cttcctcaag agctgccagg tcatcgacgg gaccggcgac
gtgggcacgc tccggaaggt ccacgtggtg tcgggcctcc ccgcggggtc cagcacggag
cgcctcgaga tcctcgacga cgagcgacac gtcctgagct tcagtgttgt gggtggggac
caccggctcg agaactaccg atccgtcacc acgctccacg actcgccgag tggactcggg
accgtcgtgg tggagtcgta cgtggtggac gttccgccgg gaaacaccaa ggaggagacg
tgcgtgttcg tcgacaccat cgtgcgctgc aacttgcagt cgctggctca gatcgcggag
agcatggcca aaccatccac caagagtaac aacaacaagc cctcatga
SEQ ID NO: 28: PYL4 Prunus persica protein sequence; EMJ20866.1
mpsslqfqrp innspnfypt ntlihhkqfq shaaaaavae dftrinahvm spnpnqccsa
vvqsieapvp tvwsvvrrfd npqaykhflk scqvidgtgd vgtlrkvhvv sglpagsste
rleildderh vlsfsvvggd hrlenyrsvt tlhdspsglg tvvvesyvvd vppgntkeet
cvfvdtivrc nlqslaqiae smakpstksn nnkps
SEQ ID NO: 29: PYL4 Hordeum vulgare subsp. vulgare nucleic acid
sequence (genomic; coding sequence including residues from the
5′ and 3′ UTR); BAJ93794.1
gagctaatcc taagttccca acccacccac tctcctaaaa tttcttcttc acagcgataa
agctcagaag ctcgagccgc ccgcggcttg tctacaatgc cgtacgcagc tgcacggccg
tcgccccagc agcacagccg gatcagcgcc gcctgtaagg cgttggtggc gcagggtgca
gcggtgccgg gcgaggtggc gcggcaccac gagcacgcgg ccggcgcggg gcagtgctgc
tcggccgtgg tgcaggcgat cgcggcgccc gtggaggcgg tgtggtcggt cgtgcggcgc
ttcgaccggc cgcaggcgta caagcgcttc atcaagagct gccgcctggt ggacggcgac
ggtggcgcgg tgggatcggt gcgggaggtg cgcgtcgtct ccggtctgcc tggcaccagc
agccgcgagc ggctcgagat cctggacgac gagcggcgcg tgctcagctt ccggatcgtc
ggcggcgagc accgcctcgc caactaccgg tccgtcacca ccgtgaacga ggtggcgtcg
acggtggccg gggcgccgcg ggtgaccctg gtggtcgagt cttatgtggt ggacgtgccg
ccggggaaca cgggggacga gacgcgcatg ttcgtggaca ccatcgtgcg gtgcaacctc
cagtcgctcg cgcgcacggc ggagcaactc gcgctggcag cgccgcgcgt gaactgatgc
ccgtcatgca ctccggcaga ccagctgaaa ggtaccaccg tagacccgta ccacgggcgc
aaggtcgagg ttctggaatc tggagtgaca tgatttacac ccatgcatta gccctagtgt
catgtgaaga aagattggct ttgtgcacct ggctgttttg tgtaaatcat ctcatcgtgt
taacttagta gttgaattaa tgtgcagtac tgattggatt
SEQ ID NO: 30: PYL4 Hordeum vulgare subsp. vulgare nucleic acid
sequence (cDNA; coding sequence)
atgccgtacg cagctgcacg gccgtcgccc cagcagcaca gccggatcag cgccgcctgt
aaggcgttgg tggcgcaggg tgcagcggtg ccgggcgagg tggcgcggca ccacgagcac
gcggccggcg cggggcagtg ctgctcggcc gtggtgcagg cgatcgcggc gcccgtggag
gcggtgtggt cggtcgtgcg gcgcttcgac cggccgcagg cgtacaagcg cttcatcaag
agctgccgcc tggtggacgg cgacggtggc gcggtgggat cggtgcggga ggtgcgcgtc
gtctccggtc tgcctggcac cagcagccgc gagcggctcg agatcctgga cgacgagcgg
cgcgtgctca gcttccggat cgtcggcggc gagcaccgcc tcgccaacta ccggtccgtc
accaccgtga acgaggtggc gtcgacggtg gccggggcgc cgcgggtgac cctggtggtc
gagtcttatg tggtggacgt gccgccgggg aacacggggg acgagacgcg catgttcgtg
gacaccatcg tgcggtgcaa cctccagtcg ctcgcgcgca cggcggagca actcgcgctg
gcagcgccgc gcgtgaac
SEQ ID NO: 31: PYL4 Hordeum vulgare subsp. vulgare
protein sequence
mpyaaarpsp qqhsrisaac kalvaqgaav pgevarhheh aagagqccsa vvqaiaapve
avwsvvrrfd rpqaykrfik scrlvdgdgg avgsvrevrv vsglpgtssr erleildder
rvlsfrivgg ehrlanyrsv ttvnevastv agaprvtlvv esyvvdvppg ntgdetrmfv
dtivrcnlqs lartaeqlal aaprvn
SEQ ID NO: 32: PYL4 Zea mays nucleic acid sequence (genomic);
ACG26321.1
ctttccacac ataatgagca tacctacaag ccatattccc atccacattc atcccccaac
acccactcgt ccacccaccg ttttccttgt cttcactttc ctcacgcaat tcacccaccc
cctcaccatt tcccccatcg accattgctg ccacatacca aacccacttc ctgtggtctt
cggtgtcttc cccgccgccg ccgctgccgt cgccgtcgtg catcacactg tccctcttgc
tgtgatatct atgagagaga gaaacagctc gatcgatcaa gaacaccaac gaggctctag
ctccagatcg acaatgccgt tcgcagcctc aaggacgtca cagcagcagc acagccgtgt
ggccaccaac gggagggccg tggcggtgtg cgcgggtcac gcgggcgtgc ccgacgaggt
ggcgcggcac cacgagcacg ctgtggcagc ggggcaatgc tgcgccgcca tggtgcagtc
catcgcagcg ccggtggacg cggtgtggtc gctggtgcgt cgcttcgacc agccgcagcg
gtacaagcgc ttcatcagga gctgccacct cgtggacggc gacggcgccg aggtggggtc
cgtgcgggag ctcctgctcg tgtccgggct gcccgccgag agcagccgcg agcggcttga
gatccgggac gacgagcggc gggtgatcag cttccgggtc ctgggcggcg accaccgcct
ggccaactac cgctccgtga ccaccgtgca cgaggcggcg ccgtcgcagg acgggcgccc
gctcaccatg gtcgtcgagt cctacgtggt ggacgtgccg ccggggaaca ccgtcgagga
gacgcgcatc ttcgtggaca ccatcgttcg gtgcaacctc cagtccctcg agggcacggt
catcaggcag ctggagatcg cggcaatgcc gcacgacgac aaccagaact gattgccgtc
gggccggccg gctgcacacc gtcatcgcag ttgggctgcc ggtttatttg gacgaggctt
catcgaccat ttatgatata ttttctttcg tcgttggtac tgtatactct cgcgaaaata
agaagttggt actgtgaact gcttaaagtt tgaggttgtt tgtcttccct ttcttaaact
ctgccaataa tgaagtattg taagttacag catcaataat attaagacca gtaaagtagt
atagttaaaa aaaaaaaaaa aaaaaaaa
SEQ ID NO: 33 PYL4 Zea mays nucleic acid sequence (cDNA; coding
sequence, GenBank: BT067140.1, ZM_BFb0206N02 mRNA)

| | |
|---|---|
| 1 | gttgagtgga aaaccatata atctgacacc ttcgccacct ctgtctcatc accttttgtt |
| 61 | gttgcctcta ccattagaga ccatacccct accttagtag ctatcagatc tgtttccccc |
| 121 | gacccactca aaatattgaa ccactacttc caatccctaa acctaatgac acctgagcgc |
| 181 | tacccatcat aggatcgcca tcatccattg aataaaatag aagagagaaa caactcgatc |
| 241 | gatcgatcaa gaagaccggc gaggctctag ctccagaccg acaatgccgt acgcagccac |
| 301 | aaggacgtcg ccgcagcagc acagccgcgt ggccagcaac gggagggccg tggcggcgtg |
| 361 | cgcaggccac gcgggcgtgc cggacgaggt ggcgcggcac cacgagcacg cggtggccgc |
| 421 | ggggcagtgc tgctctgtga tggtgcagtc catcgcggcg ccggcggacg cggtgtggtc |
| 481 | gctggtgcgc cgcttcgacc agccgcaggg gtacaagcgc ttcatcagga gctgccacct |
| 541 | ggtggacggc gacggcgtcg aggtggggtc cgtgcgggag ctcctggtcg tgtccgggct |
| 601 | gcccgccgag aacagccgcg agcggcttga gatccgggac gacgagcggc gggtgatcag |
| 661 | cttccggatc ctgggcggcg accaccgcct cgccaactac cgctccgtga ccaccgtgca |
| 721 | cgaggcggcc tcagagggcg ggccgctcac catggtcgtc gagtcctatg tggtggacgt |
| 781 | gccgccgggg aacaccgtcg aggagacgcg catcttcgtg gacaccatcg ttcggtgcaa |
| 841 | cctccaatcc ctcgaggaca cggttatcag gcagcaggcg atggcggcac cggcagcgcc |
| 901 | gcacaacgac cacaaccaca gctgatcgcc gccgggcccg gccggctgga caccctcgca |
| 961 | gtgggggctg ccggtttctt cggaggcttc atctgccatt tatgatgttt tctttcgccg |
| 1021 | ttggtattct atagtttcac caataagaag aataggtact gtgaactttt taaaggttga |
| 1081 | ggttgttcgt cttccctttc ttaaactcta aaaacgacaa taaagtattg tatgatacac |
| 1141 | caccaataat attaagacca gtaaagtagt atagttctaa tatatatgtg taagtttgtg |
| 1201 | tatagat |

SEQ ID NO: 34: PYL4 Zea mays protein sequence, Gen Bank: ACN34037.1

| | |
|---|---|
| 1 | mpyaatrtsp qqhsrvasng ravaacagha gvpdevarhh ehavaagqcc svmvqslaap |
| 61 | adavwslvrr fdqpqgykrf irschlvdgd gvevgsvrel lvvsglpaen srerleirdd |
| 121 | errvisfril ggdhrlanyr svttvheaas eggpltmvve syvvdvppgn tveetrifvd |
| 181 | tivronlqsl edtvirqqam aapaaphndh nhs |

SEQ ID NO: 35: PYL4 Oryza sativa Japonica Group nucleic acid
sequence (genomic; coding sequence including residues from
the 5′ and 3′ UTR)
caacctcctc atcgccgcat agaaattcca ccgaacacat agcgagcaag aagaggagga
ggaggagaag cgaaggagac aaggaaacag cgcgacatgc cgtgcatccc ggcgtccagc
cctggcatcc cgcaccagca ccagcaccag caccaccggg cgctagcagg cgtcggcatg
gcggtcgggt gcgcggcgga ggcggccgtg gccgcggcgg gtgtcgcggg gacgaggtgc
ggggcgcacg acggggaggt gcctatggag gtggcgcggc accacgagca cgcggagcca
gggtcggggc ggtgctgctc cgcggtggtc cagcacgtag cggcgccggc ggcggcggtg
tggtcggtgg tgcggcggtt cgaccagccc caggcgtaca agcggttcgt ccgcagctgc
gcgctgctcg ccggggacgg cggcgtgggc acgctccgcg aggtgcgcgt cgtgtcgggc
ctccccgcgg cgtcctcccg cgagcgcctc gagatcctcg acgacgagag ccacgtcctc
agcttccgcg tcgtcggcgg cgagcaccgc ctcaagaact acctctcggt caccaccgtc
cacccgtccc cgtccgcgcc gacggccgcc accgtcgtgg tggagtccta cgtcgtcgac
gtgcccccgg gcaacacgcc cgaggacacc cgcgtgttcg tcgacaccat cgtcaagtgc
aacctccagt ctctcgccaa gaccgccgag aagctcgccg ccggcgcgag ggccgccggc
tcgtgagcgc tctccaccgc cgggccaccg atcgtttttt tccgggcgga tggatgtgtg
tttcttcggc gcgtgagagc tttctccttt ttttccccct ttttcctctt tggtttcttc
acatttgacc ccctcttttt tttcctcttt atttttttcc ctcgttgttg cgagagacac
acaaacacca tccacccttg tccaaaaaga ttcaagaaat gacatgtata attgacagcc
tgcaattcca tttgtatgct cgagttggtt ggtgtattga gtacatacat acatatacat
aaacacgata gtagtattag atatttagat ctgttctctg gcacaaattt aaagtgtacg
ttcgtattta aagttgtgct acatctatac agc
SEQ ID NO: 36: PYL4 Oryza sativa Japonica Group nucleic acid
sequence (cDNA; coding sequence)
atgccgtgca tcccggcgtc cagccctggc atcccgcacc agcaccagca ccagcaccac
cgggcgctag caggcgtcgg catggcggtc gggtgcgcgg cggaggcggc cgtggccgcg
gcgggtgtcg cggggacgag gtgcggggcg cacgacgggg aggtgcctat ggaggtggcg
cggcaccacg agcacgcgga gccagggtcg gggcggtgct gctccgcggt ggtccagcac
gtagcggcgc cggcggcggc ggtgtggtcg gtggtgcggc ggttcgacca gccccaggcg
tacaagcggt tcgtccgcag ctgcgcgctg ctcgccgggg acggcggcgt gggcacgctc
cgcgaggtgc gcgtcgtgtc gggcctcccc gcggcgtcct cccgcgagcg cctcgagatc
ctcgacgacg agagccacgt cctcagcttc cgcgtcgtcg gcggcgagca ccgcctcaag
aactacctct cggtcaccac cgtccacccg tccccgtccg cgccgacggc cgccaccgtc
gtggtggagt cctacgtcgt cgacgtgccc ccgggcaaca cgcccgagga cacccgcgtg
ttcgtcgaca ccatcgtcaa gtgcaacctc cagtctctcg ccaagaccgc cgagaagctc
SEQ ID NO: 37: PYL4 Oryza sativa Japonica Group protein sequence
mpcipasspg iphqhqhqhh ralagvgmav gcaaeaavaa agvagtrcga hdgevpmeva
rhhehaepgs grccsavvqh vaapaaavws vvrrfdqpqa ykrfvrscal lagdggvgtl
revrvvsglp aassrerlei lddeshvlsf rvvggehrlk nylsvttvhp spsaptaatv
vvesyvvdvp pgntpedtrv fvdtivkcnl qslaktaekl aagaraags
SEQ ID NO: 38: PYL4 Triticum aestivum nucleotide sequence
(genomic/cDNA; coding sequence); GenBank: GAEF01112574.1
cgcgagcg gctcgagatc ctggacgacg agagccacgt gctcagtttc cgcgtcgtcg
gtggcgagca ccggctcaag aactacctct ccgtcaccac cgtccacccg tccccggccg
cgccgtccag cgccaccgtc gtcgtggagt cgtacgtcgt ggacgtgccc gcgggcaaca
cgaccgagga cacccgcgtg ttcatcgaca ccatcgtcaa gtgcaacctc
SEQ ID NO: 39: PYL4 Triticum aestivum partial protein sequence
RERLEILDDE SHVLSFRVVG GEHRLKNYLS VTTVHPSPAA PSSATVVVES YVVDVPAGNT
TEDTRVFIDT IVKCNL
SEQ ID NO: 40: PYL4 Citrus sinensis nucleotide sequence (genomic;
coding sequence including the 5′ and 3′ UTR)
cgtcgcttcg acaaccccca ggcttacaag cacttcgtca agagctgcca cgtcattaac
ggcgacggcg acgtgggcac cctccgggag gtccacgtca tctccggact ccccgccggc
cgcagcacgg agaggctcga gatcctagac gacgagcgcc acgtcataag cttcagcgtc
atcgggggcg accacaggct ggcgaattac aggtcggtga cgactctcca cccatcgccc
gctggcaacg ggacggtggt cgtggagtct tacgtcgtcg acgtgcctcc cggaaacacg
aacgaagaca cctgcgtgtt cgtcgacacc atcgttaaat gcaacctcca gtcgctggcg
cagaccgcgg agaatctgac caggcgaaac aacaacaacc acagcagtaa caacagcagc
atcagcagca acaacaacgg cccgatcagg tcatgttctg ttctcgagtg aatagttcta
gttactagta ttacaaaata gtgttgatgt ctttggtaat acgcagtcgt ttttgtgagt
atccgatgtg gattgcctaa agaaactaca gaatcatctc gtttctgtta actacttctt
catcatgttc atttggttac tgcatttttc ttttttcttt tttttttaaa ttattaatgt
gaaatatttt taattgataa ataataatgt cgacctacag c
SEQ ID NO: 41: PYL4 Citrus sinensis nucleotide sequence
(cDNA; coding sequence)
atgccagtaa atccaccgaa atcatctcta ttgctgcaca gaatcaacaa cgtaaacaca
gccaccaaca cgatcgcaac agcaacagcc aacatgcttt gccagaaaga gcagcttcag
ttccaaaagc gcttcccagc aacgtggtca acccccgtcc ccgacgccgt ggcacgccac
cacaccctcg tcgttggccc caaccagtgc tgctcctccg tcgtccagca gatcgccgct
cccgtctcga ccgtctggtc cgtcgtccgt cgcttcgaca acccccaggc ttacaagcac
ttcgtcaaga gctgccacgt cattaacggc gacggcgacg tgggcaccct ccgggaggtc
cacgtcatct ccggactccc cgccggccgc agcacggaga ggctcgagat cctagacgac
gagcgccacg tcataagctt cagcgtcatc gggggcgacc acaggctggc gaattacagg
tcggtgacga ctctccaccc atcgcccgct ggcaacggga cggtggtcgt ggagtcttac
gtcgtcgacg tgcctcccgg aaacacgaac gaagacacct gcgtgttcgt cgacaccatc
gttaaatgca acctccagtc gctggcgcag accgcggaga atctgaccag gcgaaacaac
aacaaccaca gcagtaacaa cagcagcatc agcagcaaca acaacggccc gatcaggtca
SEQ ID NO: 42: PYL4 Citrus sinensis protein sequence
MPVNPPKSSL LLHRINNVNT ATNTIATATA NMLCQKEQLQ FQKRFPATWS TPVPDAVARH
HTLVVGPNQC CSSVVQQIAA PVSTVWSVVR RFDNPQAYKH FVKSCHVING DGDVGTLREV
HVISGLPAGR STERLEILDD ERHVISFSVI GGDHRLANYR SVTTLHPSPA GNGTVVVESY
VVDVPPGNTN EDTCVFVDTI VKCNLQSLAQ TAENLTRRNN NNHSSNNSSI SSNNNGPIRS
CSVLE
SEQ ID NO: s: 43-54 are At protein sequences
SEQ ID NO: 43: PYL1 amino acid sequence, NP_199491.2; At5g46790
Met Ala Asn Ser Glu Ser Ser Ser Ser Pro Val Asn Glu Glu Glu Asn
Ser Gln Arg Ile Ser Thr Leu His His Gln Thr Met Pro Ser Asp Leu
Thr Gln Asp Glu Phe Thr Gln Leu Ser Gln Ser Ile Ala Glu Phe His
Thr Tyr Gln Leu Gly Asn Gly Arg Cys Ser Ser Leu Leu Ala Gln Arg
Ile His Ala Pro Pro Glu Thr Val Trp Ser Val Val Arg Arg Phe Asp
Arg Pro Gln Ile Tyr Lys His Phe Ile Lys Ser Cys Asn Val Ser Glu
Asp Phe Glu Met Arg Val Gly Cys Thr Arg Asp Val Asn Val Ile Ser
Gly Leu Pro Ala Asn Thr Ser Arg Glu Arg Leu Asp Leu Leu Asp Asp
Asp Arg Arg Val Thr Gly Phe Ser Ile Thr Gly Gly Glu His Arg Leu
Arg Asn Tyr Lys Ser Val Thr Thr Val His Arg Phe Glu Lys Glu Glu
Glu Glu Glu Arg Ile Trp Thr Val Val Leu Glu Ser Tyr Val Val Asp
Val Pro Glu Gly Asn Ser Glu Glu Asp Thr Arg Leu Phe Ala Asp Thr
Val Ile Arg Leu Asn Leu Gln Lys Leu Ala Ser Ile Thr Glu Ala Met
Asn Arg Asn Asn Asn Asn Asn Asn Ser Ser Gln Val Arg
SEQ ID NO: 44: PYL2 polypeptide sequence; O80992
version 1; At2g26040
Met Ser Ser Ser Pro Ala Val Lys Gly Leu Thr Asp Glu Glu Gln Lys
Thr Leu Glu Pro Val Ile Lys Thr Tyr His Gln Phe Glu Pro Asp Pro
Thr Thr Cys Thr Ser Leu Ile Thr Gln Arg Ile His Ala Pro Ala Ser
Val Val Trp Pro Leu Ile Arg Arg Phe Asp Asn Pro Glu Arg Tyr Lys
His Phe Val Lys Arg Cys Arg Leu Ile Ser Gly Asp Gly Asp Val Gly
Ser Val Arg Glu Val Thr Val Ile Ser Gly Leu Pro Ala Ser Thr Ser
Thr Glu Arg Leu Glu Phe Val Asp Asp Asp His Arg Val Leu Ser Phe
Arg Val Val Gly Gly Glu His Arg Leu Lys Asn Tyr Lys Ser Val Thr
Ser Val Asn Glu Phe Leu Asn Gln Asp Ser Gly Lys Val Tyr Thr Val
Val Leu Glu Ser Tyr Thr Val Asp Ile Pro Glu Gly Asn Thr Glu Glu
Asp Thr Lys Met Phe Val Asp Thr Val Val Lys Leu Asn Leu Gln Lys
Leu Gly Val Ala Ala Thr Ser Ala Pro Met His Asp Asp Glu
SEQ ID NO: 45: PYL3 amino acid sequence; Q9SSM7
version 1; At1g73000
Met Asn Leu Ala Pro Ile His Asp Pro Ser Ser Ser Ser Thr Thr Thr
Thr Ser Ser Ser Thr Pro Tyr Gly Leu Thr Lys Asp Glu Phe Ser Thr
Leu Asp Ser Ile Ile Arg Thr His His Thr Phe Pro Arg Ser Pro Asn
Thr Cys Thr Ser Leu Ile Ala His Arg Val Asp Ala Pro Ala His Ala
Ile Trp Arg Phe Val Arg Asp Phe Ala Asn Pro Asn Lys Tyr Lys His
Phe Ile Lys Ser Cys Thr Ile Arg Val Asn Gly Asn Gly Ile Lys Glu
Ile Lys Val Gly Thr Ile Arg Glu Val Ser Val Val Ser Gly Leu Pro
Ala Ser Thr Ser Val Glu Ile Leu Glu Val Leu Asp Glu Glu Lys Arg
Ile Leu Ser Phe Arg Val Leu Gly Gly Glu His Arg Leu Asn Asn Tyr
Arg Ser Val Thr Ser Val Asn Glu Phe Val Val Leu Glu Lys Asp Lys
Lys Lys Arg Val Tyr Ser Val Val Leu Glu Ser Tyr Ile Val Asp Ile
Pro Gln Gly Asn Thr Glu Glu Asp Thr Arg Met Phe Val Asp Thr Val
Val Lys Ser Asn Leu Gln Asn Leu Ala Val Ile Ser Thr Ala Ser Pro
Thr
SEQ ID NO: 46: PYL5 amino acid sequence, At5g05440
Met Arg Ser Pro Val Gln Leu Gln His Gly Ser Asp Ala Thr Asn Gly
Phe His Thr Leu Gln Pro His Asp Gln Thr Asp Gly Pro Ile Lys Arg
Val Cys Leu Thr Arg Gly Met His Val Pro Glu His Val Ala Met His
His Thr His Asp Val Gly Pro Asp Gln Cys Cys Ser Ser Val Val Gln
Met Ile His Ala Pro Pro Glu Ser Val Trp Ala Leu Val Arg Arg Phe
Asp Asn Pro Lys Val Tyr Lys Asn Phe Ile Arg Gln Cys Arg Ile Val
Gln Gly Asp Gly Leu His Val Gly Asp Leu Arg Glu Val Met Val Val
Ser Gly Leu Pro Ala Val Ser Ser Thr Glu Arg Leu Glu Ile Leu Asp
Glu Glu Arg His Val Ile Ser Phe Ser Val Val Gly Gly Asp His Arg
Leu Lys Asn Tyr Arg Ser Val Thr Thr Leu His Ala Ser Asp Asp Glu
Gly Thr Val Val Val Glu Ser Tyr Ile Val Asp Val Pro Pro Gly Asn
Thr Glu Glu Glu Thr Leu Ser Phe Val Asp Thr Ile Val Arg Cys Asn
Leu Gln Ser Leu Ala Arg Ser Thr Asn Arg Gln
SEQ ID NO: 47: PYL6 amino acid sequence. At2g40330
Met Pro Thr Ser Ile Gln Phe Gln Arg Ser Ser Thr Ala Ala Glu Ala
Ala Asn Ala Thr Val Arg Asn Tyr Pro His His His Gln Lys Gln Val
Gln Lys Val Ser Leu Thr Arg Gly Met Ala Asp Val Pro Glu His Val
Glu Leu Ser His Thr His Val Val Gly Pro Ser Gln Cys Phe Ser Val
Val Val Gln Asp Val Glu Ala Pro Val Ser Thr Val Trp Ser Ile Leu
Ser Arg Phe Glu His Pro Gln Ala Tyr Lys His Phe Val Lys Ser Cys
His Val Val Ile Gly Asp Gly Arg Glu Val Gly Ser Val Arg Glu Val
Arg Val Val Ser Gly Leu Pro Ala Ala Phe Ser Leu Glu Arg Leu Glu
Ile Met Asp Asp Asp Arg His Val Ile Ser Phe Ser Val Val Gly Gly
Asp His Arg Leu Met Asn Tyr Lys Ser Val Thr Thr Val His Glu Ser
Glu Glu Asp Ser Asp Gly Lys Lys Arg Thr Arg Val Val Glu Ser Tyr
Val Val Asp Val Pro Ala Gly Asn Asp Lys Glu Glu Thr Cys Ser Phe
Ala Asp Thr Ile Val Arg Cys Asn Leu Gln Ser Leu Ala Lys Leu Ala
Glu Asn Thr Ser Lys Phe Ser
SEQ ID NO: 48: PYL7 amino acid sequence; At4g01026
Met Glu Met Ile Gly Gly Asp Asp Thr Asp Thr Glu Met Tyr Gly Ala
Leu Val Thr Ala Gln Ser Leu Arg Leu Arg His Leu His His Cys Arg
Glu Asn Gln Cys Thr Ser Val Leu Val Lys Tyr Ile Gln Ala Pro Val
His Leu Val Trp Ser Leu Val Arg Arg Phe Asp Gln Pro Gln Lys Tyr
Lys Pro Phe Ile Ser Arg Cys Thr Val Asn Gly Asp Pro Glu Ile Gly
Cys Leu Arg Glu Val Asn Val Lys Ser Gly Leu Pro Ala Thr Thr Ser
Thr Glu Arg Leu Glu Gln Leu Asp Asp Glu Glu His Ile Leu Gly Ile
Asn Ile Ile Gly Gly Asp His Arg Leu Lys Asn Tyr Ser Ser Ile Leu
Thr Val His Pro Glu Met Ile Asp Gly Arg Ser Gly Thr Met Val Met
Glu Ser Phe Val Val Asp Val Pro Gln Gly Asn Thr Lys Asp Asp Thr
Cys Tyr Phe Val Glu Ser Leu Ile Lys Cys Asn Leu Lys Ser Leu Ala
Cys Val Ser Glu Arg Leu Ala Ala Gln Asp Ile Thr Asn Ser Ile Ala
Thr Phe Cys Asn Ala Ser Asn Gly Tyr Arg Glu Lys Asn His Thr Glu
Thr Asn Leu
SEQ ID NO: 49: PYL8 amino acid sequence; At5g53160
Met Glu Ala Asn Gly Ile Glu Asn Leu Thr Asn Pro Asn Gln Glu Arg
Glu Phe Ile Arg Arg His His Lys His Glu Leu Val Asp Asn Gln Cys
Ser Ser Thr Leu Val Lys His Ile Asn Ala Pro Val His Ile Val Trp
Ser Leu Val Arg Arg Phe Asp Gln Pro Gln Lys Tyr Lys Pro Phe Ile
Ser Arg Cys Val Val Lys Gly Asn Met Glu Ile Gly Thr Val Arg Glu
Val Asp Val Lys Ser Gly Leu Pro Ala Thr Arg Ser Thr Glu Arg Leu
Glu Leu Leu Asp Asp Asn Glu His Ile Leu Ser Ile Arg Ile Val Gly
Gly Asp His Arg Leu Lys Asn Tyr Ser Ser Ile Ile Ser Leu His Pro
Glu Thr Ile Glu Gly Arg Ile Gly Thr Leu Val Ile Glu Ser Phe Val
Val Asp Val Pro Glu Gly Asn Thr Lys Asp Glu Thr Cys Tyr Phe Val
Glu Ala Leu Ile Lys Cys Asn Leu Lys Ser Leu Ala Asp Ile Ser Glu
Arg Leu Ala Val Gln Asp Thr Thr Glu Ser Arg Val
SEQ ID NO: 50: PYL9 amino acid sequence; At1g01360
Met Met Asp Gly Val Glu Gly Gly Thr Ala Met Tyr Gly Gly Leu Glu
Thr Val Gln Tyr Val Arg Thr His His Gln His Leu Cys Arg Glu Asn
Gln Cys Thr Ser Ala Leu Val Lys His Ile Lys Ala Pro Leu His Leu
Val Trp Ser Leu Val Arg Arg Phe Asp Gln Pro Gln Lys Tyr Lys Pro
Phe Val Ser Arg Cys Thr Val Ile Gly Asp Pro Glu Ile Gly Ser Leu
Arg Glu Val Asn Val Lys Ser Gly Leu Pro Ala Thr Thr Ser Thr Glu
Arg Leu Glu Leu Leu Asp Asp Glu Glu His Ile Leu Gly Ile Lys Ile
Ile Gly Gly Asp His Arg Leu Lys Asn Tyr Ser Ser Ile Leu Thr Val
His Pro Glu Ile Ile Glu Gly Arg Ala Gly Thr Met Val Ile Glu Ser
Phe Val Val Asp Val Pro Gln Gly Asn Thr Lys Asp Glu Thr Cys Tyr
Phe Val Glu Ala Leu Ile Arg Cys Asn Leu Lys Ser Leu Ala Asp Val
Ser Glu Arg Leu Ala Ser Gln Asp Ile Thr Gln
SEQ ID NO: 51: PYL10 amino acid sequence; At4g27920
Met Asn Gly Asp Glu Thr Lys Lys Val Glu Ser Glu Tyr Ile Lys Lys
His His Arg His Glu Leu Val Glu Ser Gln Cys Ser Ser Thr Leu Val
Lys His Ile Lys Ala Pro Leu His Leu Val Trp Ser Ile Val Arg Arg
Phe Asp Glu Pro Gln Lys Tyr Lys Pro Phe Ile Ser Arg Cys Val Val
Gln Gly Lys Lys Leu Glu Val Gly Ser Val Arg Glu Val Asp Leu Lys
Ser Gly Leu Pro Ala Thr Lys Ser Thr Glu Val Leu Glu Ile Leu Asp
Asp Asn Glu His Ile Leu Gly Ile Arg Ile Val Gly Gly Asp His Arg
Leu Lys Asn Tyr Ser Ser Thr Ile Ser Leu His Ser Glu Thr Ile Asp
Gly Lys Thr Gly Thr Leu Ala Ile Glu Ser Phe Val Val Asp Val Pro
Glu Gly Asn Thr Lys Glu Glu Thr Cys Phe Phe Val Glu Ala Leu Ile
Gln Cys Asn Leu Asn Ser Leu Ala Asp Val Thr Glu Arg Leu Gln Ala
Glu Ser Met Glu Lys Lys Ile
SEQ ID NO: 52: PYL11 amino acid sequence; At5g45860
Met Glu Thr Ser Gln Lys Tyr His Thr Cys Gly Ser Thr Leu Val Gln
Thr Ile Asp Ala Pro Leu Ser Leu Val Trp Ser Ile Leu Arg Arg Phe
Asp Asn Pro Gln Ala Tyr Lys Gln Phe Val Lys Thr Cys Asn Leu Ser
Ser Gly Asp Gly Gly Glu Gly Ser Val Arg Glu Val Thr Val Val Ser
Gly Leu Pro Ala Glu Phe Ser Arg Glu Arg Leu Asp Glu Leu Asp Asp
Glu Ser His Val Met Met Ile Ser Ile Ile Gly Gly Asp His Arg Leu
Val Asn Tyr Arg Ser Lys Thr Met Ala Phe Val Ala Ala Asp Thr Glu
Glu Lys Thr Val Val Val Glu Ser Tyr Val Val Asp Val Pro Glu Gly
Asn Ser Glu Glu Glu Thr Thr Ser Phe Ala Asp Thr Ile Val Gly Phe
Asn Leu Lys Ser Leu Ala Lys Leu Ser Glu Arg Val Ala His Leu Lys
Leu
SEQ ID NO: 53: PYL12 amino acid sequence; At5g45870
Met Lys Thr Ser Gln Glu Gln His Val Cys Gly Ser Thr Val Val Gln
Thr Ile Asn Ala Pro Leu Pro Leu Val Trp Ser Ile Leu Arg Arg Phe
Asp Asn Pro Lys Thr Phe Lys His Phe Val Lys Thr Cys Lys Leu Arg
Ser Gly Asp Gly Gly Glu Gly Ser Val Arg Glu Val Thr Val Val Ser
Asp Leu Pro Ala Ser Phe Ser Leu Glu Arg Leu Asp Glu Leu Asp Asp
Glu Ser His Val Met Val Ile Ser Ile Ile Gly Gly Asp His Arg Leu
Val Asn Tyr Gln Ser Lys Thr Thr Val Phe Val Ala Ala Glu Glu Glu
Lys Thr Val Val Val Glu Ser Tyr Val Val Asp Val Pro Glu Gly Asn
Thr Glu Glu Glu Thr Thr Leu Phe Ala Asp Thr Ile Val Gly Cys Asn
Leu Arg Ser Leu Ala Lys Leu Ser Glu Lys Met Met Glu Leu Thr
SEQ ID NO: 54: PYL13 amino acid sequence; At4g18620
Met Glu Ser Ser Lys Gln Lys Arg Cys Arg Ser Ser Val Val Glu Thr
Ile Glu Ala Pro Leu Pro Leu Val Trp Ser Ile Leu Arg Ser Phe Asp
Lys Pro Gln Ala Tyr Gln Arg Phe Val Lys Ser Cys Thr Met Arg Ser
Gly Gly Gly Gly Gly Lys Gly Gly Glu Gly Lys Gly Ser Val Arg Asp
Val Thr Leu Val Ser Gly Phe Pro Ala Asp Phe Ser Thr Glu Arg Leu
Glu Glu Leu Asp Asp Glu Ser His Val Met Val Val Ser Ile Ile Gly
Gly Asn His Arg Leu Val Asn Tyr Lys Ser Lys Thr Lys Val Val Ala
Ser Pro Glu Asp Met Ala Lys Lys Thr Val Val Val Glu Ser Tyr Val
Val Asp Val Pro Glu Gly Thr Ser Glu Glu Asp Thr Ile Phe Phe Val
Lys Met Met Lys
Nucleic acid sequence Arabidopsis PYL4^{A194T}, mutated codon in
bold and underlined. The second codon was modified to GTT to
get an NcoI site
SEQ ID NO: 55
ATGGTTGCCGTTCACCGTCCTTCTTCCGCCGTATCAGACGGAGATTCCGTTCAGA
TTCCGATGATGATCGCGTCGTTTCAAAAACGTTTTCCTTCTCTCTCACGCGACTC
CACGGCCGCTCGTTTTCACACACACGAGGTTGGTCCTAATCAGTGTTGCTCCGCC
GTTATTCAAGAGATCTCCGCTCCAATCTCCACCGTTTGGTCCGTCGTACGCCGCT
TTGATAACCCACAAGCTTACAAACACTTTCTCAAAAGCTGTAGCGTCATCGGCGG
AGACGGCGATAACGTTGGTAGCCTCCGTCAAGTCCACGTCGTCTCTGGTCTCCCC
GCCGCTAGCTCCACCGAGAGACTCGATATCCTCGACGACGAACGCCACGTCATCA
GCTTCAGCGTTGTTGGTGGTGACCACCGGCTCTCTAACTACCGATCCGTAACGAC
CCTTCACCCTTCTCCGATCTCCGGGACCGTCGTTGTCGAGTCTTACGTCGTTGAT
GTTCCTCCAGGCAACACAAAGGAAGAGACTTGTGACTTCGTTGACGTTATCGTAC
GATGCAATCTTCAATCTCTTGCGAAAATAACTGAGAATACTGCGGCTGAGAGCAA
GAAGAAGATGTCTCTGTGA
Nucleic acid sequence Arabidopsis PYL4^{H82R V97A}, mutated
codons in bold and underlined. The second codon was modified
to GTT to get an NcoI site
SEQ ID NO: 56
ATGGTTGCCGTTCACCGTCCTTCTTCCGCCGTATCAGACGGAGATTCCGTTCAGA
TTCCGATGATGATCGCGTCGTTTCAAAAACGTTTTCCTTCTCTCTCACGCGACTC
CACGGCCGCTCGTTTTCACACACACGAGGTTGGTCCTAATCAGTGTTGCTCCGCC
GTTATTCAAGAGATCTCCGCTCCAATCTCCACCGTTTGGTCCGTCGTACGCCGCT
TTGATAACCCACAAGCTTACAAACGCTTTCTCAAAAGCTGTAGCGTCATCGGCGG
AGACGGCGATAACGCTGGTAGCCTCCGTCAAGTCCACGTCGTCTCTGGTCTCCCC
GCCGCTAGCTCCACCGAGAGACTCGATATCCTCGACGACGAACGCCACGTCATCA
GCTTCAGCGTTGTTGGTGGTGACCACCGGCTCTCTAACTACCGATCCGTAACGAC
CCTTCACCCTTCTCCGATCTCCGGGACCGTCGTTGTCGAGTCTTACGTCGTTGAT
GTTCCTCCAGGCAACACAAAGGAAGAGACTTGTGACTTCGTTGACGTTATCGTAC
GATGCAATCTTCAATCTCTTGCGAAAATAGCCGAGAATACTGCGGCTGAGAGCAA
GAAGAAGATGTCTCTGTGA
AtPYL A194T- mutated residue in bold and underlined
SEQ ID NO. 60
MLAVHRPSSAVSDGDSVQIPMMIASFQKRFPSLSRDSTAARFHTHEVGPNQCCSAVIQEISAPISTVWSV
VRRFDNPQAYKHFLKSCSVIGGDGDNVGSLRQVHVVSGLPAASSTERLDILDDERHVISFSVVGGDHRLS
NYRSVTTLHPSPISGTVVVESYVVDVPPGNTKEETCDFVDVIVRCNLQSLAKITENTAAESKKKMSL
AtPYL V97A- mutated residue in bold and underlined
SEQ ID NO. 61
MLAVHRPSSAVSDGDSVQIPMMIASFQKRFPSLSRDSTAARFHTHEVGPNQCCSAVIQEISAPISTVWSV
VRRFDNPQAYKHFLKSCSVIGGDGDNAGSLRQVHVVSGLPAASSTERLDILDDERHVISFSVVGGDHRLS
NYRSVTTLHPSPISGTVVVESYVVDVPPGNTKEETCDFVDVIVRCNLQSLAKIAENTAAESKKKMSL
AtPYL H82 and V97A- mutated residue in bold and underlined
SEQ ID NO. 62
MLAVHRPSSAVSDGDSVQIPMMIASFQKRFPSLSRDSTAARFHTHEVGPNQCCSAVIQEISAPISTVWSV
VRRFDNPQAYKRFLKSCSVIGGDGDNAGSLRQVHVVSGLPAASSTERLDILDDERHVISFSVVGGDHRLS
NYRSVTTLHPSPISGTVVVESYVVDVPPGNTKEETCDFVDVIVRCNLQSLAKIAENTAAESKKKMSL
AtPYL F130Y and C176R- mutated residue in bold and underlined
SEQ ID NO. 63
MLAVHRPSSAVSDGDSVQIPMMIASFQKRFPSLSRDSTAARFHTHEVGPNQCCSAVIQEISAPISTVWSV
VRRFDNPQAYKHFLKSCSVIGGDGDNVGSLRQVHVVSGLPAASSTERLDILDDERHVISYSVVGGDHRLS
NYRSVTTLHPSPISGTVVVESYVVDVPPGNTKEETRDFVDVIVRCNLQSLAKIAENTAAESKKKMSL
AtPYL F130Y mutated residue in bold and underlined
SEQ ID NO. 64
MLAVHRPSSAVSDGDSVQIPMMIASFQKRFPSLSRDSTAARFHTHEVGPNQCCSAVIQEISAPISTVWSV
VRRFDNPQAYKHFLKSCSVIGGDGDNVGSLRQVHVVSGLPAASSTERLDILDDERHVISYSVVGGDHRLS
NYRSVTTLHPSPISGTVVVESYVVDVPPGNTKEETCDFVDVIVRCNLQSLAKIAENTAAESKKKMSL
AtPYL C176R- mutated residue in bold and underlined
SEQ ID NO. 65
MLAVHRPSSAVSDGDSVQIPMMIASFQKRFPSLSRDSTAARFHTHEVGPNQCCSAVIQEISAPISTVWSV
VRRFDNPQAYKHFLKSCSVIGGDGDNVGSLRQVHVVSGLPAASSTERLDILDDERHVISFSVVGGDHRLS
NYRSVTTLHPSPISGTVVVESYVVDVPPGNTKEETRDFVDVIVRCNLQSLAKIAENTAAESKKKMSL

1. An isolated mutant nucleic acid encoding a mutant plant PYL or PYR polypeptide comprising an amino acid substitution corresponding to
c) one or more of position A194, V97, C176 and/or F130 in PYL4 as set forth in SEQ ID NO:3 or
d) positions H82 and V97 in PYL4 as set forth in SEQ ID NO:3.

2. An isolated nucleic acid according to claim 1 wherein the mutant plant PYL or PYR polypeptide is a monocot or dicot PYL or PYR polypeptide.

3. An isolated nucleic acid according to claim 2 wherein said monocot or dicot plant is selected from maize, rice, wheat, oilseed rape, sorghum, soybean, potato, tomato, grape, barley, pea, bean, field bean, lettuce, cotton, sugar cane, sugar beet, broccoli or other vegetable brassicas or poplar.

4. An isolated nucleic acid according to claim 1 or 2 wherein the mutant PYL or PYR polypeptide is encoded by a nucleic acid comprising sequence SEQ ID NO:1, 2 or 4 a functional variant, ortholog or homolog thereof, but which has changes in one more codon to encode the substitutions as set forth in claim 1.

5. An isolated nucleic acid according to claim 4 wherein the mutant PYL or PYR polypeptide comprises SEQ ID NO:43 to 54.

6. An isolated nucleic acid according to claim 4 which encodes a polypeptide substantially as shown in SEQ ID NOs: 7, 9, 12, 14, 16, 19, 21, 23, 26, 28, 31, 34, 37, 39 or 42 but which has changes in one more codon to encode the substitutions as set forth in claim 1.

7. An isolated nucleic acid according to a preceding claim wherein the polypeptide comprises an amino acid substitution at position A194.

8. An isolated nucleic acid according to claim 7 wherein the polypeptide does not have further mutations that activate the PYL/PYR receptor in the absence of ABA.

9. An isolated nucleic acid according to claim 7 or claim 8 wherein the substitution is A194T.

10. An isolated nucleic acid according to any of claims 1-6 wherein the polypeptide comprises one or more amino acid substitutions at positions F130 and/or C176.

11. An isolated nucleic acid according to any of claims 1-6 wherein the substitution is V97.

12. An isolated nucleic acid according to any of claims 1-6 wherein the polypeptide comprises an amino acid substitution at position H82 and also comprises an amino acid substitution at position V97.

13. An isolated nucleic acid according to claim 12 wherein the substitutions are H82R and V97A.

14. An isolated nucleic acid according to claim 12 or 13 wherein the polypeptide does not have further mutations that activate the PYL/PYR receptor in the absence of ABA.

15. A vector comprising an isolated nucleic acid according to any of claims 1 to 14.

16. A vector according to claim 15 further comprising a regulatory sequence which directs expression of the nucleic acid.

17. A vector according to claim 15 wherein said regulatory sequence is a constitutive promoter, a strong promoter, an inducible promoter, a stress inducible promoter or a tissue specific promoter.

18. A vector according to claim 17 wherein said regulatory sequence is the CaMV35S promoter.

19. A vector according to claim 17 wherein said regulatory sequence is a stress inducible promoter.

20. A vector according to claim 19 wherein said stress inducible promoter is selected from Hahb1, RD29A or rabl7, P5CS1 or ABA- and drought-inducible promoters of Arabidopsis clade A PP2Cs, for example ABI1, ABI2, HAB1, PP2CA, HAI1, HAI2 and HAI3 or their corresponding crop orthologs.

21. A host cell comprising a vector according to any of claims 15 to 16.

22. A host cell according to claim 21 wherein said host cell is a bacterial or a plant cell.

23. A transgenic plant expressing an isolated nucleic acid according to any of claims 1 to 14 or comprising a vector according to any of claims 15 to 20.

24. A plant according to claim 21 wherein said plant is a crop plant or biofuel plant.

25. A plant according to claim 21 wherein said crop plant is selected from maize, rice, wheat, oilseed rape, sorghum, soybean, potato, tomato, grape, barley, pea, bean, field bean, lettuce, cotton, sugar cane, sugar beet, broccoli or other vegetable brassicas or poplar.

26. A product derived from a plant as defined in any of claims 23 to 25 or from a part thereof.

27. A method for increasing stress resistance in a transgenic plant comprising introducing and expressing a nucleic acid according to any of claims 1 to 14 or a vector according to any of claims 15 to 20 into a plant.

28. A method according to claim 27 wherein said stress is drought.

29. A method according to any of claims 257 to 28 wherein said stress is severe stress.

30. A method according to any of claims 27 to 28 wherein said stress is moderate stress.

31. A method for prolonging seed dormancy/preventing early germination/induce hyperdormancy in a transgenic plant comprising introducing and expressing a nucleic acid according to any of claims 1 to 14 or a vector according to any of claims 15 to 20 into a plant.

32. A method for constitutive activation of the ABA signalling pathway comprising introducing and expressing a nucleic acid according to any of claims 1 to 14 or a vector according to any of claims 15 to 20 into a plant.

33. A method for inhibiting the activity of a PP2C in a transgenic plant comprising introducing and expressing a nucleic acid according to any of claims 1 to 12 or a vector according to any of claims 15 to 20 into a plant.

34. A method according to claim 33 wherein said PP2CA is PP2CA.

35. A method for producing a transgenic plant with increased stress resistance comprising introducing and expressing a nucleic acid according to any of claims 1 to 14 or a vector according to any of claims 15 to 20 into a plant.

36. A plant obtained or obtainable by a method according to claim 34.

37. The use of a nucleic acid according to any of claims 1 to 14 or a vector according to any of claims 15 to 20 for increasing stress resistance, increasing water use efficiency, prolonging seed dormancy, increasing ABA-dependent inhibition of PP2C or activating the ABA signalling pathway in a plant.

38. A non-transgenic plant obtained by mutagenesis or genome editing comprising and expressing a PYL/PYR nucleic acid which encodes a PYL/PYR mutant polypeptide that has a different sequence compared to the wild type sequence. The mutant polypeptide comprises an amino acid substitutions corresponding to
a) one or more of position A194, V97, C176 and/or F130 in PYL4 as set forth in SEQ ID NO:3 or
b) positions H82 and V97 in PYL4 as set forth in SEQ ID NO:3.
